SEQUENCE LISTING (32 sequences)

SEQ ID NO: 1 - DNA, 2898 bp, Artificial Sequence
  Description of Artificial Sequence Synthetic pITI plasmid polylnucleotide
ccgcctaatgagcgggcttttttttcttaggccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaagccgccaccatggatgcaatgaagagagggctctgctgtgtgctgctgctgtgtggagcagtcttcgtttcgcccagcggtaccggatccgtcgacggggggagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 2 - DNA, 4157 bp, Cryptomeria japonica
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgaggatcagtcagcgcagatcatgctggatagcgtggtggagaagtacctgaggagtaacaggtcactgcgcaaggttgagcattccagacacgacgctatcaacatcttcaacgtggagaagtacggtgctgtcggagacgggaagcacgactgcaccgaagcct

SEQ ID NO: 3 - protein, 911 aa, Cryptomeria japonica
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLEDQSAQIMLDSVVEKYLRSNRSLRKVEHSRHDAINIFNVEKYGAVGDGKHDCTEAFSTAWQAACKNPSAMLLVPGSKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKDIKLSDISLKLTSGKIASCLNDNANGYFSGHVIPACKNLSPSAKRKESKSHKHPKTVMVENMRAYDKGNRTRILLGSRPPNCTNKCHGCSPCKAKLVIVHRIMPQEYYPQRWICSCHGKIYHPEFTLIPIAVGGALAGLVLIVLIAYLVGRKRSHAGYQTI

SEQ ID NO: 4 - DNA, 5326 bp, Cryptomeria japonica
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgaggacaatcctattgattcctgctggcgtggagattctaactgggcacagaaccggatgaaactggctgactgtgccgtgggctttggctcttccactatgggagggaagggaggcgacctgtacactgttacaaacagcgacgacgaccctgtcaatccagcacccggaaccttgagatatggtgcaacgcgagaccgaccactttggatcatctttagcggaaacatgaacatcaagttgaagatgcctatgtacatagctgggtacaaaaccttcgacggcagaggagcccaagtgtacattggcaacggaggtccctgcgtgttcatcaagcgtgttagtaatgtgatcattcacggtctgcacctctatggctgttcaacaagcgtgctggggaatgtgctgatcaatgagtcattcggtgttgaacccgtgcacccacaggacggtgatgcgttgacactgaggacagccaccaatatctggattgaccataacagtttctctaacagctcagatggcctggtggatgtcaccttgagtagcacaggggtcacaatcagcaacaatctgttcttcaaccatcataaggtgatgctgctgggccacgacgatgcgtattccgacgataagagcatgaaagtgacggtggcctttaaccagtttggtcctaactgtggacagcggatgcctagagccaggtacggactggtgcacgtggccaacaacaactatgatccgtggactatctatgcaattggcggttcttccaacccgacgatactgagtgaagggaactcctttaccgctcccaatgagagctacaagaagcaggtcaccatccgcataggctgcaaaactagttcatcctgtagcaactgggtgtggcagtccactcaagatgtcttctacaacggagcttacttcgttagcagtgggaaatacgaaggtggcaacatatacacaaagaaagaggctttcaatgtggagaatggcaatgccactccccagctcaccaagaatgcaggggtgctcacctgctccctgagcaaacggtgcgaattcacgctgatccccatcgctgtgggtggtgccctggcggggctggtcctcatcgtcctcatcgcctacctcgtcggcaggaagaggagtcacgcaggctaccagactatctagtaaagatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 5 - protein, 773 aa, Cryptomeria japonica
MAPRSARRPLLLLLLLLLLGLMHCASAAMFMVKNGNGTACIMANFSAAFSVNYDTKSGPKNMTLDLPSDATVVLNRSSCGKENTSDPSLVIAFGRGHTLTLNFTRNATRYSVQLMSFVYNLSDTHLFPNASSKEIKTVESITDIRADIDKKYRCVSGTQVHMNNVTVTLHDATIQAYLSNSSFSRGETRCEQDRPSPTTAPPAPPSPSPSPVPKSPSVDKYNVSGTNGTCLLASMGLQLNLTYERKDNTTVTRLLNINPNKTSASGSCGAHLVTLELHSEGTTVLLFQFGMNASSSRFFLQGIQLNTILPDARDPAFKAANGSLRALQATVGNSYKCNAEEHVRVTKAFSVNIFKVWVQAFKVEGGQFGSVEECLLDENSLEDNPIDSCWRGDSNWAQNRMKLADCAVGFGSSTMGGKGGDLYTVTNSDDDPVNPAPGTLRYGATRDRPLWIIFSGNMNIKLKMPMYIAGYKTFDGRGAQVYIGNGGPCVFIKRVSNVIIHGLHLYGCSTSVLGNVLINESFGVEPVHPQDGDALTLRTATNIWIDHNSFSNSSDGLVDVTLSSTGVTISNNLFFNHHKVMLLGHDDAYSDDKSMKVTVAFNQFGPNCGQRMPRARYGLVHVANNNYDPWTIYAIGGSSNPTILSEGNSFTAPNESYKKQVTIRIGCKTSSSCSNWVWQSTQDVFYNGAYFVSSGKYEGGNIYTKKEAFNVENGNATPQLTKNAGVLTCSLSKRCEFTLIPIAVGGALAGLVLIVLIAYLVGRKRSHAGYQTI

SEQ ID NO: 6 - DNA, 6817 bp, Artificial Sequence
  Description of Artificial Sequence Synthetic Cry J1&J2-LAMP polynucleotide
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgtccagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagccggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgaggacaatcctattgattcctgctggcgtggagattctaactgggcacagaaccggatgaaactggctgactgtgccgtgggctttggctcttccactatgggagggaagggaggcgacctgtacactgttacaaacagcgacgacgaccctgtcaatccagcacccggaaccttgagatatggtgcaacgcgagaccgaccactttggatcatctttagcggaaacatgaacatcaagttgaagatgcctatgtacatagctgggtacaaaaccttcgacggcagaggagcccaagtgtacattggcaacggaggtccctgcgtgttcatcaagcgtgttagtaatgtgatcattcacggtctgcacctctatggctgttcaacaagcgtgctggggaatgtgctgatcaatgagtcattcggtgttgaacccgtgcacccacaggacggtgatgcgttgacactgaggacagccaccaatatctggattgaccataacagtttctctaacagctcagatggcctggtggatgtcaccttgagtagcacaggggtcacaatcagcaacaatctgttcttcaaccatcataaggtgatgctgctgggccacgacgatgcgtattccgacgataagagcatgaaagtgacggtggcctttaaccagtttggtcctaactgtggacagcggatgcctagagccaggtacggactggtgcacgtggccaacaacaactatgatccgtggactatctatgcaattggcggttcttccaacccgacgatactgagtgaagggaactcctttaccgctcccaatgagagctacaagaagcaggtcaccatccgcataggctgcaaaactagttcatcctgtagcaactgggtgtggcagtccactcaagatgtcttctacaacggagcttacttcgttagcagtgggaaatacgaaggtggcaacatatacacaaagaaagaggctttcaatgtggagaatggcaatgccactccccagctcaccaagaatgcaggggtgctcacctgctccctgagcaaacggtgcggcggtggtggcctcgaggatcagtcagcgcagatcatgctggatagcgtggtggagaagtacctgaggagtaacaggtcactgcgcaaggttgagcattccagacacgacgctatcaacatcttcaacgtggagaagtacggtgctgtcggagacgggaagcacgactgcaccgaagccttttctacagcctggcaagctgcctgcaagaatccctcagccatgctcctcgtgcctgggtctaagaagtttgtcgtgaataaccttttcttcaatggaccctgccagccacactttaccttcaaagttgatgggatcatcgcagcctatcagaacccagctagctggaagaacaatcggatctggttgcagtttgccaaactgacaggattcaccctgatggggaaaggcgtgatcgacggacagggcaaacagtggtgggcagggcagtgcaagtgggtcaatggtagggagatttgcaatgacagggaccgtcctaccgctatcaagtttgatttcagcacaggactgattattcaggggttgaagctgatgaatagtccagagtttcaccttgtgtttggcaattgtgaaggtgtgaagatcataggcattagcattacagcacctcgcgattctcccaatacggacggcattgacatcttcgcctccaagaactttcacctgcaaaagaataccattggcacaggcgacgactgcgtggccattggcactggcagcagcaatatcgttatcgaagatttgatatgtggtcctgggcatggcataagcattggaagcctgggtagagaaaactcaagagctgaagtcagctatgttcacgttaacggagcgaagttcattgatacccagaacggactgcgaatcaaaacttggcaagggggaagtggcatggcatctcacatcatctacgagaacgtcgagatgatcaattccgagaaccccatactgattaaccaattctattgtacttccgcctctgcctgccagaatcagagatcagccgtgcagattcaggacgtgacatacaagaatatccgagggacgagcgctaccgctgccgcaatacagctcaaatgttccgatagcatgccctgcaaagatatcaagcttagtgatatctccctcaaactgactagcggaaagatagcgtcctgtctcaatgataacgcaaatggctacttctcagggcatgtgatccctgcatgcaaaaaccttagcccgagtgcgaaacgcaaagaatccaaatcccataagcatccgaagactgtgatggtcgagaacatgagagcctacgacaaagggaaccggacgaggattctgctgggctctcgaccgccaaactgtaccaacaaatgtcacggttgttctccatgcaaagctaaactggtgatagtgcatcgcatcatgcctcaagagtactatccccagcgttggatttgtagttgccatggcaagatctatcacccagaattcacgctgatccccatcgctgtgggtggtgccctggcggggctggtcctcatcgtcctcatcgcctacctcgtcggcaggaagaggagtcacgcaggctaccagactatctagtaaggatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 7 - protein, 1270 aa, Artificial Sequence [sequence omitted]

SEQ ID NO: 8 - DNA, 8023 bp, Artificial Sequence
  Description of Artificial Sequence Synthetic polynucleotide coding region for the Ara H1/H2/H3 polyprotein
ccgcctaatgagcgggcttttttttcttagggtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccccttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcctgcaaaccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggtaccccggctctagatggcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcgtcgacatggcgccccgcagcgcccggcgacccctgctgctgctactgctgttgctgctgctcggcctcatgcattgtgcgtcagcagcaatgtttatggtgaaaaatggcaacgggaccgcgtgcataatggccaacttctctgctgccttctcagtgaactacgacaccaagagtggccctaagaacatgacccttgacctgccatcagatgccacagtggtgctcaaccgcagctcctgtggaaaagagaacacttctgaccccagtctcgtgattgcttttggaagaggacatacactcactctcaatttcacgagaaatgcaacacgttacagcgttcagctcatgagttttgtttataacttgtcagacacacaccttttccccaatgcgagctccaaagaaatcaagactgtggaatctataactgacatcagggcagatatagataaaaaatacagatgtgttagtggcacccaggtccacatgaacaacgtgaccgtaacgctccatgatgccaccatccaggcgtacctttccaacagcagcttcagcaggggagagacacgctgtgaacaagacaggccttccccaaccacagcgccccctgcgccacccagcccctcgccctcacccgtgcccaagagcccctctgtggacaagtacaacgtgagcggcaccaacgggacctgcctgctggccagcatggggctgcagctgaacctcacctatgagaggaaggacaacacgacggtgacaaggcttctcaacatcaaccccaacaagacctcggccagcgggagctgcggcgcccacctggtgactctggagctgcacagcgagggcaccaccgtcctgctcttccagttcgggatgaatgcaagttctagccggtttttcctacaaggaatccagttgaatacaattcttcctgacgccagagaccctgcctttaaagctgccaacggctccctgcgagcgctgcaggccacagtcggcaattcctacaagtgcaacgcggaggagcacgtccgtgtcacgaaggcgttttcagtcaatatattcaaagtgtgggtccaggctttcaaggtggaaggtggccagtttggctctgtggaggagtgtctgctggacgagaacagcctcgagaagtccagcccctaccagaagaaaaccgagaacccctgcgcccagcggtgcctgcagtcttgtcagcaggaacccgacgacctgaagcagaaggcctgcgagagccggtgcaccaagctggaatacgaccccagatgcgtgtacgaccctagaggccacaccggcaccaccaaccagagaagccctccaggcgagcggaccagaggcagacagcctggcgactacgacgacgacagacggcagcccagaagagaagagggcggcagatggggacctgccggccctagagagagagaacgcgaggaagattggagacagcccagagaggactggcggaggccttctcaccagcagccccggaagatcagacccgagggcagagaaggcgagcaggaatggggcacacctggctctcacgtgcgcgaggaaaccagccggaacaaccccttctacttcccctcccggcggttcagcaccagatacggcaaccagaacggccggatcagagtgctgcagagattcgaccagcggagccggcagttccagaacctgcagaaccaccggatcgtgcagatcgaggccaagcccaacaccctggtgctgcccaaacacgccgacgccgacaacatcctcgtgatccagcagggccaggccaccgtgacagtggccaacggcaacaacagaaagagcttcaacctggacgagggccacgccctgagaatccccagcggcttcatcagctacatcctgaacagacacgacaatcagaacctgagggtggccaagatcagcatgcccgtgaacacccctggccagttcgaggacttcttccccgcatcctcccgggaccagagcagctacctgcagggcttcagccggaataccctggaagccgccttcaacgccgagttcaacgagatcagacgggtgctgctggaagagaacgctggcggagagcaggaagaacggggccagagaagatggtccaccagaagcagcgagaacaacgagggcgtgatcgtgaaggtgtccaaagaacacgtggaagaactgaccaagcacgccaagagcgtgtccaagaagggctccgaggaagagggggacatcaccaaccccatcaatctgagagagggcgagcccgacctgagcaacaacttcggcaagctgttcgaagtgaagcccgacaagaagaacccccagctgcaggacctggacatgatgctgacctgcgtggaaatcaaagagggggccctgatgctgccacacttcaactccaaagccatggtcatcgtggtcgtgaacaagggcaccggcaacctggaactggtggccgtgcggaaagagcagcagcagagaggccgcagagaggaagaagaggacgaggacgaagaagaagagggatccaaccgggaagtgcggcggtacaccgccagactgaaagaaggcgacgtgttcatcatgcctgccgcccaccccgtggccatcaatgcctctagcgagctgcatctgctgggcttcggcattaacgccgagaacaatcaccggatctttctggccggcgacaaagacaacgtgatcgaccagatcgagaagcaggccaaggacctggcctttcccggctctggcgaacaagtggaaaagctgatcaagaaccagaaagaaagccacttcgtgtccgccagaccccagagccagtctcagagccctagctcccccgagaaagagtctcctgagaaagaggaccaggaagaggaaaaccagggcggcaagggccctctgctgagcatcctgaaggccttcaatggcggcggaggcaggcagcagtgggaactgcagggcgacagaagatgccagtcccagctggaacgggccaacctgaggccttgcgagcagcacctgatgcagaaaatccagcgcgacgaggacagctacggccgggatccttacagccccagccaggacccttactcccctagccaggatcccgacagaagggacccctacagccctagcccctacgatagaagaggcgccggaagcagccagcaccaggaaagatgctgcaacgagctgaacgagtttgagaacaaccagcgctgcatgtgcgaggccctgcagcagatcatggaaaatcagagcgaccggctgcagggacggcagcaggaacagcagttcaagagagagctgcggaacctgccccagcagtgtggactgagagccccccagagatgcgacctggaagtggaaagcggcggcagagatcggtacggcggagggggcgtgaccttcagacagggcggagaagagaatgagtgccagtttcagcggctgaacgcccagaggcccgacaacagaatcgagagcgagggcggctacatcgagacatggaaccccaacaaccaggaatttcagtgcgctggggtggccctgagcaggaccgtgctgagaagaaatgccctgaggcggcccttctacagcaacgcccccctggaaatctacgtgcagcagggcagcggctacttcggcctgatctttcccggatgcccctccacctatgaggaacccgctcaggaaggcagacggtatcagagccagaagcctagcagacggttccaagtgggccaggacgatcccagccaacagcagcaggactctcaccagaaggtgcaccgcttcgacgagggcgacctgatcgctgtgccaaccggcgtggccttctggatgtacaacgacgaggataccgacgtcgtgaccgtgaccctgagcgacaccagctccatccacaaccagctggaccagttccccaggcggttttacctggccggcaatcaggaacaggaatttctgagataccagcagcagcagggctccagaccccactacagacagatcagccctagagtgcggggcgacgaacaggaaaatgagggcagcaacatcttctccggctttgcccaggaatttctgcagcacgccttccaggtggaccggcagaccgtggaaaacctgagaggcgagaacgagagagaggaacagggcgccatcgtgactgtgaagggcggcctgaggatcctgagccccgacgaagaggatgagtcctctagaagcccccccaaccgccgggaagagttcgatgaggaccgcagcagacctcagcagcgggggaagtacgacgagaacaggcggggctacaagaacggcatcgaggaaacaatctgcagcgccagcgtgaagaagaatctgggccggtccagcaaccccgacatctacaatccacaggccggcagcctgcggagcgtgaacgaactggatctgcccatcctgggatggctgggcctgtctgcccagcacggcaccatctaccggaacgccatgttcgtgcctcactacaccctgaatgcccacaccatcgtggtggctctgaacggccgcgcccacgtccaagtggtggacagcaacggcaatcgggtgtacgatgaagaactgcaggaaggacacgtcctggtggtgccccagaattttgccgtggccgccaaggcccagtccgagaactatgagtatctggccttcaagaccgacagccggccctctatcgccaatcaagccggcgagaacagcatcatcgacaacctgcccgaggaagtggtggccaacagctaccggctgcctagagagcaggcccggcagctgaagaacaacaaccctttcaagttcttcgtgcccccattcgaccaccagagcatgagagaggtggccgaattcacgctgatccccatcgctgtgggtggtgccctggcggggctggtcctcatcgtcctcatcgcctacctcgtcggcaggaagaggagtcacgcaggctaccagactatctagtaaggatctttttccctctgccaaaaattatggggacatcatgaagccccttgagcatctgacttctggctaataaaggaaatttattttcattgcaatagtgtgttggaattttttgtgtctctcactcggaaggacataagggcggccgctagc

SEQ ID NO: 9 - protein, 1672 aa, Artificial Sequence [sequence omitted]

SEQ ID NO: 10 - protein, 225 aa, Cryptomeria japonica
MAKVSDLALLLVAGMAISLYIQETGAVKFDIKNQCGYTVWAAGLPGGGQQLTQGQTWTVNLAAGTQSARFWGRTGCSFDASGKGTCQTGDCGGQLSCTVSGAVPATLAEYTQSDQDYYDVSLVDGFNIPLSINPTNAQCTAPACKADVNAVCPAELKVDGGCKSACAAFQTDQYCCTGTYANSCPATNYSMIFKNQCPQAYSYPKDDTATFACPSGTDYSIVFCP

SEQ ID NO: 11 - protein, 281 aa, Cryptomeria japonica
MGIMATQNSKSNIFWSSSASVVLVLLLLVDVGVCQNCGCNGLCCSQYGYCGSGEAYCGAGCKEGPCSSSSPPSTGTGVGSIVSSDVFNSIVGGAASGCAGNGFYTYDSFISAANAFNGFGTSGSSDVNKREIAAFFANAAHETGGFCYIEEQNPTSIYCDASNTQYPCASGKTYHGRGPLQLSWNYNYGAAGSYIQFDGLNNPEIVGTDSTISFKTAVWFWMVNSNCHTAITSGQGFGATIRAINSMECDGGNAATVASRVNYYQKFCQQLNVDTGSALQC

SEQ ID NO: 12 - protein, 306 aa, Cryptomeria japonica
MGGSRVLIIGGTGYIGRHVTNASLAQGHPTFLLVREITPSNPEKAQLLESFTSKGATLVQGSIDDHASLVAALKKVDVVISTLGAPQIADQFNLIKAIKEVGTIKRFFPSEFGNDVDKHHAVEPMKSMFDLKIKLRRTIEAEGIPHTYVVPHCFAGYFLTNLAQLGLAAPPRDKIVIYGDGTTKAVYMKEEDIGTFTIKAVDDPRTLNKTLYLKPPANTISTNDLVALWEAKIGKTLEKVYLSEEQVLKLLQDTPFPGTFMVSIFHTIYVKGDQTNFQIGPDGVEASALYPDVKYTTVEEYISAFV

SEQ ID NO: 13 - protein, 165 aa, Cryptomeria japonica
MAMRMKSSSMSSYRFSYCQMMLVLMVMTLVQIGAAQSDTNSCVNSLVPCASYLNATTKPPDSCCVPLLNVIQTQQQCLCNLLNSSIVKQSSINITQALNIPRLCGDTNVSTDACSTNATANAPSASTTPSVPADTGDSSGIGATSLQIFLPLLAVFFLGVFKSFP

SEQ ID NO: 14 - protein, 472 aa, Cryptomeria japonica
MARRLCSFLLSFLIIVSVWAENSKFARLNLASFTWKDAEDNKNCSAGELETSSLSVMHIQGKCSPFRLLNSSWWTAVSESIKGDTARYRAMVKGGWSAGKTMVNPQEDADIPLASGQAESSSNYIIKLGFGTPPQSFYTVLDTGSNIAWIPCNPCSGCSSKQQPFEPSKSSTYNYLTCASQQCQLLRVCTKSDNSVNCSLTQRYGDQSEVDEILSSETLSVGSQQVENFVFGCSNAARGLIQRTPSLVGFGRNPLSFVSQTATLYDSTFSYCLPSLFSSAFTGSLLLGKEALSAQGLKFTPLLSNSRYPSFYYVGLNGISVGEELVSIPAGTLSLDESTGRGTIIDSGTVITRLVEPAYNAMRDSFRSQLSNLTMASPTDLFDTCTNRPSGDVEFPLITLHFDDNLDLTLPLDNILYPGNDDGSVLCLAFGLPPGGGDDVLSTFGNYQQQKLRIVHDVAESRLGIASGNCDG

SEQ ID NO: 15 - protein, 348 aa, Cryptomeria japonica
MELLKQHRYMFLLISCIVILLNSMHADCEQIGVNYGMDGNNLPSAGDVVSLMKKNNIGKMRIFGPNADVLRAFANSRIEVIVGVENKGLEAVASSQDSANGWVNDNIKPFYPSTNIKYIAVGNEVLEMPDNAQYVSFLVPAIKNIQTALENANLQNNIKVSTAHAMTVIGTSSPPSKGTFKDAVKDSMSSILQFLQDHGSPFMANVYPYFSYDGDRSIKLDYALFNPTPPVVDEGLSYTNLFDAMVDAVLSAMESLGHPNIPIVITESGWPSAGKSVATIENAQTYNNNLIKHVLSNAGTPKRPGSSIETYIFALFNENLKGPAEVEKHFGLFNPDEQPVYPVKFSLN

SEQ ID NO: 16 - protein, 375 aa, Chamaecyparis obtusa
MASCTLLAVLVFLCAIVSCFSDNPIDSCWRGDANWDQNRMKLADCAVGFGSSAMGGKGGAFYTVTSSDDDPVNPAPGTLRYGATRERSLWIIFSKNLNIKLNMPLYIAGNKTIDGRGAEVHIGNGGPCLFMRTVSHVILHGLNIHGCNTSVSGNVLISEASGVVPVHAQDGDAITMRNVTDVWIDHNSLSDSSDGLVDVTLASTGVTISNNHFFNHHKVMLLGHSDIYSDDKSMKVTVAFNQFGPNAGQRMPRARYGLIHVANNNYDPWSIYAIGGSSNPTILSEGNSFTAPNDSDKKEVTRRVGCESPSTCANWVWRSTQDSFNNGAYFVSSGKNEGTNIYNNNEAFKVENGSAAPQLTKNAGVLTCILSKPCS

SEQ ID NO: 17 - protein, 367 aa, Juniperus ashei
MASPCLIAVLVFLCAIVSCYSDNPIDSCWRGDSNWDQNRMKLADCAVGFGSSTMGGKGGDFYTVTSTDDNPVNPTPGTLRYGATREKALWIIFSQNMNIKLKMPLYVAGHKTIDGRGADVHLGNGGPCLFMRKVSHVILHSLHIHGCNTSVLGDVLVSESIGVEPVHAQDGDAITMRNVTNAWIDHNSLSDCSDGLIDVTLGSTGITISNNHFFNHHKVMLLGHDDTYDDDKSMKVTVAFNQFGPNAGQRMPRARYGLVHVANNNYDPWNIYAIGGSSNPTILSEGNSFTAPSESYKKEVTKRIGCESPSACANWVWRSTRDAFINGAYFVSSGKTEETNIYNSNEAFKVENGNAAPQLTKNAGVVT

SEQ ID NO: 18 - protein, 367 aa, Juniperus virginiana
MASPCLIAFLVFLCAIVSCCSDNPIDSCWRGDSNWGQNRMKLADCAVGFGSSTMGGKGGDFYTVTSADDNPVNPTPGTLRYGATREKTLWIIFSQNMNIKLKMPLYVAGHKTIDGRGADVHLGNGGPCLFMRKVSHVILHGLHIHGCNTSVLGDVLVSESIGVVPVHAQDGDAITMRNVTNAWIDHNSLSDCSDGLIDVTLGSTGITIFNNHFFNHHKVMLLGHDDTYDDDKSMKVTVAFNQFGPNAGQRMPRARYGLVHVANNNYDPWNIYAIGGSSNPTILSEGNSFTAPNENYKYEVTKRIGCESTSACANWVWRSTRDAFSNGAYFVSSGKIEETNIYNSNEAFKVENGNAAPQLTKNAGVVA

SEQ ID NO: 19 - protein, 367 aa, Hexalectris arizonica
MASPCLVAVLVFLCAIVSCYSDNPIDSCWRGDSNWDQNRMKLADCVVGFGSLTMGGKGGEIYTVTSSDDNPVNPTPGTLRYGATREKALWIIFSQNMNIKLQMPLYVAGYKTIDGRGADVHLGNGGPCLFMRTASHVILHGLHIHGCNTSVLGDVLVSESIGVEPVHAQDGDAITMRNVTNAWIDHNSLSDCSDGLIDVTLGSTGITISNNHFFNHHKVMLLGHDDTYDDDISMKVTVAFNQFGPNAGQRMPRARYGLVHVANNNYDQWNIYAIGGSSNPTILSEGNSPTAPSESYKKEVTKRIGCESTSACANWVWRFTRDAFTNGAYFVSSGKAEETNIYNSNEAFKVENGNAAPQLTQNAGVVT

SEQ ID NO: 20 - protein, 366 aa, Juniperus oxycedrus
MASPCLRAVLVFLCAIVSCYSDNPIDSCWRGDSNWGQNRMKLADCVVGFGSSTMGGKGGEFYTVTSAEDNPVNPTPGTLRYGATREKALWIIFSQNMNIKLKMPLYVAGHKTIDGRGADVHLGNGGPCLFMRKVSHVILHGLHIGCNTSVLGDVLVSESIGVEPVHAQDGDAITMRNVTNAWIDHNSLSDCSDGLIDVTLGSTGITISNNHFFNHHKVMLLGHDDTYDNDKSMKVTVAFNQFGPNAGQRMPRARYGLVHVANNNYDPWNIYAIGGSSNPTILSEGNSFTAPSESYKKEVTKRIGCESTSACANWVWRSTRDAFTNGAYFVSSGKIEETNIYNSNEAFKVENGNAAPQLTKNAGVVT

SEQ ID NO: 21 - protein, 367 aa, Cupressus sempervirens
MDSPCLIAVLVFLCAIVSCYSDNPIDSCWRGDSNWDQNRMKLADCAVGFGSSTMGGKGGDIYTVTSAEDNPVNPTPGTLRYGATREKALWIIFSQNMNIKLKMPLYVAGHKTIDGRGADVHLGNGGPCLFMRKVSHVILHGLHIHGCNTSVLGNVLVSESIGVEPVHAQDGDAITMRNVTNAWIDHNSLSDCSDGLIDVTLSSTGITISNNHFFNHHKVMLLGHDDTYDDDKSMKVTVAFNQFGPNAGQRMPRARYGLVHVANNNYDQWNIYAIGGSSNPTILSEGNSFAAPNENYKKEVTKRIGCVSTSACANWVWRSTRDAFSNGAYFVSSGKTEETNIYTSNEAFKVENGNLAPQLTKNAGVVA

SEQ ID NO: 22 - protein, 514 aa, Chamaecyparis obtusa
MGMKFMAAVAFLALQLIVMAAAEDQSAQIMLDSDIEEYLRSNRSLKKLVHSRHDAATVFNVEQYGAVGDGKHDSTEAFATTWNAACKKASAVLLVPANKKFFVNNLVFRGPCQPHLSFKVDGTIVAQPDPARWKNSKIWLQFAQLTDFNLMGTFVIDGQGQQWWAGQCKVVNGRTVCNDRNRPTAIKIDYSKSVTVKELTLMNSPEFHLVFGECEGVKIQGLKIKAPRDSPNTDGIDIFASKRFHIEKCVIGTGDDCIAIGTGSSNITIKDLICGPGHGISIGSLGRDNSRAEVSHVHVNRAKFIDTQNGLRIKTWQGGSGLASYITYENVEMINSENPILINQFYCTSASACQNQRSAVQIQGVTYKNIHGTSATAAAIQLMCSDSVPCTGIQLSNVSLKLTSGKPASCVDKNARGFYSGRLIPTCKNLRPGPSPKEFELQQQPTTVMDENKGACAKGDSTCISLSSSPPNCKNKCKGCQPCKPKLIIVHPNKPQDYYPQKWVCSCHNKIYNP

SEQ ID NO: 23 - protein, 507 aa, Juniperus ashei
MSMKFMAALAFLALQLIVMAAGEDQSAQIMLDSDTKQYHRSSRNLRKAVHHARHDVAIVFNVEHYGAVGDGKHDSTDAFEKTWNAACNKLSAVFLVPANKKFVVNNLVFYGPCQPHFSFKVDGTIAAYPDPAKWLNSKIWMHFARLTDFNLMGTGVIDGQGNRWWSDQCKTINGRTVCNDKGRPTAIKIDFSKSVTVKELTLTNSPEFHLVFGECDGVKIQGIKIKAPRDSPNTDGIDIFASKRFEIEKCTIGTGDDCVAVGTGSSNITIKDLTCGPGHGMSIGSLGKGNSRSEVSFVHLDGAKFIDTQNGLRIKTWQGGSGLASHITYENVEMINAENPILINQFYCTSAAACKNQRSAVKIQDVTFKNIHGTSATTAAIQLMCSDSVPCSNIKLSNVFLKLTSGKVATCVNKNANGYYTNPLNPSCKSLHPGRTPKELELHQKPTTLLMDEKMGASLNSSPPNCKNKCKGCQPCKPKLIIVHPNQPEDYYPQRWVCSCHNKIYNP

SEQ ID NO: 24 - protein, 384 aa, Hexalectris arizonica
HDVAIVFNVEHHGAVGDGNHDSTDAFEKTWNEACKTLSAVFLVPANKKFVVVNNLVFYGPCQPHFSPKVDGIIAAYPDPVKWKNSKIWMHFARLTDFNLMGTGVIDGQGSKWWSDQCKTVNGRTVCNDKGRPTAIKIDFSKSVTVKELTLMNSPEFHLVFGECDGVKIQGIKIKAPKESPNTDGIDIFGSKRFEIEKCIIGTGDDCVAIGTGSSNITITDLTCGPGHGMSIGSLGKGNSRSEVSFVHLDGAKFIDTQNGLRIKTWQGGSGLASHITYENVEMVNAENPILINQFYCTSAACENQRSAVKIEDVWFKNIHGTSATAAAIQLMCSDSVPCSNIKLSNVVLKLSSGKVAACVNKNANGYYTNPLNPPCKSLHPGPTP

SEQ ID NO: 25 - protein, 225 aa, Juniperus ashei
MARVSELAFLLAATLAISLHMQEAGVVKFDIKNQCGYTVWAAGLPGGGKRLDQGQTWTVNLAAGTASARFWGRTGCTFDASGKGSCQTGDCGGQLSCTVSGAVPATLAEYTQSDQDYYDVSLVDGFNIPLAINPTNAQCTAPACKADINAVCPSELKVDGGCNSACNVFKTDQYCCRNAYVDNCPATNYSKIFKNQCPQAYSYAKDDTATFACASGTDYSIVFCP

SEQ ID NO: 26 - protein, 225 aa, Juniperus rigida
MARVSELALLLVATLAISLHMQEAGAVKFDIKNQCGYTVWAAGLPGGGKRLDQGQTWTLNLAAGTASARFWGRTGCTFDASGKGSCKTGDCGGQLSCTVSGAVPATLAEYTQSDQDYYDVSLVDGFNIPLAINPTNAQCTAPACKADINAVCPSELKVEGGCNSACNVFQTDQYCCRNAYVDNCPATNYSKIFKNQCPQAYSYAKDDTATFACASGTDYSIVFCP

SEQ ID NO: 27 - protein, 224 aa, Cupressus sempervirens
MARVSELALLLVATLAISLHMQEAGAVKFDIKNQCGYTVWAAGLPGGGKRLDQGQTWTVNLAAGTASARFWGRTGCTFDASGKGSCRSGDCGGQLSCTVSGAVPATLAEYTQSDKDYYDVSLVDGFNIPLAINPTNTKCTAPACKADINAVCPSELKVDGGCNSACNVLQTDQYCCRNAYVDNCPATNYSKIFKNQCPQAYSYAKDDTATFACASGTDYSIVFC

SEQ ID NO: 28 - protein, 199 aa, Hexalectris arizonica
VKFDIKNQCGYTVWAAGLPGGGKEFDQGQTWTVNLAAGTASARFWGRTGCTFDASGKGSCRSGDCGGQLSCTVSGAVPATLAEYTQSDQDYYDVSLVDGFNIPLAINPTNTKCTAPACKADINAVCPSELKVDGGCNSACNVLQTDQYCCRNAYVNNCPATNYSKIFKNQCPQAYSYAKDDTATFACASGTDYSIVFCP

SEQ ID NO: 29 - protein, 274 aa, Pinus monticola
MGNSSGNSLMVLLLVLLLVGVTVNAQNCGCASGLCCSQYGYCGSSSAYCGAGCKSGPCSGGGSPSGGGGSVGTIISQSFFNGLAGGAASSCEGKGFYTYNAFIAAANAYSGFGTTGSADVTKRELAAFLANVMHGTGGMCYINERTPPMIYCMSSATWPCASGKSYHGRGPLQLSWNYNYGAAGQSIGFDGVNNPEKVGQDSTISFKTAVWFWMKNSNCHSAITSGQGFGGTIKAINSQECNGGNSGEVNSRVNYYKNICSQLGVDPGANLSCH

SEQ ID NO: 30 - protein, 277 aa, Pseudotsuga menziesii
MGKTGGEKWVMALVLVLLLLGVSVNAQNCGCASGLCCSKYGYCGTTSAYCGTGCRSGPCSSNSGGGSPSGGGGSVGTIISQSIFNGLAGGAASSCEGKGFYTYTAFIKAASAYSGFGTTGSNDVKKRELAAFFANVMHETGGLCYINERNPPMIYCNSSSTWPCASGKSYHGRGPLQLSWNYNYGAAGKSIGFDGLNNPEKVGQDATISFKTAVWFWMNNSNCHSAITGGQGFGATIKAINSGECNGGNSGEVSSRVNYYRKICSQLGVDPGANVSC

SEQ ID NO: 31 - protein, 308 aa, Pinus taeda
MGSRSRILLIGATGYIGRHVAKASLDLGHPTFLLVRESTASSNSEKAQLLESFKASGANIVHGSIDDHASLVEAVKNVDVVISTVGSLQIESQVNIIKAIKEVGTVKRFFPSEFGNDVDNVHAVEPAKSVFEVKAKVRRAIEAEGIPYTYVSSNCFAGYFLRSLAQAGLTAPPRDKVVILGDGNARVVFVKEEDIGTFTIKAVDDPRTLNKTLYLRLPANTLSLNELVALWEKKIDKTLEKAYVPEEEVLKLIADTPFPANISIAISHSIFVKGDQTNFEIGPAGVEASQLYPDVKYTTVDEYLSNFV

SEQ ID NO: 32 - protein, 173 aa, Picea abies
MDSRRLKRSGIVCMVLMSMLMLVVCEDSDNTACLSSLSSCAPYLNATTKPDSSCCSALISVIDKDSQCLCNLLNSDTVKQLGVNVTQAMKMPAECGKNVSATQCNKTATSGGSSVGKTPTSTPPPSSATPSTTTITKSNSNAAASVSVKMFPVAALVFVAVASVLGLKGPCLR

(19) United States
(12) Patent Application Publication
(10) Pub. No.: US 2016/0185831 A1
(43) Pub. Date: Jun. 30, 2016

(54) NUCLEIC ACIDS FOR TREATMENT OF ALLERGIES

(71) Applicants: William Hearl, Damascus, MD (US); Teri Heiland, New Market, MD (US)

(72) Inventors: William Hearl, Damascus, MD (US); Teri Heiland, New Market, MD (US)

(73) Assignee: Immunomic Therapeutics, Inc., Hershey, PA (US)

(21) Appl. No.: 14/407,410

(22) PCT Filed: Jun. 15, 2012
(86) PCT No.: PCT/US12/42552

§ 371 Date: May 6, 2015

(51) Int. Cl. C07K 14/415; A61K 39/35

(52) U.S. Cl. CPC C07K 14/415; A61K 39/35; A61K 2039/53

(57) ABSTRACT

The present invention provides DNA vaccines for the treatment of allergies. The vaccines comprise the coding sequence for one or more allergenic epitopes, and preferably the full protein sequence, of the allergenic protein from which the epitope(s) is derived, fused inframe with the lumenal domain of the lysosomal associated membrane protein (LAMP) and the targeting sequence of LAMP. The vaccines allow for presentation of properly configured three dimensional epitopes for production of an immune response. The vaccines can be multivalent molecules, and/or can be provided as part of a multivalent vaccine containing two or more DNA constructs.

### CROSS-REFERENCE TO RELATED APPLICATIONS

This application relies on the disclosure of, and claims the benefit of the filing date of U.S. provisional patent application No. 61/496,866, filed 14 Jun. 2011, the entire disclosure of which is hereby incorporated herein by reference.

### BACKGROUND OF THE INVENTION

1. Field of the Invention

The present invention relates to the fields of molecular biology and medicine. More specifically, the invention relates to nucleic acids for use as DNA vaccines, and methods of using them to treat subjects suffering from or susceptible to allergic reactions.

2. Discussion of Related Art

Allergy is a hypersensitivity disease characterized by the production of IgE antibodies against an allergen, or allergy-causing molecule. Allergies affect more than 25% of the population. Allergens can enter the body through many routes, including the respiratory tract, skin contact, ingestion, insect bite, or injection of a drug.

Allergy disease management comprises diagnosis and treatment. Allergists diagnose an allergy using a variety of techniques, such as a skin prick test, radioallergosorbent-based techniques, ELISA, or provocation test to demonstrate allergen specific IgE and to identify the allergen source. Treatment of allergy most often falls into two categories: avoidance and dosing with anti-histamines. A third alternative, allergy immunotherapy, requires that the patient receive weekly injections consisting of small amounts of the offending allergens in order to help the immune system reeducate its response to the allergen.

The use and generation of allergen fusion proteins are well known in the art. For example, U.S. Pat. No. 7,566,456 teaches a fusion protein with IgE and IgG binding domains as well as encoding an allergen. Further, WO 97/07218 teaches allergen-anti-CD32 fusion proteins for use in allergy immunotherapy. Neither of these documents, however, teaches how their respective fusion protein interacts with T cells through antigen presentation to induce or modify a Th1 response. Furthermore, there is no theoretical connection between directing the anti-CD32 containing vaccine to dendritic cells to effect a positive induction of Th1 cells. Both of these documents teach a composition that introduces an allergen therapeutically, such that the allergen can be found in the serum as an allergen-fusion protein.

It has been established by Toda et al., 2002 that a T cell epitope of an allergen, in this case a Cry J2 epitope located at amino acid 247-258, can be attached to a fusion protein and be used to conduct allergy-specific immunotherapy. The specific composition described by Toda et al., 2002 is the use of a DNA vaccine encoding the major CD4 T cell epitope of Cry J2, located at amino acids 247-258, attached to class II-associated invariant chain peptide (CLIP). CLIP contains a lysosomal/endosomal trafficking sequence and contains a domain that binds to the peptide binding groove of MHC II. Toda et al., 2002 shows that immunization with the Cry J2 peptide/CLIP DNA vaccine results in priming a mouse to a predominantly Th1 response, characterized by higher IFN-gamma and IgG2a production. However, Toda et al. does not teach the intracellular targeting of the entire protein coding sequence of an allergen useful for conducting allergy-specific immunotherapy.

U.S. Pat. No. 6,982,326 and U.S. Pat. No. 6,090,386 describe nucleic acid sequences coding for the Cryptomeria japonica major pollen allergens Cry J1, Cry J2, Jun s I, and Jun v I, and fragments or peptides thereof. The invention also provides purified Cry J1, Cry J2, Jun s I, and Jun v I, and at least one fragment thereof produced in a host cell transformed with a nucleic acid sequence coding for Cry J1, Cry J2, Jun s I, and Jun v I, or at least one fragment thereof, and fragments of Cry J1, Cry J2, Jun s I, or Jun v I, or at least one fragment thereof, and fragments of Cry J1, Cry J2, Jun s I, or Jun v I prepared synthetically. Cry J1, Cry J2, Jun s I, and Jun v I, and fragments thereof are disclosed as useful for diagnosing, treating, and preventing Japanese cedar pollinosis. The invention also provides isolated peptides of Cry J1 and Cry J2. Peptides within the scope of the invention comprise at least one T cell epitope, or preferably at least two T cell epitopes of Cry J1 or Cry J2. The invention also pertains to modified peptides having similar or enhanced therapeutic properties as the corresponding naturally-occurring allergen or portion thereof but having reduced side effects. Methods of treatment or of diagnosis of sensitivity to Japanese cedar pollens in an individual and therapeutic compositions, and multi-peptide formulations comprising one or more peptides of the invention are also provided. The invention does not teach how to combine the epitopes or allergens into a DNA vaccine with immunostimulatory properties.

U.S. Pat. No. 7,547,440 and U.S. Pat. No. 7,112,329 identify the T-cell epitope site on a Japanese cypress (hinoki) pollen allergen molecule by stimulating a T-cell line established from a patient suffering from Japanese cypress pollen allergy with an overlap peptide covering the primary structure of the Japanese cypress pollen allergen. The peptide is useful in peptide-based immunotherapy for patients with spring tree pollinosis including patients with Japanese cypress pollinosis having cross reactivity with Japanese cypress pollen. The peptide is also useful for diagnosing spring tree pollinosis. The invention is limited to diagnostics and polypeptide delivery of epitopes.

DNA vaccines have been developed as an alternative to traditional whole cell or whole virus vaccines. Generally speaking, DNA vaccines are engineered nucleic acids that include sequences encoding one or more epitopes. The nucleic acids are delivered to cells, typically antigen presenting cells (APCs), the nucleic acids are expressed, and the epitopes present on the expressed proteins are processed in the endosomal/lysosomal compartment, and ultimately presented on the surface of the cell. U.S. Pat. No. 5,633,234 to August et al. discloses and characterizes the endosomal/lysosomal targeting sequence of the lysosomal-associated membrane protein (LAMP). This patent identifies critical residues in the C-terminal region of the protein, which are necessary for targeting of the protein to the endosomal/lysosomal compartment. The patent discloses that fusion of antigenic peptides to the C-terminal LAMP targeting sequence can provide enhanced processing and presentation of epitopes for generation of an immune response.

In addition, U.S. patent application publication number 2004/0157307 to Harris et al. discloses the use of the LAMP lumenal domain as a “trafficking domain” to direct chimeric proteins expressed from DNA vaccines through one or more cellular compartments/organelles, such as through the lysosomal vesicular pathway. The chimeric proteins include the lumenal domain of a LAMP polypeptide, an antigenic domain comprising a peptide epitope sequence previously identified and selected from an antigen protein, a transmembrane domain, and an endosomal/lysosomal targeting sequence.

DNA vaccines have been proposed as a treatment of allergic disease (Raz et al., 1996; Hartl et al., 2004; Hsu et al., 1996; Crameri 2007; Weiss et al., 2006). The underlying rationale is that allergen protein encoded by a DNA vaccine will preferentially activate the allergen-specific Th1 cellular response with the production of interferons by APCs, natural killer (NK), and T cells, rather than the characteristic Th2-type response, such as secretion of IL-4, IL-5, and IL-13, and the formation of IgE by B lymphocytes and the maturation and recruitment of eosinophils in late-phase reactions. However, the mechanisms underlying the differential induction of the Th1 and Th2 T-cell phenotypes appear to involve a large number of factors, such as unique properties of the bacterial DNA of vaccine preparations, e.g., unmethylated and CpG DNA residues, the cytokine milieu elicited by innate immunity, and the cellular trafficking properties of the allergens (Chen et al., 2001; Kaech et al., 2002). No invention or method has successfully addressed the uncertainty of allergy treatment as conducted by delivery of nucleic acids encoding an allergen. Thus, to date such a method of allergy treatment has not been enabled. In addition, administration of DNA vaccines for the treatment of allergic disease has resulted in the secretion of the allergen peptide into the extracellular environment, potentially leading to accidental induction of an allergic response through activation of IgE.

### SUMMARY OF THE INVENTION

The present invention provides nucleic acids (also referred to herein as “constructs”) that encode allergenic proteins, allergenic polypeptides, and allergenic peptides. The nucleic acids are designed for delivery to immune cells and production of allergenic proteins, polypeptides, and peptides within those cells. The encoded proteins, polypeptides, and peptides have targeting sequences for targeting of the proteins to the MHC-II compartment for processing and display of one or more epitopes, resulting in an immune response to the epitope(s). In general, the nucleic acids comprise the following domains, which correlate to the respective domains of the encoded protein: a signal sequence domain; an intra-organelle stabilizing domain; an allergen domain; a transmembrane domain; and a cytoplasmic lysosome/endosome targeting domain.

Within the context of the encoded protein, the signal sequence is provided to direct the encoded protein to the endoplasmic reticulum or a lysosome. The intra-organelle stabilizing domain is a sequence that is designed to be proteolytically resistant and to protect the remaining portions of the protein, and in particular the allergen domain, from degradation prior to processing for epitope presentation by the cell. In exemplary embodiments, the intra-organelle stabilizing domain is the lumenal domain of LAMP-1. The allergen domain comprises the sequence of one or more allergenic epitopes that can serve to raise an immune response in an animal in which the epitopes are presented. Typically, the allergen domain comprises one or more allergen proteins, although in embodiments, immunogenic polypeptide or peptide fragments of allergenic proteins can be used. In exemplary embodiments discussed below, the epitope is an epitope of a plant allergen. In the encoded proteins of the invention, the allergen domain does not include a signal peptide, such as the signal peptide(s) naturally occurring as part of the allergen protein(s). The allergen domain can comprise a single allergenic protein, polypeptide, or peptide, or can comprise two or more allergenic proteins, polypeptides, or peptides. Where two or more allergens are present, each allergen can be from the same species/source or one or more can be from one or more different sources. Where two or more allergens are present, they are coordinately expressed to provide an equal number of copies of each coding region in the expressed protein. The transmembrane domain can be any sequence that is suitable for directing insertion and transfer of a protein through a membrane. Many such sequences are known in the art or can be easily designed. The lysosome/endosome targeting domain can be any sequence that is capable of directing the peptide to a lysosome or endosome. Such sequences are known in the art and are exemplified herein by the cytoplasmic tail sequence of LAMP-1.

As mentioned above, in preferred embodiments, the nucleic acids comprise an allergen domain that includes the entire allergenic coding sequence for an allergenic protein, but lacks the coding sequence for the allergen's signal sequence. In some embodiments, the nucleic acids of the invention do not comprise the entire allergenic coding sequence, but instead comprise only a sufficient amount of the coding sequence such that the encoded polypeptide, when expressed, is able to fold to achieve the natural three dimensional structure of at least one epitope present on the polypeptide. As in constructs comprising an entire allergen coding sequence, where less than the entire coding sequence is present, the nucleic acids construct also lacks the coding sequence for a naturally-occurring signal peptide for the allergenic polypeptide or peptide.

In preferred embodiments, the nucleic acid construct comprises the coding sequences for multiple allergenic proteins, polypeptides, and/or peptides in the allergen domain. Each allergen present can be from the same source, each from a different source, or any combination thereof.

The nucleic acids, and thus the encoded proteins, polypeptides, and peptides of the invention can be used in methods of treating subjects, and in particular animal subjects suffering from or potentially developing allergies. In general, a method of treating according to the present invention comprises administering a nucleic acid of the invention to a subject in an amount sufficient to deliver the nucleic acid to one or more immune cells, and preferably to one or more antigen presenting cells (APC) of the immune system. Once delivered, the nucleic acid is expressed, the encoded protein processed inside the cell, and the epitope(s) displayed on the surface of the cell. The method of treating can be considered a method of using the nucleic acids and proteins to provide a therapeutic or prophylactic immune response.

### BRIEF DESCRIPTION OF THE DRAWINGS

FIG. 1 is a schematic representation of a nucleic acid according to one embodiment of the invention in which a single antigen comprising a single epitope is provided in the allergen domain.

FIG. 2 shows a vector map of a nucleic acid according to the invention, in which the allergen domain comprises the CryJ2 allergen (an allergen from C. japonica), but without a signal sequence, inserted between human LAMP N-terminal sequences (SS and ISOD) and human LAMP C-terminal sequences (TM and TG).

FIG. 3 is a schematic representation of a nucleic acid according to an alternative embodiment of the invention, in which multiple epitope sequences of a single allergen are provided in the allergen domain.

FIG. 4 is a schematic representation of a nucleic acid according to an alternative embodiment of the invention, in which multiple different allergen sequences are provided in the allergen domain.

FIG. 5 shows a vector map of a nucleic acid according to the invention in which the allergen domain comprises the allergen sequences (without signal peptides) for the allergens CryJ1 (an allergen from C. japonica) and CryJ2 (an allergen from C. japonica).

FIG. 6A shows a vector map of a nucleic acid that includes three peanut allergens (AraH1, AraH2, and AraH3, all lacking signal sequences) in the allergen domain.

FIG. 6B shows a schematic of the protein encoded by the nucleic acid of FIG. 6A.

FIG. 7 shows a vector map of a nucleic acid according to the present invention, depicting the absence of the naturally-occurring signal sequence for the CryJ1 allergen sequence. This particular construct is used in experiments detailed below to show the importance of removal of the natural signal sequence of allergen sequences.

FIG. 8 shows a vector map of a nucleic acid construct not encompassed by the present invention, in which the CryJ2 allergen is encoded on a plasmid backbone, but in the absence of the SS, IOS, TM, and TG domains. This construct is used as a comparative control in experiments detailed below.

FIG. 9 shows Western blots depicting expression of constructs according to the invention in 293 cells. Panel A shows expression of the CryJ1-CryJ2 combined allergens (see FIG. 5) and the CryJ2 allergen alone (see FIG. 2) in constructs according to the invention, when assayed with anti-CryJ2 antibodies. Panel B shows expression of the CryJ1-CryJ2 combined allergens and the CryJ1 allergen (lacking its native signal sequence; see FIG. 7), when assayed with anti-CryJ1 antibodies. Panel B further shows that expression of the CryJ1 allergen is not detectable in a construct in which the natural signal sequence for the CryJ1 allergen is not removed (vector map not shown).

FIG. 10 shows line graphs depicting the effectiveness of nucleic acid constructs according to the present invention as compared to other constructs comprising allergen sequences. Panel A shows that a significant increase in IgG1 production and detection is seen as a result of administration of the CryJ2-LAMP construct of the invention (see FIG. 2) as compared to a construct comprising a plasmid backbone fused to the CryJ2 coding sequence (see FIG. 8). Panel B shows that a significant increase in IgG2a production and detection is seen as a result of administration of the CryJ2-LAMP construct of the invention (as per Panel A) as compared to a construct comprising a plasmid backbone fused to the CryJ2 coding sequence (as per Panel A).

FIG. 11 depicts bar graphs showing dosing effects of the CryJ2-LAMP construct in mice. Panel A depicts IgG2a detection at 21 days and 28 days post injection of the DNA vaccine at various amounts ranging from 10 ug to 100 ug, as compared to injection of vector DNA alone. Panel B depicts IgG1 detection at 21 days and 28 days post injection of the DNA vaccine at various amounts ranging from 10 ug to 100 ug, as compared to injection of vector DNA alone.

FIG. 12 depicts bar graphs showing the effect on induction of IL-4 and IFN-gamma in mouse spleen cultures treated with the CryJ2-LAMP construct of the invention as compared to vector alone. Panel A shows the effect of IL-4. Panel B shows the effect of IFN-gamma.

FIG. 13 depicts line graphs showing the effectiveness of immunization of previously sensitized mice with the CryJ2-LAMP DNA vaccine. Panel A shows IgG1 titers over time. Panel B shows IgG2a titers over time.

FIG. 14 depicts bar graphs showing induction of IFN-g (Panel A) and IL-4 (Panel B) in mouse spleen cell cultures.

FIG. 15 depicts a bar graph showing quantitation of circulating CryJ2 protein in immunized mice.

FIG. 16 depicts bar graphs of guinea pig data, showing IgG1 detection (Panel A) and IgG2 detection (Panel B) for guinea pigs immunized with the CryJ2-LAMP construct and challenged with recombinant CryJ2.

FIG. 17 depicts a bar graph showing the Anti-CryJ2 response in New Zealand white rabbits immunized with CryJ2-LAMP DNA vaccine during an 85 day toxicology GLP safety study.

FIG. 18 depicts a Western blot showing co-expression of peanut allergens H1, H2, and H3 from a construct according to the present invention.

### DETAILED DESCRIPTION OF EXEMPLARY EMBODIMENTS

Reference will now be made in detail to various exemplary embodiments of the invention. It is to be understood that the following discussion of exemplary embodiments is not intended as a limitation on the invention, as broadly disclosed herein. Rather, the following discussion is provided to give the reader a more detailed understanding of certain aspects and features of the invention. The practice of the present invention employs, unless otherwise indicated, conventional molecular biology, microbiology, and recombinant DNA techniques within the skill of those in the art. Such techniques are explained fully in the literature known to the ordinary artisan in these fields, and thus need not be detailed herein. Likewise, practice of the invention for medical treatment follows standard protocols known in the art, and those protocols need not be detailed herein.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. It is thus to be understood that, where a range of values is presented, each value within that range, and each range falling within that range, is inherently recited as well, and that the avoidance of a specific recitation of each and every value and each and every possible range of values is not an omission of those values and ranges, but instead is a convenience for the reader and for brevity of this disclosure.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “an allergen” includes a plurality of such allergens and reference to “the sample” includes reference to one or more samples and equivalents thereof known to those skilled in the art, and so forth. Furthermore, the use of terms that can be described using equivalent terms include the use of those equivalent terms. Thus, for example, the use of the term “subject” is to be understood to include the terms “animal”, “human”, and other terms used in the art to indicate one who is subject to a medical treatment.

As used herein, the term “comprising” is intended to mean that the constructs, compositions, and methods include the recited elements and/or steps, but do not exclude other elements and/or steps. “Consisting essentially of”, when used to define constructs, compositions, and methods, means excluding other elements and steps of any essential significance to the recited constructs, compositions, and methods. Thus, a composition consisting essentially of the elements as defined herein would not exclude trace contaminants from the isolation and purification method and pharmaceutically acceptable carriers, such as phosphate buffered saline, preservatives, and the like. “Consisting of” means excluding more than trace elements of other ingredients and substantial method steps for administering the compositions of this invention. Embodiments defined by each of these transition terms are within the scope of this invention.

A “chimeric DNA” is an identifiable segment of DNA within a larger DNA molecule that is not found in association with the larger molecule in nature. Thus, when the chimeric DNA encodes a protein segment, the segment coding sequence will be flanked by DNA that does not flank the coding sequence in any naturally occurring genome. In the case where the flanking DNA encodes a polypeptide sequence, the encoded protein is referred to as a “chimeric protein” (i.e., one having non-naturally occurring amino acid sequences fused together). Allelic variations or naturally occurring mutational events do not give rise to a chimeric DNA or chimeric protein as defined herein.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the term “peptide” refers to a compound of two or more subunit amino acids, amino acid analogs, or peptidomimetics. The subunits may be linked by peptide bonds or by other bonds (e.g., as esters, ethers, and the like). The term “peptide” is used herein generically to refer to peptides (i.e., polyamino acids of from 2 to about 20 residues), polypeptides (i.e., peptides of from about 20 residues to about 100 residues), and proteins (i.e., peptides having about 100 or more residues).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. While the term “protein” encompasses the term “polypeptide”, a “polypeptide” may be a less than a full-length protein.

The term “allergen” refers to any naturally occurring protein or mixtures of proteins that have been reported to induce allergic, i.e., IgE mediated reactions upon their repeated exposure to an individual. An allergen is any compound, substance, or material that is capable of evoking an allergic reaction. Allergens are usually understood as a subcategory of antigens, which are compounds, substances, or materials capable of evoking an immune response. For carrying out the invention, the allergen may be selected, among other things, from natural or native allergens, modified natural allergens, synthetic allergens, recombinant allergens, allergoids, and mixtures or combinations thereof. Of particular interest are allergens that are capable of causing an IgE-mediated immediate type hypersensitivity.

Examples of naturally occurring allergens include pollen allergens (e.g., tree, weed, herb and grass pollen allergens), mite allergens (from e.g. house dust mites and storage mites), insect allergens (e.g., inhalant, saliva- and venom origin allergens), animal allergens from e.g. saliva, hair and dander from animals (e.g. dog, cat, horse, rat, mouse, etc.), fungi allergens and food allergens. The allergen may be in the form of an allergen extract, a purified allergen, a modified allergen or a recombinant allergen or a recombinant mutant allergen, an allergen fragment above 30 amino acids or any combination thereof.

In terms of their chemical or biochemical nature, allergens can represent native or recombinant proteins or peptides, fragments or truncated versions of native or recombinant proteins or peptides, fusion proteins, synthetic compounds (chemical allergens), synthetic compounds that mimic an allergen, or chemically or physically altered allergens, such as allergens modified by heat denaturation.

The classification of an allergen as a major allergen can be subject to several tests. An allergen is commonly classified as a major allergen if at least 25% of patients show strong IgE binding (score 3) and at least moderate binding (score 2) from 50% of the patients, the binding being determined by an CRIE (Crossed Radio Immune Electrophoresis) (CRIE Strong binding, i.e., visible IgE-binding on an X-ray film after one day; CRIE Moderate binding, i.e., binding after 3 days; CRIE Weak binding, i.e., binding after 10 days). Strong IgE binding from at least 10% of the patients classifies the allergen as an Intermediate allergen and clearly specific binding from less than 10% of the patients classifies it as a Minor allergen. Other methods may also be used in determining the IgE binding of for instance IgE-blots.

An “epitope” is a structure, usually made up of a short peptide sequence or oligosaccharide, that is specifically recognized or specifically bound by a component of the immune system. T-cell epitopes have generally been shown to be linear oligopeptides. Two epitopes correspond to each other if they can be specifically bound by the same antibody. Two epitopes correspond to each other if both are capable of binding to the same B cell receptor or to the same T cell receptor, and binding of one antibody to its epitope substantially prevents binding by the other epitope (e.g., less than about 30%, preferably, less than about 20%, and more preferably, less than about 10%, 5%, 1%, or about 0.1% of the other epitope binds).

As used herein, two nucleic acid coding sequences “correspond” to each other if the sequences or their complementary sequences encode the same amino acid sequences.

As used herein, a polynucleotide or polynucleotide region (or a polypeptide or polypeptide region) which has a certain percentage (for example, at least about 50%, at least about 60%, at least about 70%, at least about 80%, at least about 85%, at least about 90%, at least about 95%, at least about 99%) of “sequence identity” to another sequence means that, when maximally aligned, manually or using software programs routine in the art, that percentage of bases (or amino acids) are the same in comparing the two sequences.

Two nucleotide sequences are “substantially homologous” or “substantially similar” when at least about 50%, at least about 60%, at least about 70%, at least about 75%, and preferably at least about 80%, and most preferably at least about 90 or 95% of the nucleotides match over the defined length of the DNA sequences. Similarly, two polypeptide sequences are “substantially homologous” or “substantially similar” when at least about 40%, at least about 50%, at least about 60%, at least about 66%, at least about 70%, at least about 75%, and preferably at least about 80%, and most preferably at least about 90 or 95% or 98% of the amino acid residues of the polypeptide match over a defined length of the polypeptide sequence. Sequences that are substantially homologous can be identified by comparing the sequences using standard software available in sequence data banks Substantially homologous nucleic acid sequences also can be identified in a Southern hybridization experiment under, for example, stringent conditions as defined for that particular system. Defining appropriate hybridization conditions is within the skill of the art. For example, stringent conditions can be: hybridization at 5×SSC and 50% formamide at 42° C., and washing at 0.1×SSC and 0.1% sodium dodecyl sulfate at 60° C.

“Conservatively modified variants” of domain sequences also can be provided. With respect to particular nucleic acid sequences, the term conservatively modified variants refers to those nucleic acids that encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer, et al., 1991, Nucleic Acid Res. 19: 5081; Ohtsuka, et al., 1985, J. Biol. Chem. 260: 2605-2608; Rossolini et al., 1994, Mol. Cell. Probes 8: 91-98).

The term “biologically active fragment”, “biologically active form”, “biologically active equivalent”, and “functional derivative” of a wild-type protein, means a substance that possesses a biological activity that is at least substantially equal (e.g., not significantly different from) the biological activity of the wild type protein as measured using an assay suitable for detecting the activity. For example, a biologically active fragment comprising a trafficking domain is one which can co-localize to the same compartment as a full length polypeptide comprising the trafficking domain.

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

A “replicon” is any genetic element (e.g., plasmid, chromosome, virus) that functions as an autonomous unit of DNA replication in vivo.

As used herein, a “viral vector” refers to a virus or viral particle that comprises a polynucleotide to be delivered into a host cell, either in vivo, ex vivo, or in vitro. Examples of viral vectors include, but are not limited to, adenovirus vectors, adeno-associated virus vectors, retroviral vectors, and the like. In aspects where gene transfer is mediated by an adenoviral vector, a vector construct refers to the polynucleotide comprising the adenovirus genome or part thereof, and a selected, non-adenoviral gene, in association with adenoviral capsid proteins.

As used herein, a “nucleic acid delivery vector” is a nucleic acid molecule that can transport a polynucleotide of interest into a cell. Preferably, such a vector comprises a coding sequence operably linked to an expression control sequence. However, a polynucleotide sequence of interest does not necessarily comprise a coding sequence. For example, in one aspect, a polynucleotide sequence of interest is an aptamer which binds to a target molecule. In another aspect, the sequence of interest is a complementary sequence of a regulatory sequence that binds to a regulatory sequence to inhibit regulation of the regulatory sequence. In still another aspect, the sequence of interest is itself a regulatory sequence (e.g., for titrating out regulatory factors in a cell).

As used herein, a “nucleic acid delivery vehicle” is defined as any molecule or group of molecules or macromolecules that can carry inserted polynucleotides into a host cell (e.g., such as genes or gene fragments, antisense molecules, ribozymes, aptamers, and the like) and that occurs in association with a nucleic acid delivery vector as described above.

As used herein, “nucleic acid delivery” or “nucleic acid transfer” refers to the introduction of an exogenous polynucleotide (e.g., such as a transgene) into a host cell, irrespective of the method used for the introduction. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” to an expression control sequence when the expression control sequence controls and regulates the transcription of that DNA sequence.

As used herein, “coding sequence” is a sequence which is transcribed and translated into a polypeptide when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., mammalian) DNA, and even synthetic DNA sequences. A polyadenylation signal and transcription termination sequence will usually be located 3′ to the coding sequence.

As used herein, a “genetic modification” refers to any addition to or deletion or disruption of a cell's normal nucleotide sequence. Any method that can achieve the genetic modification of APCs are within the spirit and scope of this invention. Art recognized methods include viral mediated gene transfer, liposome mediated transfer, transformation, transfection and transduction, e.g., viral-mediated gene transfer such as the use of vectors based on DNA viruses such as adenovirus, adeno-associated virus and herpes virus, as well as retroviral based vectors.

As used herein, “the lysosomal/endosomal compartment” refers to membrane-bound acidic vacuoles containing LAMP molecules in the membrane, hydrolytic enzymes that function in antigen processing, and MHC class II molecules for antigen recognition and presentation. This compartment functions as a site for degradation of foreign materials internalized from the cell surface by any of a variety of mechanisms including endocytosis, phagocytosis, and pinocytosis, and of intracellular material delivered to this compartment by specialized autolytic phenomena (see, for example, de Duve, Eur. J. Biochem. 137: 391, 1983). The term “endosome” as used herein encompasses a lysosome.

As used herein, a “lysosome-related organelle” refers to any organelle that comprises lysozymes and includes, but is not limited to, MIIC, CIIV, melanosomes, secretory granules, lytic granules, platelet-dense granules, basophil granules, Birbeck granules, phagolysosomes, secretory lysosomes, and the like. Preferably, such an organelle lacks mannose 6-phosphate receptors and comprises LAMP, but might or might not comprise an MHC class II molecule. For reviews, see, e.g., Blott and Griffiths, Nature Reviews, Molecular Cell Biology, 2002; Dell'Angelica, et al., The FASEB Journal 14: 1265-1278,2000.

As used herein a “LAMP polypeptide” refers to LAMP-1, LAMP-2, CD63/LAMP-3, DC-LAMP, or any lysosomal associated membrane protein, or homologs, orthologs, variants (e.g., allelic variants) and modified forms (e.g., comprising one or more mutations, either naturally occurring or engineered). In one aspect, a LAMP polypeptide is a mammalian lysosomal associated membrane protein, e.g., such as a human or mouse lysosomal associated membrane protein. More generally, a “lysosomal membrane protein” refers to any protein comprising a domain found in the membrane of an endosomal/lysosomal compartment or lysosome-related organelle and which further comprises a lumenal domain.

As used herein, “targeting” denotes the polypeptide sequence that directs a chimeric protein of the invention to a preferred site, such as a cellular organelle or compartment where antigen processing and binding to MHC II occurs. As such, a “targeting domain” refers to a series of amino acids that are required for delivery to a cellular compartment/organelle. Preferably, a targeting domain is a sequence that binds to an adaptor or AP protein (e.g., such as an AP1, AP2, or AP3 protein). Exemplary targeting domain sequences are described in Dell'Angelica, 2000, for example.

As used herein, in vivo nucleic acid delivery, nucleic acid transfer, nucleic acid therapy, and the like, refer to the introduction of a vector comprising an exogenous polynucleotide directly into the body of an organism, such as a human or non-human mammal, whereby the exogenous polynucleotide is introduced into a cell of such organism in vivo.

As used herein, the term in situ refers to a type of in vivo nucleic acid delivery in which the nucleic acid is brought into proximity with a target cell (e.g., the nucleic acid is not administered systemically). For example, in situ delivery methods include, but are not limited to, injecting a nucleic acid directly at a site (e.g., into a tissue, such as a tumor or heart muscle), contacting the nucleic acid with cell(s) or tissue through an open surgical field, or delivering the nucleic acid to a site using a medical access device such as a catheter.

As used herein, the terms “isolated” and “purified” are used at times interchangeably to mean separated from constituents, cellular and otherwise, in which the polynucleotide, peptide, polypeptide, protein, antibody, or fragments thereof, are normally associated with in nature. For example, with respect to a polynucleotide, an isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. Furthermore, the terms “isolated” and “purified” do not imply total isolation and total purity. These terms are used to denote both partial and total purity from some or all other substances naturally found in association with the polynucleotide, etc. Thus, these terms can mean isolation or purification from one naturally associated substance (e.g., isolation or purification of DNA from RNA), isolation or purification from other substances of the same general class of molecule (e.g., a particular protein showing 20% purity as compared to all proteins in a sample), or any combination. Isolation and purification can mean any level from about 1% to about 100%, including 100%. As such, an “isolated” or “purified” population of cells is substantially free of cells and materials with which it is associated in nature. By substantially free or substantially purified APCs is meant at least 50% of the population of cells are APCs, preferably at least 70%, more preferably at least 80%, and even more preferably at least 90% free of non-APCs cells with which they are associated in nature. Of course, those of skill in the art will recognize that all specific values, including fractions of values, are encompassed within these ranges without the need for each particular value to be listed herein. Each value is not specifically disclosed for the sake of brevity; however, the reader is to understand that each and every specific value is inherently disclosed and encompassed by the invention.

As used herein, a “target cell” or “recipient cell” refers to an individual cell or cell which is desired to be, or has been, a recipient of exogenous nucleic acid molecules, polynucleotides, and/or proteins. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

The term “antigen presenting cell” or “APC” as used herein intends any cell that presents on its surface an antigen in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster antigen presenting cells.

As used herein an “engineered antigen-presenting cell” refers to an antigen-presenting cell that has a non-natural molecular moiety on its surface. For example, such a cell may not naturally have a co-stimulator on its surface or may have additional artificial co-stimulator in addition to natural co-stimulator on its surface, or may express a non-natural class II molecule on its surface.

As used herein, the term “immune effector cells” refers to cells that are capable of binding an antigen and that mediate an immune response. These cells include, but are not limited to, T cells, B cells, monocytes, macrophages, NK cells, and cytotoxic T lymphocytes (CTLs), for example CTL lines, CTL clones, and CTLs from tumor, inflammatory, or other infiltrates.

As used herein, the terms “subject” and “patient” are used interchangeably to indicate an animal for which the present invention is directed. The term animal is to be understood to include humans and non-human animals; where a distinction between the two is desired, the terms human and/or non-human animal are used. In embodiments, the subject or patient is a vertebrate, preferably a mammal, more preferably a human. Mammals include, but are not limited to, murines, simians, humans, farm animals (e.g., bovines, ovines, porcines), sport animals (e.g. equines), and pets (e.g., canines and felines).

Clinical allergy symptoms are known to those of skill in the art, and an exhaustive listing herein is not required. Non-limiting examples include rhinitis, conjunctivitis, asthma, urticaria, eczema, which includes reactions in the skin, eyes, nose, upper and lower airways with common symptoms such as redness and itching of eyes and nose, itching and runny nose, coaching, wheezing, shortness of breath, itching, and swelling of tissue.

Examples of “immunological in vivo tests” are Skin Prick Test (SPT), Conjunctival Provocation Test (CPT), Bronchial Challenge with Allergen (BCA), and various clinical tests in which one or more allergy symptoms is monitored. See, for example, Haugaard et al., J Allergy Clin Immunol, Vol. 91, No. 3, pp 709-722, March 1993.

As used herein, the term “pharmaceutically acceptable carrier” encompasses any of the standard pharmaceutical carriers known in the art, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

As used herein, a “therapeutically effective amount” is used herein to mean an amount sufficient to prevent, correct, and/or normalize an abnormal physiological response. In one aspect, a “therapeutically effective amount” is an amount sufficient to reduce by at least about 30 percent, more preferably by at least 50 percent, most preferably by at least 90 percent, a clinically significant feature of pathology, such as for example, size of a tumor mass, antibody production, cytokine production, fever or white cell count, or level of histamine.

An “antibody” is any immunoglobulin, including antibodies and fragments thereof, that binds a specific epitope. The term encompasses polyclonal, monoclonal, and chimeric antibodies (e.g., bispecific antibodies). An “antibody combining site” is that structural portion of an antibody molecule comprised of heavy and light chain variable and hypervariable regions that specifically binds antigen. Exemplary antibody molecules are intact immunoglobulin molecules, substantially intact immunoglobulin molecules, and those portions of an immunoglobulin molecule that contains the paratope, including Fab, Fab′, F(ab′)₂, and F(v) portions, which portions are preferred for use in the therapeutic methods described herein.

The term “oromucosal administration” refers to a route of administration where the dosage form is placed under the tongue or anywhere else in the oral cavity to allow the active ingredient to come in contact with the mucosa of the oral cavity or the pharynx of the patient in order to obtain a local or systemic effect of the active ingredient. An example of an oromucosal administration route is sublingual administration. The term “sublingual administration” refers to a route of administration where a dosage form is placed underneath the tongue in order to obtain a local or systemic effect of the active ingredient. As used herein, the term “intradermal delivery” means delivery of the vaccine to the dermis in the skin. However, the vaccine will not necessarily be located exclusively in the dermis. The dermis is the layer in the skin located between about 1.0 and about 2.0 mm from the surface in human skin, but there is a certain amount of variation between individuals and in different parts of the body. In general, it can be expected to reach the dermis by going 1.5 mm below the surface of the skin. The dermis is located between the stratum corneum and the epidermis at the surface and the subcutaneous layer below. Depending on the mode of delivery, the vaccine may ultimately be located solely or primarily within the dermis, or it may ultimately be distributed within the epidermis and the dermis.

As used herein, the term “prevent” in the context of allergy immunotherapy, allergy treatment, or other terms that describe an intervention designed for an allergy patient, means the prevention of an IgE response in at least 20% of all patients. The term “prevent” does not mean total prevention from developing an IgE mediated disease in all patients, and such a definition is outside the scope of the present invention for treating allergy through a mechanism that reduces allergy symptoms, and is inconsistent with the use of the term in the art. It is well known to those skilled in the art of allergy immunotherapy that allergy treatments are not 100% effective in 100% of patients, and as such an absolute definition of “prevent” does not apply within the context of the present invention. The art-recognized concept of prevention is contemplated by the present invention.

The present invention provides polynucleic acids, polyaminoacids, and methods of treating subjects in need of the polynucleic acids and polyaminoacids. Broadly speaking, the polynucleic acids can be thought of as nucleic acid (e.g., DNA, RNA) vaccines for the intracellular production of allergenic sequences (polyaminoacids) that elicit a protective immune response within the body of the subject to whom the polynucleic acid is administered. The polynucleic acids, when administered, preferentially evoke a cell-mediated immune response via the MHC-II pathway and production of IgG antibodies by activating an allergen-specific T-helper type 1 (Th1) cellular response with the production of interferons by APCs, NK cells, and T cells rather than a Th2-type response, which involves production of IgE antibodies, granulocytes (e.g., eosinophils), and other substances. To an extent, both an MHC-II and an MHC-I response can be generated; however, the invention provides a response that is primarily or substantially an MHC-II response. Preferably, the nucleic acids do not encode an antibiotic resistance gene.

The invention is based, at least in part, on the recognition that a combination of certain structural, and thus functional, elements provides advantageous properties to the nucleic acid vaccines and the encoded allergens, and allows for allergy treatment methods that satisfy unmet needs in the art. In the various embodiments of the invention, which are intended to be understood as standing alone as independent embodiments and as embodiments that combine two or more features of the independent embodiments, the combinations include the use of a lysosomal trafficking domain to direct allergen amino acid sequences to lysosomes with MHC II proteins. Doing so allows for predominantly an IgG response as opposed to an IgE response to the allergen sequences. Yet further, independent embodiments or combinations of embodiments provide constructs containing a sufficient length of a nucleic acid sequence to encode an amino acid sequence that provides a naturally-occurring three-dimensional structure of an epitope. In preferred embodiments, the nucleic acid sequence provides/encodes the full-length allergen coding sequence, but which lacks any naturally-occurring signal peptide sequence associated with the allergen sequence. In other embodiments, the nucleic acid sequence encodes at least one allergenic region of an allergen, but not the full-length allergen protein (and also lacking the signal sequence, if one was naturally present). Although it is recognized in the art that an immune response can be generated against the primary sequence of an epitope, the present invention recognizes that nucleic acid vaccines for the production of an MHC-II immune response to encoded epitopes preferably uses nucleic acid constructs that encode enough sequence data to produce a correct three-dimensional peptide structure in the region comprising an allergenic epitope, at least at the time when the allergenic sequence is delivered to a lysosome for processing. While not being limited to any particular molecular theory, it is believed that delivery of a properly three-dimensionally folded protein, polypeptide, or peptide to an endosome improves processing and presentation of allergenic epitopes for an immune response.

As yet another example of an embodiment that can be implemented, alone or as part of a combination of embodiments, the expression of multiple allergens from a single construct is provided. To date, it has not been shown that a nucleic acid vaccine that is protective against an allergen can be effectively produced and used. The present invention not only provides an effective nucleic acid vaccine against an allergen, but further provides an effective nucleic acid vaccine against multiple allergens at the same time. The allergens can be allergens from the same source (e.g., a single plant), or can be allergens from two or more sources (e.g., a tree, a flower, a food, etc.). As above, the full-length allergen sequences can be used (lacking any naturally-associated signal sequence for the allergen), or allergenic portions can be used. In constructs comprising multiple allergen sequences, any mixture of full-length or truncated allergen sequences can be used. Further, as with other embodiments, it is preferred that naturally-occurring signal sequence for each allergen sequence be removed (i.e., the naturally-occurring signal sequences for each allergen sequence are not present in the constructs).

Although the use of signal sequences for the independent allergenic sequences within the allergen domain has been found to be detrimental to the function of the nucleic acid construct, it has been found that the use of signal sequence region or domain within the nucleic acid vaccine constructs is an important feature. As such, in embodiments, the nucleic acid vaccine includes at least one signal sequence within the signal sequence domain to direct the encoded peptide to and through a membrane. Although the amino acid sequence of the signal sequence may vary from construct to construct, and any known signal sequence can be selected, it has been found that in preferred embodiments, the signal sequence is present and provided in-frame with the coding sequence of the allergen sequence(s). The use of a single signal sequence is adequate to direct the entire encoded chimeric protein to and through a membrane. As such, signal sequences for each allergen sequence are not necessary and, in fact, have been found to be detrimental to proper localization, processing, and expression of allergen epitopes on immune cell surfaces.

And further, in specific embodiments and in combinations of embodiments, it has been found that sequestration, or physical protection, of allergen sequences during the transfer of the polypeptide from the cytoplasm to the endosome, including the time in the endosome prior to cleavage of the polypeptide into units for presentation at the cell surface, can be an important factor in providing a useful nucleic acid vaccine according to the invention. As such, in general, the invention includes a construct that comprises an intra-organelle stabilizing domain (IOSD) to protect allergen sequences.

The nucleic acid of the invention comprises at least the following domains: a signal sequence domain; an intra-organelle stabilizing domain; an allergen domain (which can comprise a single allergen or two or more allergens, each comprising one or more allergenic epitopes); a transmembrane domain; and a cytoplasmic lysosome/endosome targeting domain. The various domains are present on a single chimeric or engineered nucleic acid. The various domains can be combined in any linear order using techniques known and widely practiced in the art. In preferred embodiments, the domains are combined and arranged such that they comprise a single open reading frame encoding a chimeric protein, the open reading frame being operably linked to transcriptional elements sufficient for expression of the chimeric protein. The nucleic acid thus can be an expression vector, such as a plasmid, phagemid, viral vector, or the like. Preferably, the nucleic acid comprises transcriptional elements suitable for expression in mammalian cells, such as human cells. Such expression vector elements and expression vectors are known and widely used in the art, as exemplified by U.S. patent application publication number 2004/0157307, which is incorporated herein by reference. A non-limiting example of a plasmid backbone for use in creating nucleic acid constructs according to the invention is referred to at times herein as a “pITI” plasmid, the sequence of which is provided as SEQ ID NO:1.

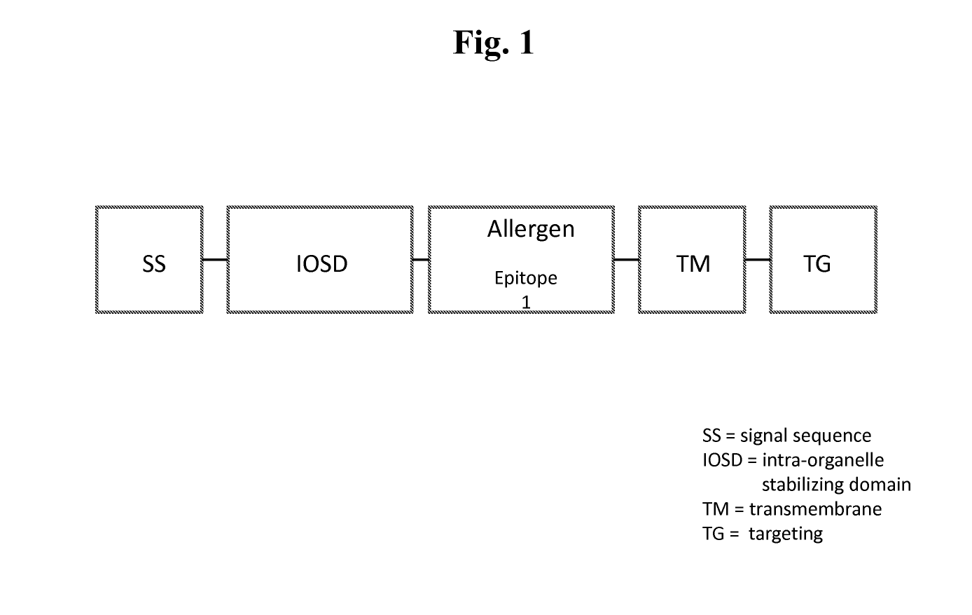

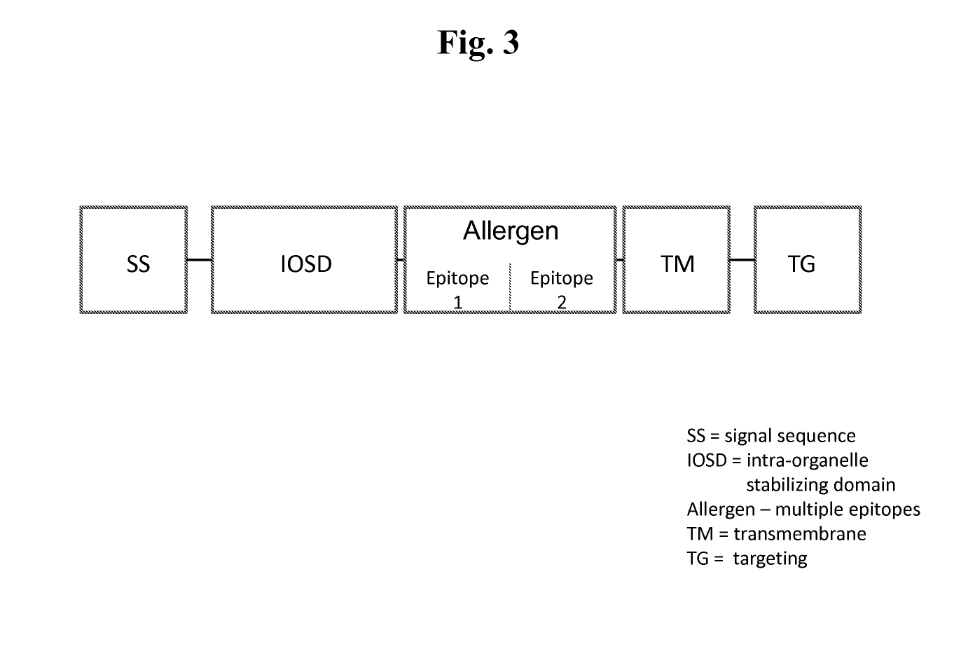

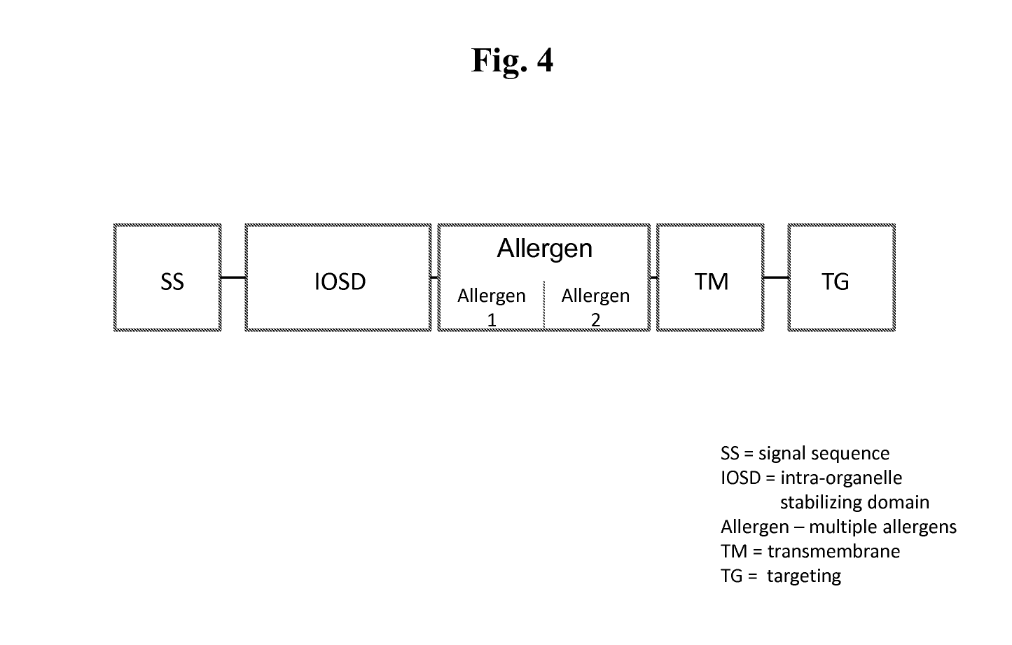

Three exemplary configurations of the nucleic acid of the invention are depicted schematically in FIGS. 1, 3, and 4, respectively. FIG. 1 shows a sequential arrangement of domains in which a single allergen comprising a single epitope is included in the encoded chimeric protein. FIG. 3 shows a sequential arrangement of domains in which multiple different epitopes of a single allergen are included in the encoded chimeric protein within the allergen domain. The two epitopes are arranged such that they are in the same reading frame and are thus both produced as part of the chimeric protein. Those of skill in the art will immediately recognize that three or more epitopes can be provided in the same reading frame within the epitope domain using standard molecular biology techniques. FIG. 4 shows a sequential arrangement of domains in which two different allergens are present in the allergen domain. Of course, the skilled artisan will recognize that each allergen sequence can contain one or multiple allergenic epitopes. Based on these three schematic representations of embodiments of the nucleic acids of the invention, the reader will immediately recognize that any number of allergens, from any number of sources, and containing any number of epitopes, can be included within the allergen domain, and can be linked in-frame using standard molecular biology techniques.

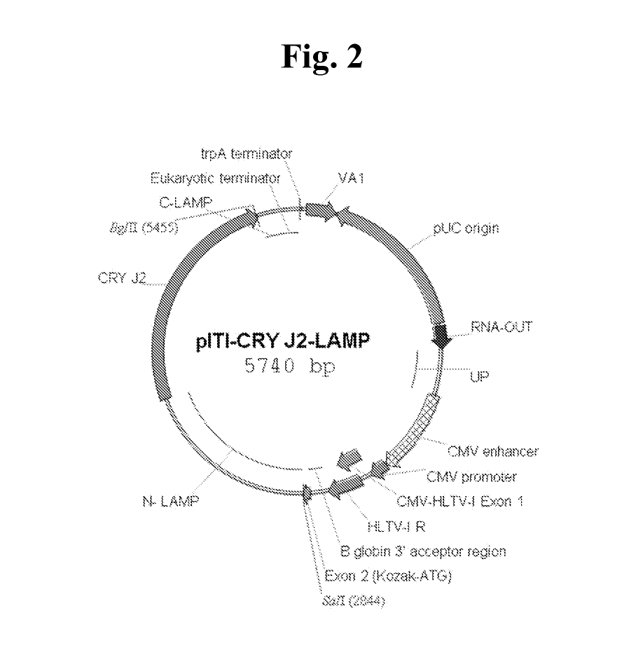

FIG. 2 depicts a vector map of a nucleic acid according to one embodiment of the invention (“pITI-CRY J2-LAMP”; also referred to herein at times as “CRYJ2-LAMP”), which generally relates to the embodiment of the invention depicted schematically in FIG. 1. The vector or delivery vehicle includes a plasmid backbone with a pUC origin of replication and various transcription and expression elements for production of the encoded protein. More specifically, it includes the sequence of the pITI backbone (SEQ ID NO:1). It is to be noted that the nucleic acid construct does not include an antibiotic resistance gene, in accordance with preferred embodiments of the invention. The nucleic acid further comprises sequences for the encoded protein, which comprises an N-terminal region of the human LAMP protein, which includes a signal sequence and an intra-organelle stabilizing domain. The nucleic acid further provides sequences for the encoded protein that comprises the CryJ2 allergen sequence (lacking its signal sequence) fused in-frame to the N-terminal region of the LAMP protein. The nucleic acid further includes sequences encoding a portion of the C-terminal region of the human LAMP protein, which includes a transmembrane region and a targeting region. The coding region for the CRY J2-LAMP chimeric protein sequence is provided as SEQ ID NO:2. The amino acid sequence for the CRY J2-LAMP chimeric protein is provided as SEQ ID NO:3.

In exemplary embodiments, the invention also relates to nucleic acid constructs for the delivery and expression of other allergens of C. japonica, including the CryJ1 allergen. Using the same plasmid backbone, a pITI-CRYJ1-LAMP construct has been created. The chimeric protein can elicit an MHC II type immune response. The coding region for the pITI-CRYJ1-LAMP construct is presented as SEQ ID NO:4. The amino acid sequence for the CRY J1-LAMP chimeric protein is provided as SEQ ID NO:5.

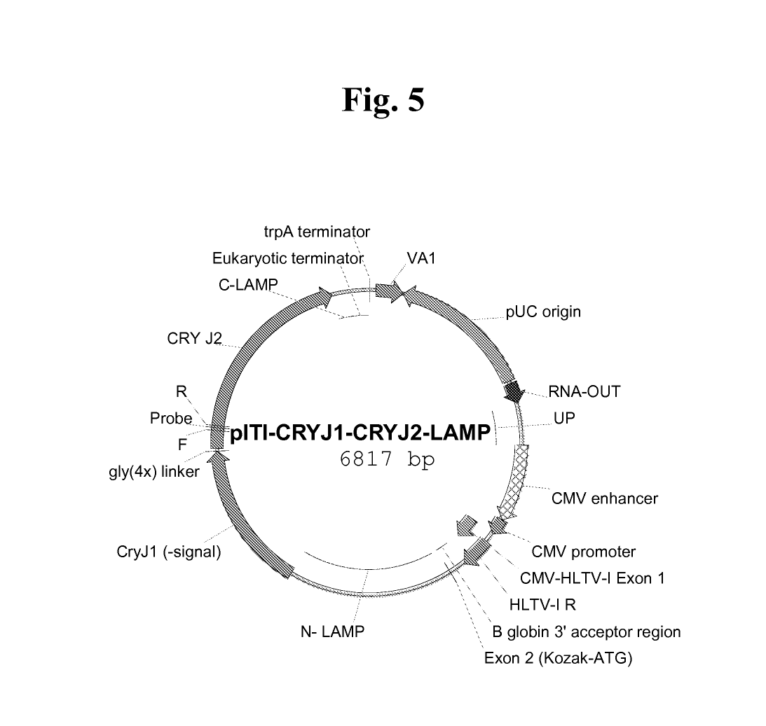

As shown in FIGS. 3 and 4, the allergen domain can include an allergen having multiple allergenic epitopes, or can include multiple allergens (each having one or more allergenic epitopes). FIG. 5 depicts a vector map of a particular exemplary embodiment of a nucleic acid construct in which the allergen domain includes two allergenic sequences. In this exemplary embodiment, the allergen domain contains the CryJ1 and CryJ2 allergens (each lacking its natural signal sequence) of C. japonica fused in-frame and fused at the N-terminal end with a LAMP signal sequence domain and intra-organelle stabilizing domain. The CryJ1-CryJ2 sequences are also fused at the C-terminal end with a LAMP transmembrane domain and targeting domain. The full nucleotide sequence of the coding region for the chimeric protein is presented as SEQ ID NO:6. The full amino acid sequence of the encoded chimeric protein is presented as SEQ ID NO:7, in which: residues 1-27 represent the signal sequence for the chimeric protein; residues 28-380 represent the intra-organelle stabilizing domain (sequence taken from human LAMP); residues 381 and 382 represent a linker; residues 383-735 represent the coding region of the CryJ1 (without its signal sequence); residues 736-741 represent a linker region; residues 742-1232 represent the coding region for the CryJ2 allergen; residues 1233-1234 represent a linker region; residues 1235-1258 represent the transmembrane and targeting domain; and residues 1259-1270 represent additional C-terminal residues.

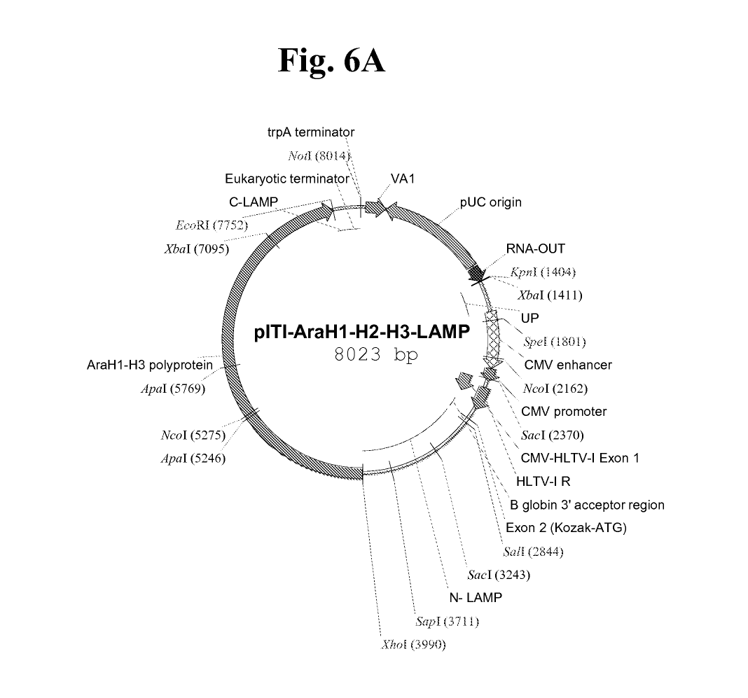

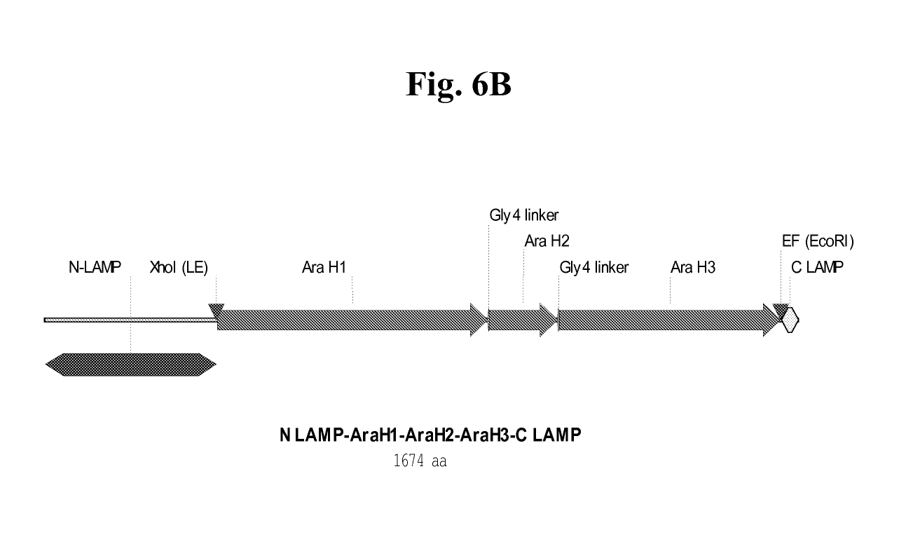

The nucleic acid constructs of the invention are essentially limitless in the number of allergens that can be coordinately produced. As such, two, three, four, five, six, ten, twenty, or more different allergens (from the same or a mixture of different sources) can be included in the nucleic acid constructs of the invention. FIG. 6A presents a vector map of another exemplary nucleic acid according to an embodiment of the invention. The vector or delivery vehicle includes a plasmid backbone with a pUC origin of replication and various transcription and expression elements for production of the encoded protein. The backbone can be, but is not necessarily, the pITI backbone of SEQ ID NO:1. The nucleic acid further comprises sequences for the encoded protein, which comprises an N-terminal region of the human LAMP protein, which includes a signal sequence domain and an intra-organelle stabilizing domain. The nucleic acid further provides sequences for an encoded chimeric protein that comprises the peanut allergen polyprotein AraH1/AraH2/AraH3. The nucleic acid further includes sequences encoding a portion of the C-terminal region of the human LAMP protein, which includes a transmembrane region and a targeting region. The nucleotide sequence for the coding region of the chimeric protein is provided as SEQ ID NO:8 The chimeric protein encoded by the vector of FIG. 6A is presented schematically in FIG. 6B (and as SEQ ID NO:9).

The domains present in the nucleic acids of the invention are described in more detail below with respect to the functions provided by the encoded chimeric proteins. It is to be understood that practice of the invention is not dependent upon or limited by any particular nucleic acid or protein sequence, but rather it is the combination of elements and domains that provides the advantages and properties to the constructs. It is also to be understood that the description relating to the various domains of the nucleic acid construct, when discussed in the context of the physical and functional characteristics of the encoded protein, and vice versa. It is sufficient to apprise one of skill in the art of the physical and functional characteristics of either the nucleic acids or the proteins. It is a simple matter using computers and the degeneracy of the genetic code to arrive at all possible nucleic acid molecules encoding known protein sequences and to arrive at proteins encoded by nucleic acids. Thus reference to a physical or functional characteristic of a particular protein sequence immediately discloses to the skilled artisan all of the possible nucleic acid sequences associated with that physical or functional characteristic, and vice versa.

It is also well within the skill of those of skill in the art to design and combine two or more nucleic acid molecules or sequences to arrive at a sequence encoding a chimeric protein according to the invention. Likewise, it is well within the skilled artisan's abilities to select and combine transcription and translation control elements to express the coding sequences and chimeric proteins in vivo or in vitro as desired. Accordingly, these commonly used techniques need not be discussed in detail herein to enable one to practice the present invention.

The nucleic acid of the invention comprises a signal sequence domain. The signal sequence domain contains a signal sequence that is provided for insertion of the encoded chimeric protein into a biological membrane that defines the border between an external environment and an internal environment. The signal sequence also directs transfer of the protein from the external environment to the internal environment. The general structure of a signal sequence is well known in the art, as are numerous examples of particular signal sequences. The practitioner is free to select any appropriate signal sequence according to the various selection parameters for each embodiment falling within the scope of this invention. In exemplary embodiments, the signal sequence is one that directs the chimeric protein to the endoplasmic reticulum. It is important to note at this juncture that the signal sequence domain is the only portion of the chimeric protein that contains a signal sequence. As such, the naturally-occurring signal sequences of allergens residing in the allergen domain have been removed prior to inclusion of the allergen sequences in the construct. It has been found that removal of these individual signal sequences improves the overall performance of the construct in vivo.

The nucleic acid of the invention comprises an intra-organelle stabilizing domain (IOSD). The IOSD comprises a sequence that encodes an amino acid sequence that binds, via chemical bonds, to one or more sequences in the allergen domain and protects those sequences from degradation (e.g., proteolysis) prior to arrival of the chimeric protein in the endosomal/lysosomal compartment. In essence, the IOSD can be envisioned as a protective cap for the allergen domain sequences, shielding those sequences, and in particular allergenic epitope sequences, from proteolytic enzymes, low pH, and other protein-destabilizing substances and conditions. The IOSD can be any of a number of known or engineered sequences, including, but not limited to, a LAMP polypeptide lumenal domain and the macrosialin/CD68 protein, which is a heavily glycosylated transmembrane protein that is expressed in macrophages and macrophage-like cells as a late endosomal protein. The key feature of the IOSD is the ability of the IOSD to bind to and protect the allergen domain from proteolysis until the MHC class II molecule is released from the invariant peptide. In this way, the three-dimensional structures of the allergenic epitope(s) are preserved until active MHC class II molecules are available for interaction. In preferred embodiments, the IOSD comprises all or part of the sequence of a lysosomal protein. In some embodiments, the IOSD is a protein or polypeptide other than a LAMP polypeptide lumenal domain, such as, but not limited to, macrosialin/CD68.

The nucleic acid construct of the invention comprises an allergen domain. The allergen domain comprises one or more sequences that encode allergen proteins, polypeptides, or peptides, which comprise one or more allergenic epitopes. The allergen domain does not include signal sequences from the allergens present. Numerous proteinaceous allergens are known in the art, and any one or combination of allergens and/or allergenic epitopes can be used in accordance with the present invention. Where less than a full-length allergenic sequence is used, preferably, one or more epitopes of the full-length allergen protein are provided in the context of their natural positions within the allergenic protein. More specifically, the present invention provides for improved nucleic acid vaccines, in which the vaccines encode chimeric proteins that retain or substantially retain their three dimensional structure until MHC class II molecules are competent to bind to epitopes on the chimeric proteins. In this way, an improved immune response can be elicited, as compared to delivery to the MHC class II molecules of short peptides, which generally will lack appropriate three dimensional structures. Accordingly, it is preferred that the allergen domain encode relatively long amino acid sequences that include one or more epitopes, if originally present on the allergen protein.

The allergen domain can include two or more allergens, each containing one or more allergenic epitopes. It is known that certain allergenic proteins contain two or more epitopes. As a preferred embodiment of the invention uses an entire allergenic coding region (i.e., the coding region lacking a signal sequence), or a substantial portion thereof, of an allergenic protein, certain allergen domains will include two or more epitopes in their naturally-occurring relationship. Alternatively, two or more known epitopes can be fused into one coding region. Yet again, in exemplary embodiments, two or more allergenic proteins, or allergenic regions thereof, are present in the allergen domain. Where two or more epitopes are engineered to be present in a single epitope domain, the epitopes can be from the same antigenic protein. Alternatively, they can be from two different proteins of the same species. Yet again, they can be from the same protein of two different species. Furthermore, they can be from two or more different proteins from two or more different species. In essence, any combination of epitopes from the same or different proteins from the same or different species is contemplated by this invention. Likewise, the order of the various allergens and epitopes can be varied in any way imaginable. The mixing of allergenic proteins and/or allergenic peptides from multiple species allows the creation of a robust nucleic acid vaccine that can provide treatment for allergies to a single source organism (e.g., particular species of tree) based on multiple allergens, as well as treatment for allergies to multiple source organisms (e.g., multiple plants that release spores during the same season of the year) based on multiple allergens. The ability to combat multiple allergies from a single nucleic acid vaccine has not been proven to date.

The nucleic acid construct of the invention further comprises a transmembrane domain. Transmembrane domains are well known and well characterized physical and functional elements of proteins that exist partially on both sides of a biological membrane. In essence, a transmembrane domain is a linear sequence of amino acids that are generally hydrophobic or lipophilic in nature and which function to anchor a protein at a biological membrane. Generally, such sequences are 20-25 residues in length. Those of skill in the art are well aware of such sequences and can easily obtain or engineer a suitable transmembrane sequence for use in the present invention.

In addition to the elements discussed above, the nucleic acid of the invention comprises a targeting domain. The targeting domain is a sequence that encodes an amino acid sequence that functions to target the encoded chimeric protein to the endosomal/lysosomal compartment. While not so limited in its identity, in preferred embodiments, the targeting domain comprises the C-terminal cytoplasmic targeting sequence of the LAMP polypeptide, DC-LAMP, LAMP2, LAMP-3, LIMP II, ENDOLYN, or macrosialin/CD68.

In embodiments, the nucleic acid of the invention comprises, as part of the allergen domain, the sequence of SEQ ID NO:2 (i.e., the Cry J2 nucleotide sequence lacking its signal sequence) or another sequence encoding SEQ ID NO:3 (i.e., the Cry J2 protein sequence lacking its signal sequence) in the allergen domain. SEQ ID NO:2 consists of nucleotides encoding the full protein coding sequence of Cry J2, with the exception of its signal sequence (i.e., SEQ ID NO:2), a pectate lysase protein found in the pollen of Cryptomeria japonica. Cry J2 is well known in the art to be correlated with seasonal and persistent allergies in areas where cedar pollen is present. IgE specific to Cry J2 is commonly found in allergic patients in areas near cedar groves. It is to be noted that, in the Sequence Listing provided as part of the disclosure of the invention, the signal sequence for each allergen, if present, is noted. It is to be understood that, within the context of the constructs of the invention, these signal sequences are not present.

In other embodiments, the nucleic acid comprises the sequence of SEQ ID NO:4 (i.e., the Cry J1 nucleotide sequence, lacking its signal sequence) or another sequence encoding SEQ ID NO:5 (i.e., the Cry J2 protein sequence lacking its signal sequence). In yet other embodiments, the nucleic acid comprises the sequence of both SEQ ID NO:2 and SEQ ID NO:4, or other sequences encoding SEQ ID NO:3 and SEQ ID NO:5, respectively. In embodiments, the nucleic acid comprises one or more of the other sequences disclosed herein, such as those encoding any of the following allergens: Cry J3 (Cry J3.8; C. japonica; SEQ ID NO:10; signal sequence is residues 1-26), CJP-4 (C. japonica; SEQ ID NO:11), CJP-6 (C. japonica; SEQ ID NO:12), CJP-8 (C. japonica; SEQ ID NO:13; signal sequence is residues 1-35), CPA63 (C. japonica; SEQ ID NO:14; signal sequence is residues 1-20), CJP38 (C. japonica; SEQ ID NO:15; signal sequence is residues 1-28), Cha o 1 (C. obtuse; SEQ ID NO:16; signal sequence is residues 1-21), Jun a 1 (J. ashei; SEQ ID NO:17; signal sequence is residues 1-21), Jun v 1 (J. virginiana: SEQ ID NO:18; signal sequence is residues 1-21), Cup a 1 (H. arizonica; SEQ ID NO:19; signal sequence is residues 1-21), Jun o 1 (J. oxycedrus; SEQ ID NO:20; signal sequence is residues 1-21), Cup s 1 (C. sempervirens; SEQ ID NO:21; signal sequence is residues 1-21) Cha o 2 (C. obtuse; SEQ ID NO:22; signal sequence is residues 1-22), Jun a 2 (J. ashei; SEQ ID NO:23; signal sequence is residues 1-22), Cup a 2 (H. arizonica; SEQ ID NO:24), Jun a 3 (J. ashei; SEQ ID NO:25; signal sequence is residues 1-16), Jun r 3 (J. rigida; SEQ ID NO:26; signal sequence is residues 1-26), Cup s 3 (C. sempervirens; SEQ ID NO:27; signal sequence is residues 1-26), Cup a 3 (H. arizonica; SEQ ID NO:28), Ch4A (P. monticola; SEQ ID NO:29; signal sequences is from residues 1-25), Ch4-1 (P. menziesii; SEQ ID NO:30; signal sequence is residues 1-26), PT-1 (P. taeda; SEQ ID NO:31), and LTP (P. abies; SEQ ID NO:32; signal sequence is from residues 1-25). Nucleic acid and amino acid sequences not listed with reference to SEQ ID NOs are also publicly available. It is a mere matter of computer program implementation to arrive at protein sequences according to the present invention based on the nucleic acid sequences. Of course, biochemically homologous sequences to these protein sequences are encompassed by these embodiments. For example, sequences showing 30% or more identity, such as 40% or more, 50% or more, 75% or more, 90% or more 95% or more, 98% or more, or 99% or more to the disclosed sequences are encompassed by these embodiments. It is to be understood that this concept applies not only to the particular sequences of allergens disclosed herein, but to all protein and nucleic acid sequences provided herein. Further, as stated above, each value within the disclosed ranges are understood to be specifically encompassed by the present disclosure.

In a particular instance of the invention, a DNA vaccine comprising SEQ ID NO:2 or another sequence encoding SEQ ID NO:3 within the allergen domain is provided. When such a vaccine is administered to a patient for whom there is considerable evidence of a Japanese red cedar allergy, the vaccine results in the de novo synthesis of a fusion or chimeric (these terms used interchangeably herein) protein comprising the allergen Cry J2 (presented within SEQ ID NO:3). Due to the combination of domains present on the chimeric protein, the protein is directed from the endoplasmic reticulum into the endolysosomal pathway, resulting in the processing of the fusion protein into epitopes in MHC vesicles, some of which become bound to MHC class II molecules, leading to an enhanced humoral immune response.

In another instance of the invention, a DNA vaccine comprising the sequence of SEQ ID NO:4 or another sequence encoding SEQ ID NO:5 within the allergen domain is provided. When such a vaccine is administered to a patient for whom there is considerable evidence of a Japanese red cedar allergy, the vaccine results in the de novo synthesis of a fusion or chimeric (these terms used interchangeably herein) protein comprising the allergen Cry J1 (found within the sequence of SEQ ID NO:4). Due to the combination of domains present on the chimeric protein, the protein is directed from the endoplasmic reticulum into the endolysosomal pathway, resulting in the processing of the fusion protein into epitopes in MHC vesicles, some of which become bound to MHC class II molecules, leading to an enhanced humoral immune response.

In another instance of the invention, a DNA vaccine comprising SEQ ID NO:6 within the allergen domain is provided. When such a vaccine is administered to a patient for whom there is considerable evidence of a Japanese red cedar allergy, the vaccine results in the de novo synthesis of a fusion or chimeric (these terms used interchangeably herein) protein comprising the allergens CryJ1 and Cry J2 (SEQ ID NO:7). Due to the combination of domains present on the chimeric protein, the protein is directed from the endoplasmic reticulum into the endolysosomal pathway, resulting in the processing of the fusion protein into epitopes in MHC vesicles, some of which become bound to MHC class II molecules, leading to an enhanced humoral immune response.

In another instance of the invention, a nucleic acid encoding the full protein coding sequence of Jun a1, a pectate lysase belonging to the genus Juniperus ashei, is provided in the allergen domain. Jun a1 demonstrates a high degree of sequence identity with Cry J1 and both retains a similar enzymatic activity to Cry J1 and possesses a high similarity in known epitopes.

Other polypeptides are well known to be cross-reactive to Cry J1 and that this cross-reactivity is due to shared epitopes related to the enzymatic activity of pectate lysase family polypeptides. The family includes the major allergen of Japanese cypress (Chamaecyparis obtusa (Ch ol)), and includes allergens from: Juniperus ashei (Jun a 1), Juniperus virginiana (Jun v 1), Cuppressus arizonica (Cup a 1), Juniperus oxycedrus (Jun o 1), and Cupressus sempervirens (Cup s 1). It has been observed in the literature that there is strong cross-reactivity among allergic patients to pollen from the cedar family (Cupressus). Table I, below, depicts a table showing levels of cross-reactivity among related proteins. While the invention is described in detail with regard to Cry J1 and Cry J2, it is to be understood that one or more of the allergens disclosed herein and particularly in Table I can be used in addition to or as alternatives to the Cry J1 and Cry J2 sequences.

TABLE I
Cryptomeria japonica cross-reactivity to other allergens

| Cryptomeria | Cha | Jun | Jun | Cup | Jun | Cup | Cha | Jun | Cup | Jun | Jun | Cup | Cup |
|---|---|---|---|---|---|---|---|---|---|---|---|---|---|
| Japonica | o 1 | a 1 | v 1 | a 1 | o 1 | s 1 | o 2 | a 2 | a 2 | a 3 | r 3 | s 3 | a 3 |
| Cry J1 | 80% | 79% | 80% | 75% | 85% | 85% | | | | | | | |
| Cry J2 | | | | | | | 74% | 71% | 80% | | | | |
| Cry J3 | | | | | | | | | | 86% | 87% | 85% | 87% |

| Cryptomeria | | | | |
|---|---|---|---|---|
| Japonica | Ch4A | Ch4-1 | PT-1 | LTP |
| CJP-4 | 70% | 57% | | |
| CJP-6 | | | 74% | |
| CJP-8 | | | | 45% |
| CPA63 | | | | |

It is well known in the art that certain sites for inserting a nucleotide sequence for a coding region into the nucleotide sequence for a different coding region (i.e., fusion sites) are more favorable than others. The location of the allergen sequence taught in, for example, FIGS. 1-5 is taught as the favorable location for using the composition taught in the present invention. It is within the scope of the present invention to move the location of the allergen sequence to other locations, such as within the luminal domain of a LAMP polypeptide or other intra-organelle stabilizing domain. However, it is preferred that the allergen is not placed within the coding region of either the transmembrane or cytoplasmic domain. In a preferred instance of the invention, the allergen sequence is inserted into the luminal domain of a LAMP polypeptide within 5 amino acids from the junction with the transmembrane domain and up to 20 amino acids on the 5′ N terminal side of a LAMP polypeptide luminal domain.

The nucleic acid of the invention can be provided as a purified or isolated molecule. The nucleic acid also can be provided as part of a composition. The composition can consist essentially of the nucleic acid, meaning that the nucleic acid is the only nucleic acid in the composition suitable for expression of a coding sequence. Alternatively, the composition can comprise a nucleic acid of the invention. In exemplary embodiments, the composition is a pharmaceutical composition comprising the nucleic acid of the invention along with one or more pharmaceutically acceptable substances or carriers. In some embodiments, the composition comprises a substance that promotes uptake of the nucleic acid by a cell. In some embodiments, the composition comprises a targeting molecule that assists in delivering the nucleic acid to a specific cell type, such as an immune cell (e.g., and APC). In embodiments, the nucleic acid is part of a delivery vehicle or delivery vector for delivery of the nucleic acid to a cell or tissue.

In a particular instance of the invention, the composition comprises a mixture of two DNA vaccines, where one vaccine comprises the sequence of one allergen and where the other vaccine comprises the sequence of another allergen. The two vaccine constructs can be mixed together at a ratio of 1:1, 1:2, 1:3, 1:4, sequentially up to 1:10. The preferred ratio is 1:1.

In a particular instance of the invention, the nucleic acids of Cry J1, Cry J2, and/or Jun a2 are present within a nucleic acid delivery vector. In a preferred embodiment of the invention, the nucleic acid delivery vector does not contain an antibiotic resistance gene, such as the nucleic acid delivery vector taught by U.S. patent application publication number 2008/006554, or vectors that are disclosed in or result from U.S. patent application publication number 2006/003148. In a particular instance of the invention, the nucleic acid is a viral vector, such as an adenoviral vector.

The nucleic acids and compositions are novel and useful as agents for reducing allergic reactions in patients. For example, the nucleic acids and compositions are useful in reducing pollinosis in patients with a demonstrated allergic reaction correlated with Japanese red cedar pollen, or from a homologous pollen or allergen. As another non-limiting example, the nucleic acids are useful for reducing food allergies, such as allergy to peanuts or other nuts. Delivery of nucleic acids and compositions to treat pollinosis from Japanese red cedar pollen, such that the nucleic acids and compositions transfect an antigen presenting cell, results in an increase in serum levels of immunoglobulin G (IgG) specific to epitopes contained within Cry J1 and/or Cry J2. This response is useful for the reduction of allergy symptoms. Delivery of allergens for other allergies, including ragweed, other tree pollens, and foods also result in increase in serum levels of IgG.

Methods of treating subjects in need are also provided by this invention. The methods are methods of prophylactically treating or therapeutically treating a subject suffering from or at risk of developing an allergic reaction to one or more allergens. The method comprises administering to the subject a DNA vaccine according to the invention in an amount sufficient to cause uptake of and expression of the DNA vaccine by an APC. Expression of the DNA vaccine results in presentation of the encoded allergenic epitope(s) on the APC, and development of an IgG immune response.

In a particular instance of the invention, SEQ ID NO:2, SEQ ID NO:4, and/or another allergen encoding sequence are administered to a cell. In preferred embodiments, the cell is an antigen presenting cell, such as a dendritic cell. Preferably, the dendritic cell is a human dendritic cell. The present invention can be administered by methods known in the art to be effective delivery methods for nucleic acid vaccines, including intramuscular injection, subcutaneous injection, electroporation, gene gun vaccination, or liposome-mediated transfer.

This invention provides a formulation useful for the treatment of pollinosis correlated with Japanese red cedar pollen. It has previously been determined that delivering a DNA plasmid encoding the protein coding sequence of an allergen to an animal can increase IFN-gamma production and lower IL-4 production, which is useful in treating animals allergic to the specific allergen. The present invention provides an improved DNA vaccine composition for treating patients with an allergy correlated to Japanese red cedar pollen. The fusion protein of the invention has a specific intracellular trafficking pattern that intersects with MHC class II vesicles, and results in enhanced presentation of allergen epitopes to the immune system, specifically resulting in an enhanced antibody response. Nucleic acids and compositions provided by the present invention are useful for conducting allergy immunotherapy.

The present invention provides a formulation that when administered to a cell results in an increased specific antibody response. The increased antibody response to the allergen is useful for treating an IgE-mediated allergic disease. IgE has certain properties related to its cellular restriction and the resulting intracellular signaling upon binding cognate allergen. IgE is generated against an allergen when B cells receive IL-4 secreted by Th2 cells. This helps instruct B cells to produce IgE class antibodies. Upon secretion by B cells, IgE binds to Fc-eRI, its high affinity receptor expressed by mast cells and eosinophils, resulting in these cells and the animal becoming sensitized to future allergen exposure. Consequently, the symptoms of allergy can be triggered upon the ingestion, inhalation, or mucosal contact with an allergen. Due to the binding properties of antibodies, it has been proposed that one way of reducing allergy symptoms is to chelate free allergen available for binding by IgE through competition with other antibody classes. In particular, an allergy formulation that increases IgG has been proposed to be an pathway for reducing allergic disease. The invention described herein induces enhanced IgG production, thus causing a decrease in the ratio of IgE to IgG in a clinically significant manner. The results of studies that have been conducted indicate that at day 98, the level of IgG induced by a Cry J2-LAMP construct is greater than that induced by delivery of nucleotides encoding unmodified Cry J2.

In another instance of the invention, a method is taught for selecting pectate lysase polypeptides found in the pollen of a cedar tree, for determining the degree of sequence homology with the amino acid or nucleic acid sequence of a Cry J1, a pectate lysase, so that a new composition of matter similar to Cry J1 can be generated, and so that administration of the homologous composition of matter to a patient would produce a therapeutic result useful for treating allergies correlated with cedar pollen.

### EXAMPLES

The invention will now be described with reference to exemplary embodiments of the invention. The following examples are intended to give the reader a better understanding of the construction and activity of the constructs of the invention, and should not be construed as a limitation on the scope of the invention.

### Example 1

### General Materials and Methods

Immunizations and Sera Collection

Six to eight week old female BALB/c mice were purchased from Harlan Laboratories, Frederick, Md. and maintained at our animal facility in Rockville, Md. The DNA immunizations were given either intramuscularly or intradermal with 50 ug of plasmid DNA in a volume of 100 ul of sterile PBS. Sera were obtained by orbital bleed and stored at −20° C. for later analysis. For sensitization, mice were injected with either 5 ug/ml of recombinant CRYJ2 (rCRYJ2) or recombinant CRYJ1 (rCYRJ1) together with 100 ul of alum (2 mg/ml) in a total volume of 200 ul. Mice were bled weekly and sera were analyzed for CRYJ specific antibodies by ELISA.

Guinea Pigs

Female Guinea pigs were purchased and housed at Spring Valley Laboratories (Woodline, Md.). The DNA immunizations were given intramuscularly with 100 ug of plasmid DNA in a volume of 200 ul of sterile saline. Sera were obtained by cardiac bleed and store at 20° C. for later analysis.

Detection of CYRJ2-Specific Immunoglobulin Responses

Nunc Maxisorp immunoassay plates were coated with rCRYJ2 at a concentration of 5 ug/ml in PBS overnight at 4° C. After blocking with 1% BSA in PBS, sera were diluted in PBS containing 0.05% Tween-20 (PBS-T) added and incubated for 1 hour. The IgG, IgG1, or IgG2a bound to the CRYJ2 immobilized on the wells was detected using peroxidase conjugated goat anti-mouse IgG, IgG1 or IgG2a antibodies (Jackson Laboratories). TMB substrate (KPL) was added and the enzymatic activity stopped with TMB Stop Solution. The plates were read at 450 nm. In some instances, Sure Stop Solution (KPL) was used and plates were read at 650 nm.

Preparation of Splenocytes for Cytokine Measurements

Spleens were removed aseptically and teased to prepare a single-cell suspension. To study the primary response, splenocytes were cultured in 24-well plates (4×10⁵ cells/well) in the presence or absence of 10 ug/ml, 5 ug/ml, or 2.5 ug/ml of rCRYJ2 for 72 hours.

Cytokine Assays

Supernatants were assayed for the presence of IFN-gamma and IL-4 by ELISA. Matched antibody pairs were used for IFN-gamma and IL-4 and done according to manufacturer's instructions. The standard curves were generated with mouse recombinant IFN-gamma and IL-4. All antibodies and cytokines were purchased from Invitrogen, Carlsbad, Calif. The detection limits of IFN-gamma and IL-4 assays were 20 and 10 pg/ml in respective.

### Example 2

### Expression of Allergens from Constructs

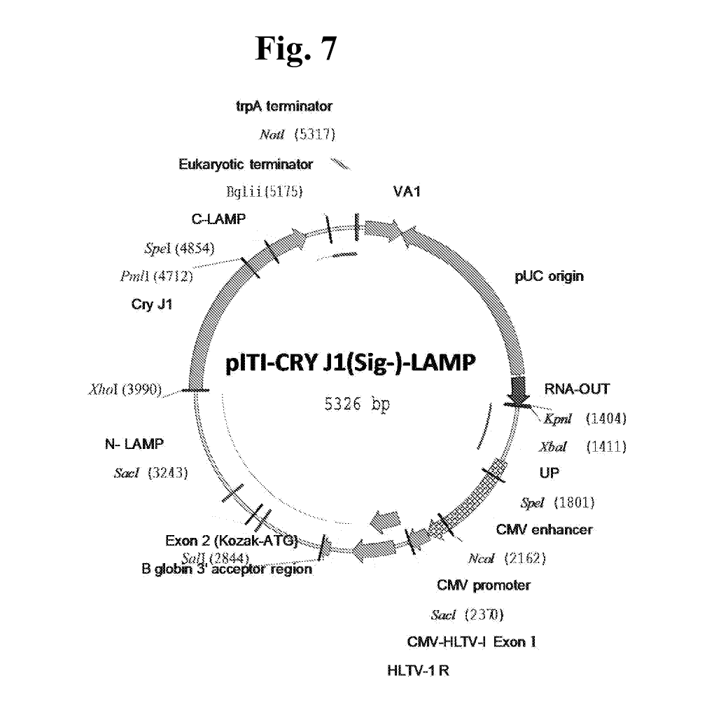

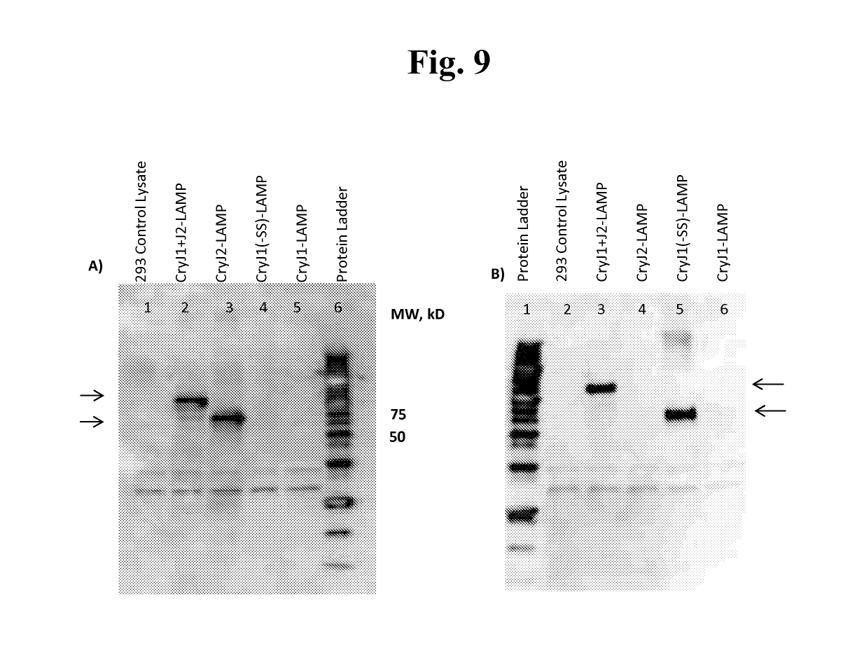

To show that the nucleic acid constructs of the invention can be used to express one or multiple allergens in transformed cells, human 293 cells were transfected with the CryJ2-LAMP plasmid, CryJ1+J2-LAMP plasmid (FIG. 4), CryJ1-LAMP plasmid, CryJ1 plasmid (lacking the CryJ1 signal sequence; FIG. 7), and the base plasmid vector alone (negative control; SEQ ID NO:1). The results of the experiments are shown in FIG. 9.

FIG. 9A shows the results of the transfection reactions, with detection using an anti-CryJ2 antibody. Briefly, thirty micrograms of cell lysate was electrophoresed, then transferred to a membrane for immunoblotting. Proteins were detected by immunoblotting with a CryJ2 monoclonal antibody, followed by chemiluminescence. As can be seen from the Figure, constructs comprising the CryJ2 allergen alone, and the CryJ1+CryJ2 allergens were detected (lanes 2 and 3), whereas other allergens were not. In this experiment, the naturally-occurring signal sequences for the CryJ1 and CryJ2 allergens were removed prior to the experiment, except for the construct in lane 5. These results show not only that the constructs of the invention are suitable for expression of allergens, but also that multiple allergens can be co-expressed.

FIG. 9B shows the results of the transfection reactions, with detection using an anti-CryJ1 antibody. Briefly, thirty micrograms of cell lysate was electrophoresed, then transferred to a membrane for immunoblotting. Proteins were detected by immunoblotting with a CryJ1 monoclonal antibody, followed by chemiluminescence. As can be seen from the Figure, constructs comprising the CryJ1+CryJ2 allergens (lacking natural signal sequences) were detected (lane 3), as was the construct comprising the CryJ1 allergen in which the naturally-occurring signal sequence had been removed (lane 5). However, the construct in which the Cry1 allergen, which included its natural signal sequence, was not detected. These results show that the constructs of the invention are suitable for expression and detection of multiple allergens, and that removal of naturally-occurring signal sequences is important in expressing and detecting products.

### Example 3

### Data Supporting MHC II Processing Pathway for Constructs

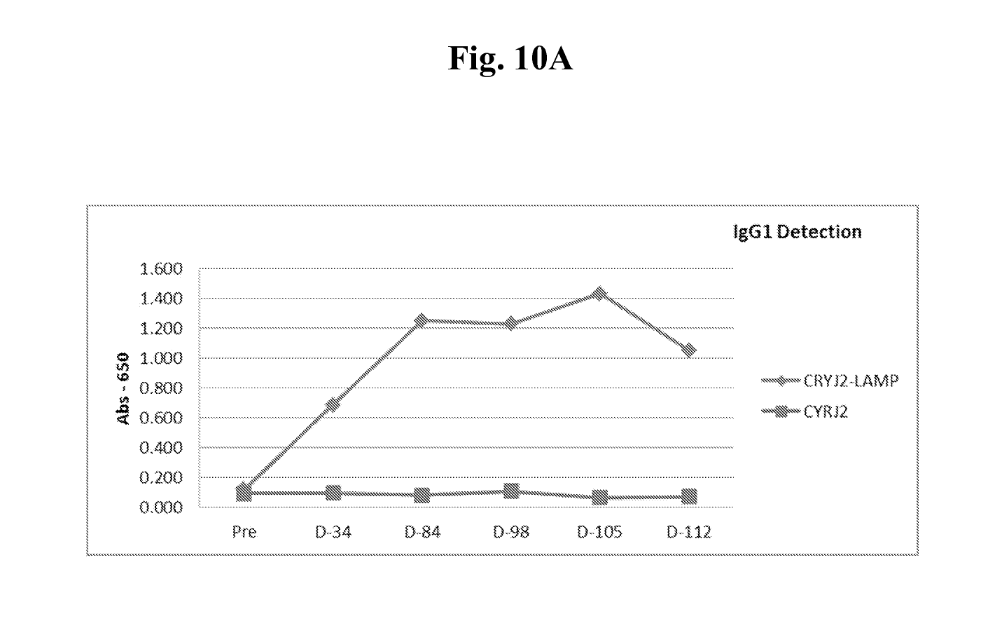

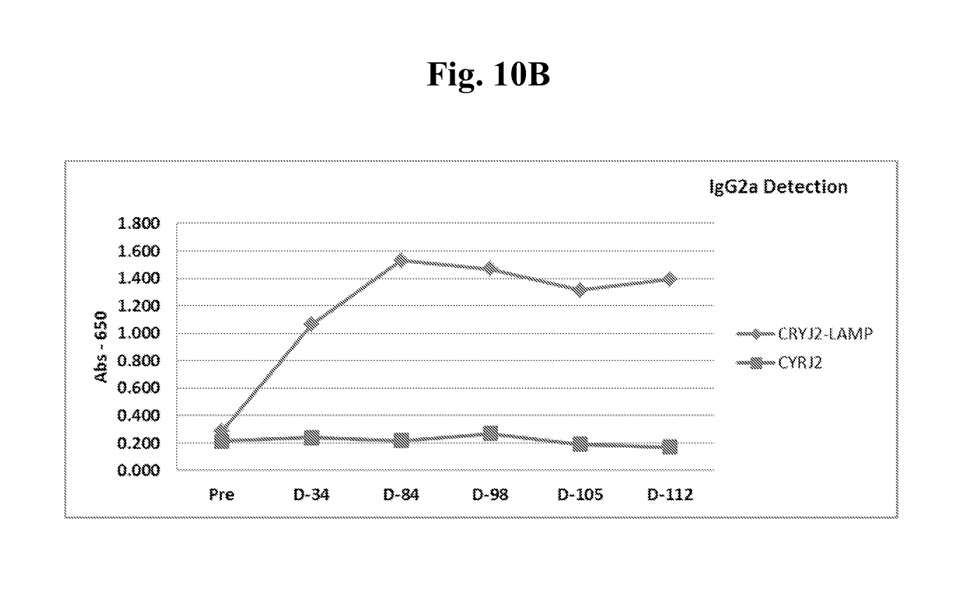

To determine if chimeric proteins produced from the constructs of the invention are processed through the MHC II pathway, a set of experiments was performed to compare the immune response to the CryJ2 protein when administered as a coding region on a plasmid or as an allergen domain on a construct according to the present invention. The results are presented in FIG. 10, Panels A and B.

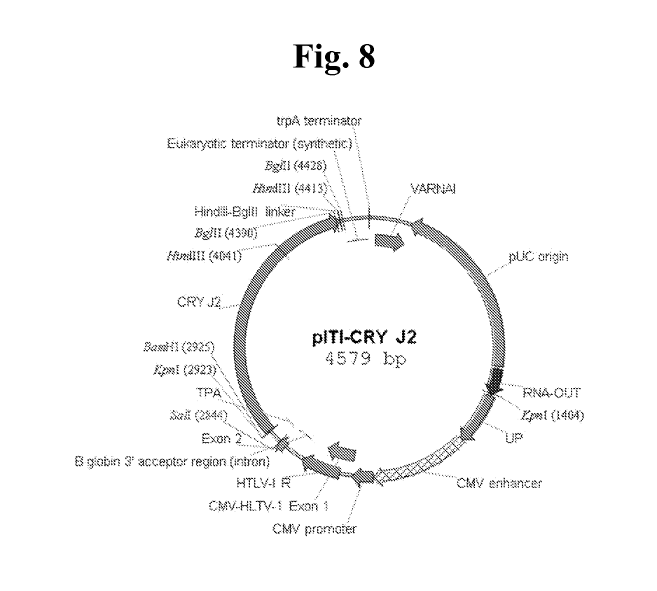

More specifically, the figure shows the CryJ2 specific response following four DNA immunizations and crude pollen extract sensitization. Groups of mice (n=5) were immunized subcutaneously with either CRYJ2-LAMP plasmid DNA or CRYJ2 plasmid (see FIG. 8) DNA on days 0, 7, 14, and 21. Six weeks (day 77) following the last DNA immunization, the mice were sensitized with crude pollen extract in alum and given a booster three weeks (day 91) later. The data show the values generated from the pooled sera for each time point. IgG1 (Panel A) and IgG2a (Panel B) response in mice receiving CRYJ2-LAMP DNA remained elevated through day 112 and well above those mice that received CRYJ2 plasmid DNA that did not include LAMP. Delivery of allergens by way of constructs according to the invention thus provide a superior MHC II response than delivery of allergens without the context of the constructs of the invention.

### Example 4

### Dosing Rationales

### Comparison of Immune Response to Different Doses of Constructs and to Vector Alone

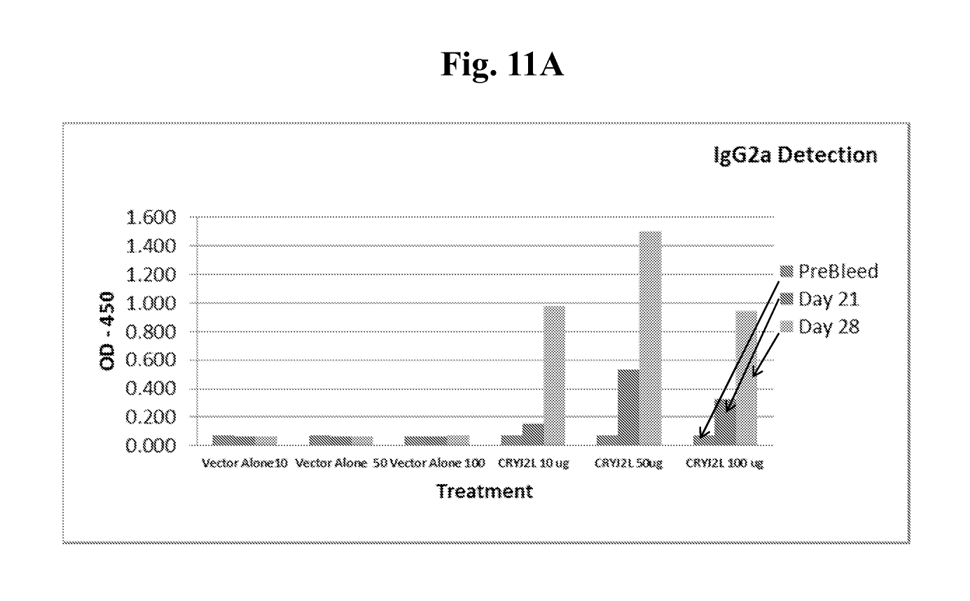

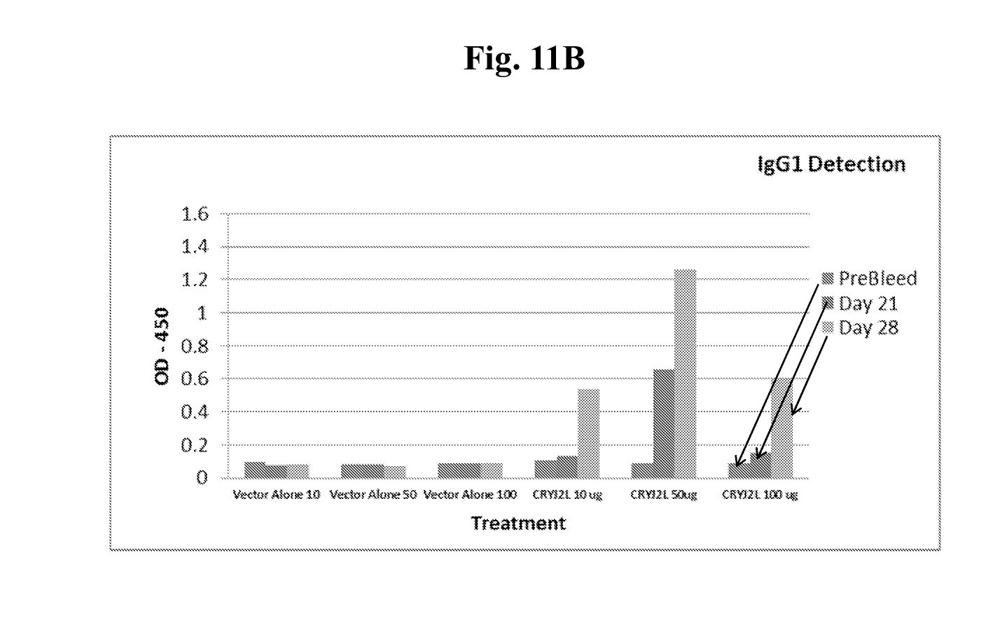

FIG. 11 shows a CryJ2-specific response following four DNA immunizations at different levels of dosing, for both IgG2a production and IgG1 production. Groups of mice (n=5) were given either 10 ug, 50 ug, or 100 ug of CRYJ2-LAMP plasmid DNA or Vector DNA intramuscularly on days 0, 7, 14, and 21. Three weeks following the last DNA immunization, the mice were sacrificed and spleens removed for Cytokine Induction assays.

The data show the values generated from the pooled sera for each vaccine dose. All three concentrations of CRYJ2-LAMP plasmid DNA gave IgG1 and IgG2a responses, with the 50 ug dose appearing to have given the highest antibody response. Vector alone, at any of the concentrations, did not induce any antibody response. These data show that there is a dose-dependent response for invoking an immune response, and that the immune response is, at least in part, an MHC II type response.

### Example 5

### Further Data Showing an Immune Response Via the MHC II Pathway

In this set of experiments, secretion of cytokines in supernatants of stimulated spleen cells was determined using IL-4 and IFN-gamma as markers. Specifically, spleen cells of mice (n=3) were harvested at day 42 and cultured in the presence of 10 ug/ml, 5 ug/ml, 2.5 ug/ml, or no rCRYJ2. Spleen cells from naïve mice were used as a negative control. IL-4 and IFN-gamma levels of rCRYJ2 stimulated splenocytes were measured by ELISA in pg/ml.

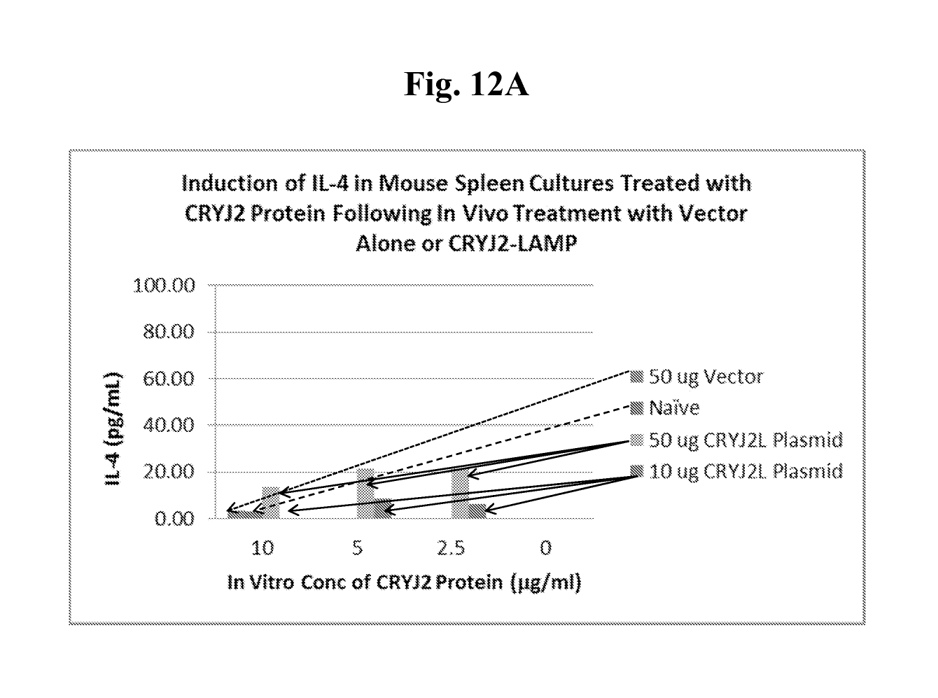

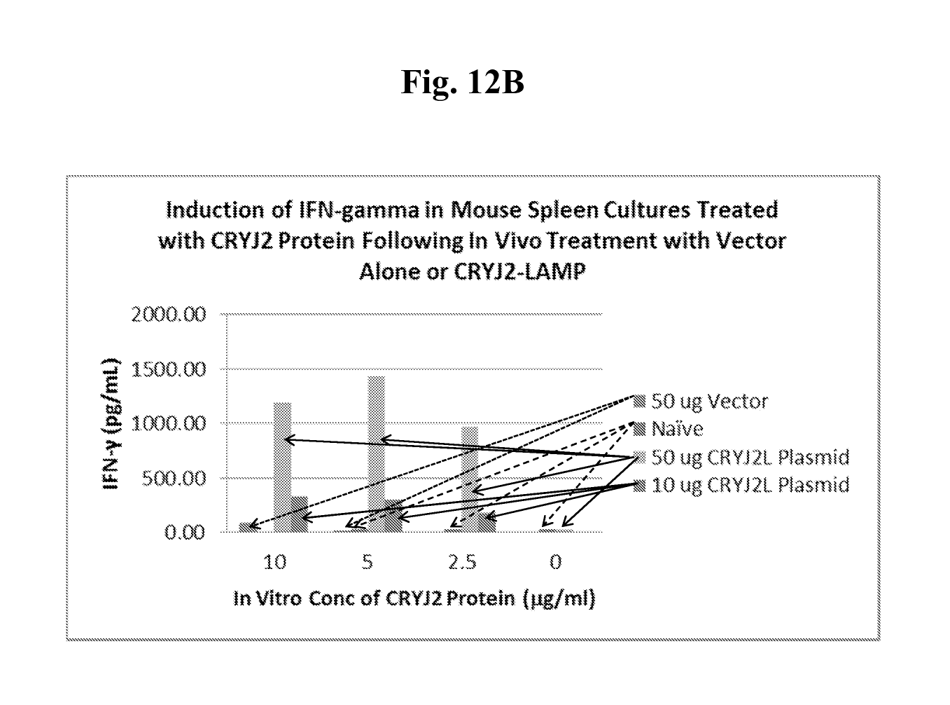

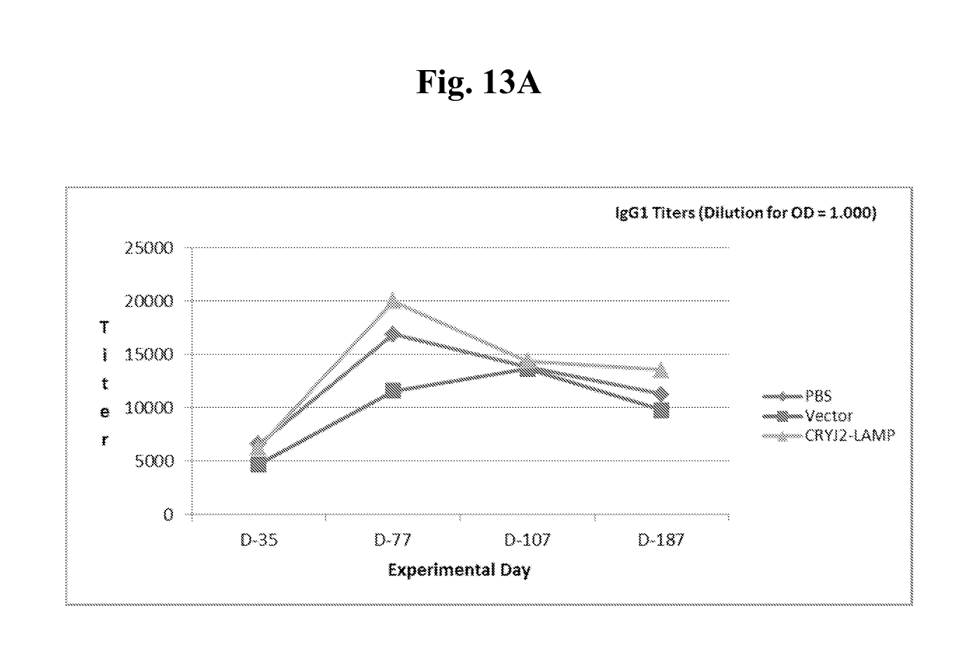

The data are presented in FIG. 12, Panels A and B. The data show that mice receiving 50 ug of CRYJ2-LAMP plasmid DNA had a significantly higher expression of IFN-gamma (an established biomarker for activation of the MHC II immune response pathway) than those receiving the lower dose of plasmid DNA. There was very little response seen of IL-4 levels in any of the groups, an established biomarker for the MHC I pathway. There was also very little response, if any, with IL-5 (data not shown). These results indicate that Cry J2-LAMP DNA immunization induced the recruitment of Th1 memory cells and not Th2 cells, as indicated by the production of IFN-gamma and not IL-4 after stimulation with the recombinant Cry J2 protein.

### Example 6

### Studies on the Therapeutic Effect of Immunization with CryJ2-LAMP DNA Vaccine in Previously CryJ2 Sensitized Mice

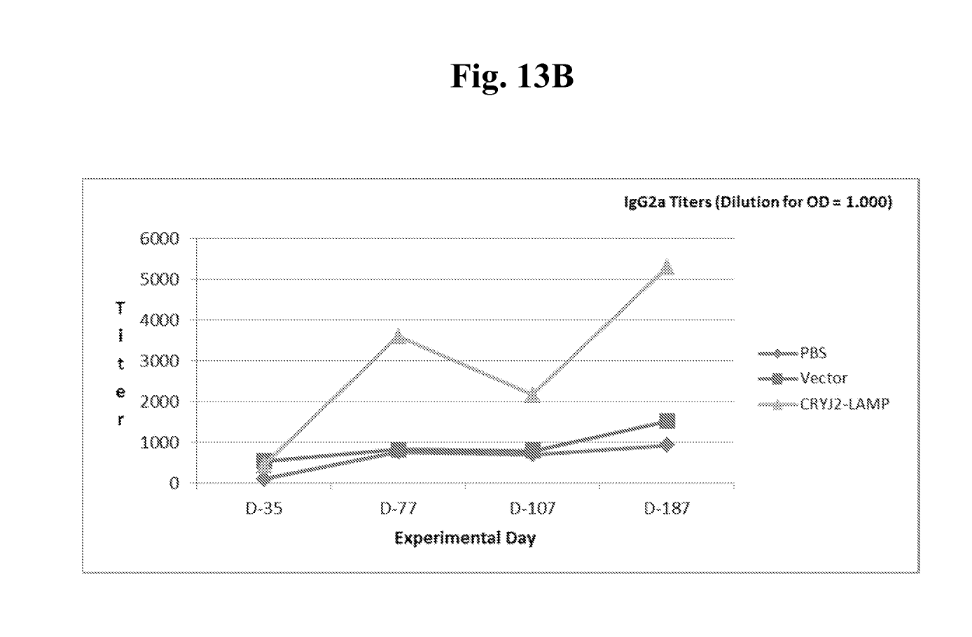

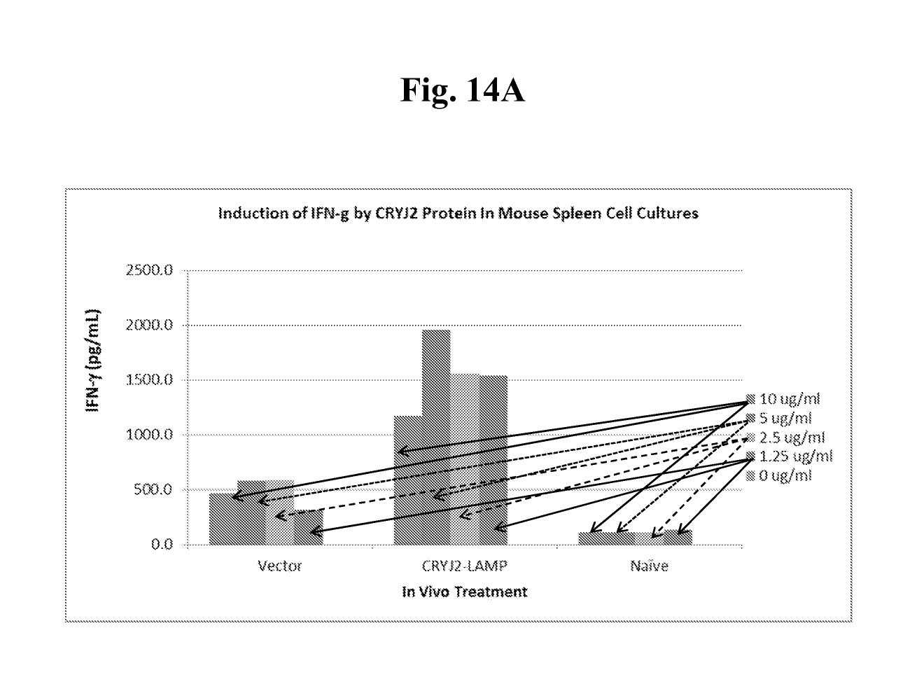

To study the therapeutic effects of the DNA-LAMP-CryJ2 vaccine, groups of mice (n=5) were sensitized with three injections of 5 ug of rCRYJ2 recombinant protein and four weeks later, treated with four injections of CRYJ2-LAMP plasmid DNA given in a weekly (7 day) intervals. The DNA immunizations induced a booster effect for IgG2a and a transient increase for IgG1 antibodies indicating a Th1-directed modulatory effect of the DNA vaccine. Two additional DNA immunizations on days 167 and 174 boosted the CRYJ2 specific IgG2a response and almost no change in IgG1 response. Visual examination of the mice revealed no physical distress or skin reactions. There were also no changes in appetite nor did they appear lethargic. The effects on IgG1 and IgG2a titers are shown in FIG. 13, Panels A and B, respectively.

### Example 7

### Induction of IFN-Gamma and IL-4 in Mouse Spleen Cell Cultures

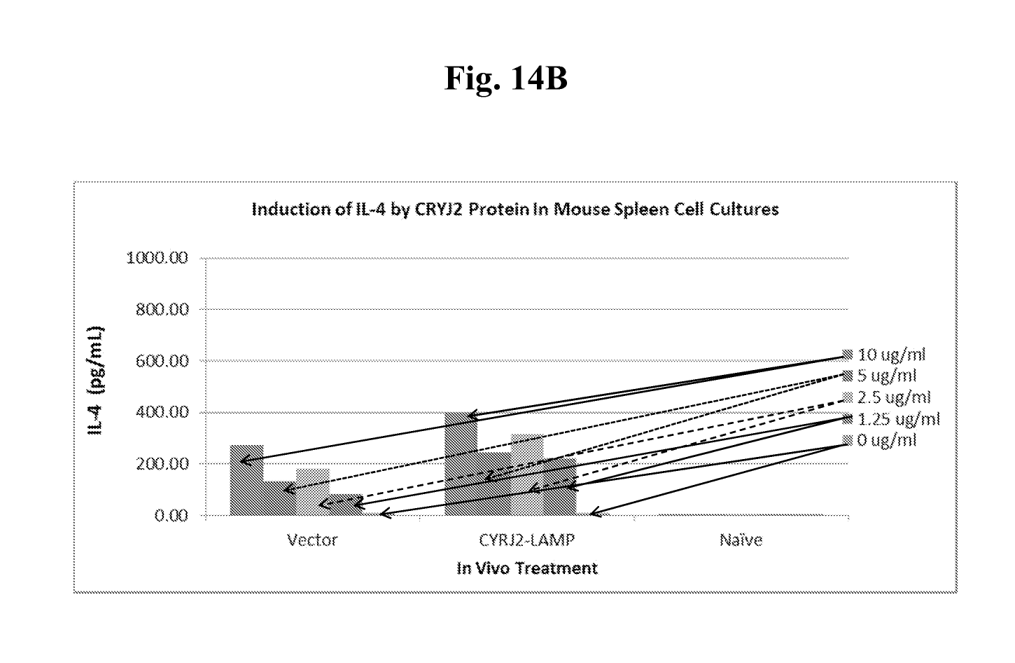

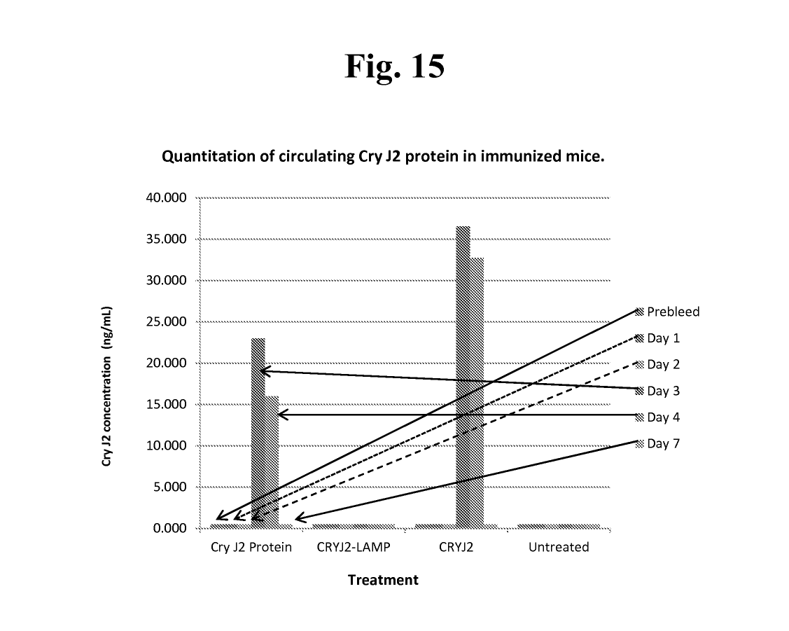

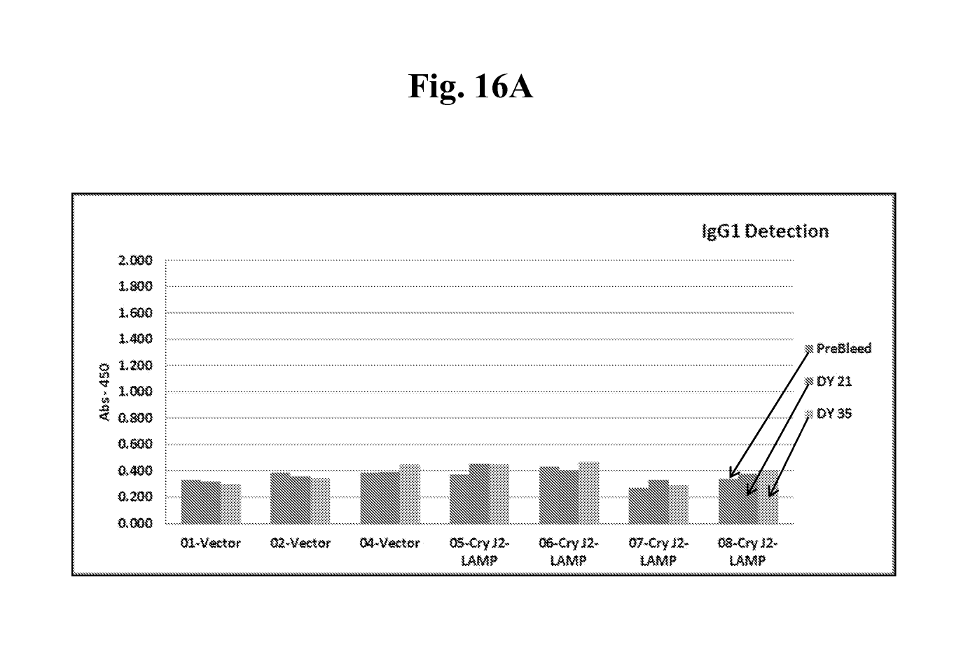

The therapeutic effect of CryJ2-LAMP DNA vaccine was also studied in terms of cytokine induction. Spleen cells of mice (n=3) were harvested at day 183 and stimulated with varying concentrations of rCRYJ2. Spleen cells from naïve mice were used as a negative control. IL-4 and IFN-gamma levels of rCRYJ2 stimulated splenocytes were measured by ELISA in pg/ml. Significantly elevated expression of IFN-gamma was detected in the CRYJ2-LAMP vaccinated group compared with that in the vector group. However, IL-4 expression showed no difference from the vector group. The increase in IFN-gamma as a result of Cry J2-LAMP DNA immunization presumably involves the recruitment of antigen-specific Th1 cells and the creation of a Th1 cytokine milieu. Data obtained from this experiment is presented in FIG. 14, Panels A and B.

### Example 8

### Detection of Circulating CryJ2 Protein in Sera

Mice were immunized with Cry J2 protein, pDNA-Cry J2 (no LAMP) and Cry J2-LAMP-vax. Serum samples were taken at days 0, 1, 2, 3, 4, and 7 and evaluated for the presence of free Cry J2 protein in a sensitive sandwich immunoassay. Free Cry J2 was detected in the protein and non-LAMP immunization. However, no free allergen was detected in any time point in any experiment with Cry J2-LAMP-vax immunized mice (minimum detectable level 2 ng/ml). Data supporting these statements are provided in FIG. 12.

LAMP vaccines according to the invention will be the only formulations that treat allergies without introducing free allergen into the patient systemically. This is unlike traditional immunotherapy which can sometimes result in anaphylactic reactions due to systemic introduction of allergen. This experiment shows that mice which received the Cry J2-LAMP DNA plasmid did not have free Cry J2 protein and thus not released into the systemic circulation as seen with mice given protein alone or Cry J2 DNA without LAMP.

### Example 9

### Effectiveness of DNA Vaccines in Guinea Pigs

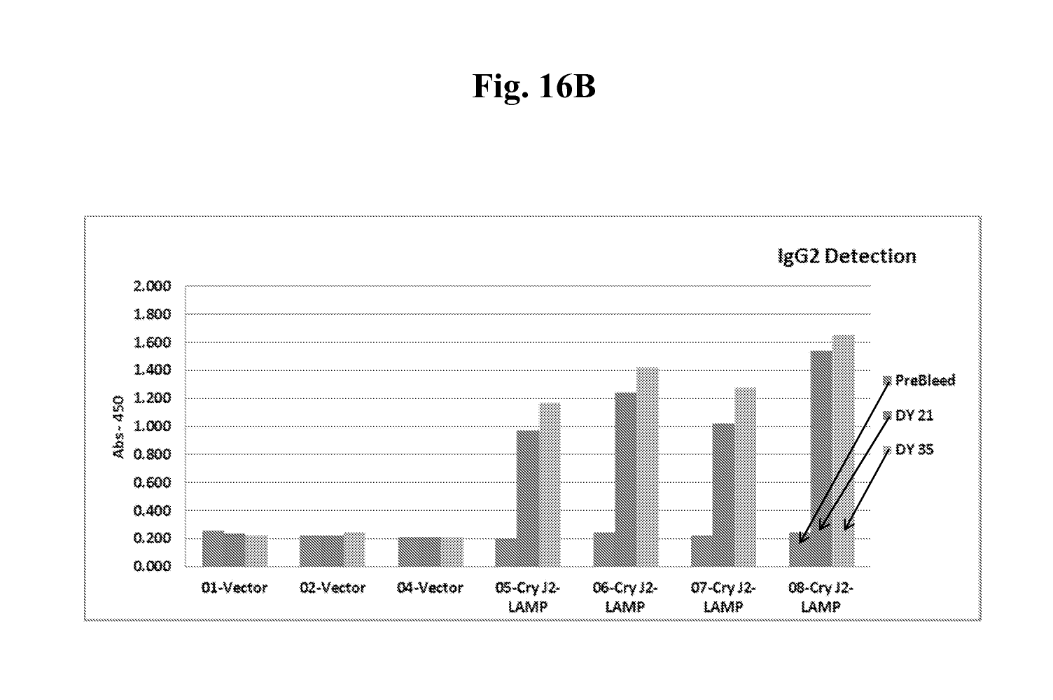

To expand the scientific understanding of the function of the present nucleic acid constructs in other mammals, studies were performed in female guinea pigs immunized with the CryJ2-LAMP DNA vaccine, then challenged with recombinant CryJ2 protein. The results of the studies are shown in FIG. 16, Panels A and B.

Specifically, female guinea pigs received intramuscular injections of 100 ug of CRYJ2-LAMP DNA Vaccine or vector alone on days 0, 7, and 14. Four weeks following the last DNA vaccine immunization on day 14, the guinea pigs received subcutaneous injections of 10 ug/ml of rCRYJ2 protein/alum on days 42 and 49. Serum samples were obtained from guinea pigs on days 0, 21, 35, 63, and 77. The data show that the mean absorbance values for the guinea pigs receiving CRYJ2-LAMP DNA increased through day 35 for IgG2 with little or no IgG1 response. The increase in IgG2a is consistent with what is typically seen in a Th1 biased response.

### Example 10

### Further Investigation in Other Mammals

### Toxicology Data Showing Safety

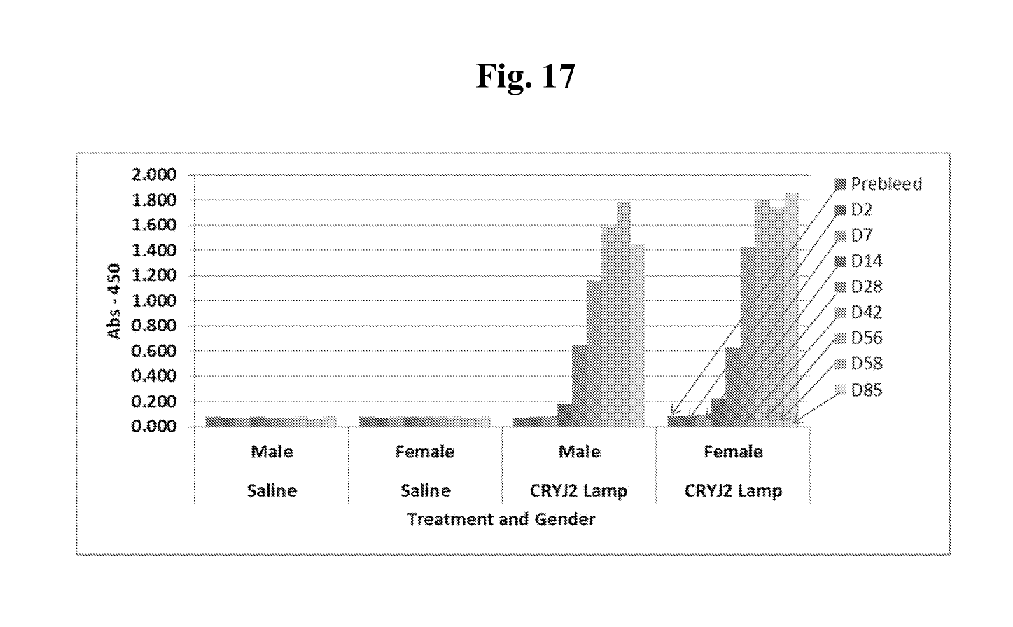

New Zealand White rabbits received intramuscular injections of 4.128 mg of CRYJ2-LAMP DNA. Age and gender-matched control rabbits received saline alone. Rabbits were immunized on days 1, 14, 28, 42, and 56. Serum samples were obtained from rabbits on days 1, 14, 28, 42, 56, 58, and 85. Mean absorbance values of rabbit serum at 1:100 following multiple IM injections of CryJ2-LAMP plasmid or saline are shown in FIG. 17. As can be seen from the Figure, the data show that the mean absorbance values for the rabbits receiving saline are less than 0.100. The absorbance values of rabbits in the groups treated with CRYJ2-LAMP DNA generally increased through day 42 and in some cases increased through day 85.

### Example 11

### Applicability to Food Allergies

Over the last 25 years, 8 significant peanut allergens have been identified based on sensitization in peanut allergic patients. Three major peanut allergens are most commonly recognized by IgE of peanut allergic individuals: 65-100% recognize Ara h1, a 63.5 kDa seed storage vicilin family protein; 71-100% recognize Ara h2, a 17 kDa seed storage conglutin family protein; and 45-95% recognize Ara h3, a 14 kDa seed storage glycinin family protein. In addition to being a common causative agent in triggering peanut-dependent allergic reactions and anaphylaxis, these three proteins also appear to promote stronger allergic reactions. Targeting these allergens as the basis for peanut allergy immunotherapy has the potential of providing the broadest protection from strong allergic reactions among the diverse population of peanut allergics. Phase I clinical trials are currently underway that use hypoallergenic forms of the three major allergens and a heat killed bacterium adjuvant as allergy immunotherapy. This trial is ongoing, but the eventual commercialization of such a therapy will be a challenge due to a highly complex manufacturing process.

To address the rising incidence of food allergies, and in particular peanut allergy, a nucleic acid construct according to the invention was created. The construct is depicted in FIG. 6A, and a schematic of the encoded chimeric protein is depicted in FIG. 6B, as discussed above. This construct can be used to generate a predominantly MHC II response in subjects to which it is administered. The presence of the three most common peanut allergens in a single chimeric protein allows for a broad immunization, which will treat the vast majority of peanut allergies in the population.

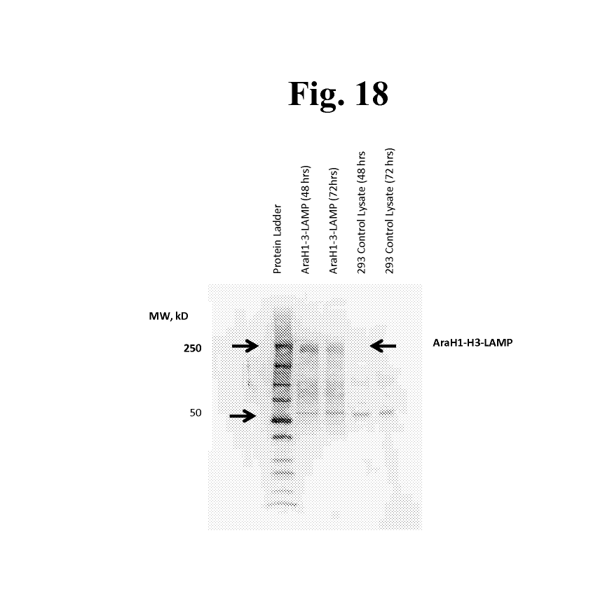

The construct was expressed and the results shown in FIG. 18. FIG. 19 shows that all three allergens can be expressed and detected as a single poly-protein on Western blots.

It will be apparent to those skilled in the art that various modifications and variations can be made in the practice of the present invention and in construction of the nucleic acid constructs without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

### Further Sequences for Sequence Listing

In addition to the sequences provided in the formal Sequence Listing provided as part of this application, the following sequences comprise part of the present disclosure:
1. The nucleotide sequence of the coding region for the Cry1-Cry2-LAMP chimeric construct, as follows:

| Cry J1 + J2-LAMP | |
|---|---|
| SEQ ID NO: 6 | |
| ccgcctaatg agcgggcttt tttttcttag ggtgcaaaag gagagcctgt aagcgggcac | 60 |
| tcttccgtgg tctggtggat aaattcgcaa gggtatcatg gcggacgacc ggggttcgag | 120 |
| ccccgtatcc ggccgtccgc cgtgatccat gcggttaccg cccgcgtgtc gaacccaggt | 180 |
| gtgcgacgtc agacaacggg ggagtgctcc ttttggcttc cttccccttc ttccgcttcc | 240 |
| tcgctcactg actcgctgcg ctcggtcgtt cggctgcggc gagcggtatc agctcactca | 300 |
| aaggcggtaa tacggttatc cacagaatca ggggataacg caggaaagaa catgtgagca | 360 |
| aaaggccagc aaaaggccag gaaccgtaaa aaggccgcgt tgctggcgtt tttccatagg | 420 |
| ctccgccccc ctgacgagca tcacaaaaat cgacgctcaa gtcagaggtg gcgaaacccg | 480 |
| acaggactat aaagatacca ggcgtttccc cctggaagct ccctcgtgcg ctctcctgtt | 540 |
| ccgaccctgc cgcttaccgg atacctgtcc gcctttctcc cttcgggaag cgtggcgctt | 600 |
| tctcatagct cacgctgtag gtatctcagt tcggtgtagg tcgttcgctc caagctgggc | 660 |
| tgtgtgcacg aaccccccgt tcagcccgac cgctgcgcct tatccggtaa ctatcgtctt | 720 |
| gagtccaacc cggtaagaca cgacttatcg ccactggcag cagccactgg taacaggatt | 780 |
| agcagagcga ggtatgtagg cggtgctaca gagttcttga agtggtggcc taactacggc | 840 |
| tacactagaa gaacagtatt tggtatctgc gctctgctga agccagttac cttcggaaaa | 900 |
| agagttggta gctcttgatc cggcaaacaa accaccgctg gtagcggtgg tttttttgtt | 960 |
| tgcaagcagc agattacgcg cagaaaaaaa ggatctcaag aagatccttt gatcttttct | 1020 |
| acggggtctg acgctcagtg gaacgaaaac tcacgttaag ggattttggt catgagatta | 1080 |
| tcaaaaagga tcttcaccta gatcctttta aattaaaaat gaagttttaa atcaatctaa | 1140 |
| agtatatatg agtaaacttg gtctgacagt taccaatgct taatcagtga ggcacctatc | 1200 |
| tcagcgatct gtctatttcg ttcatccata gttgcctgac tcctgcaaac cacgttgtgg | 1260 |
| tagaattggt aaagagagtc gtgtaaaata tcgagttcgc acatcttgtt gtctgattat | 1320 |
| tgatttttgg cgaaaccatt tgatcatatg acaagatgtg tatctacctt aacttaatga | 1380 |
| ttttgataaa aatcattagg taccccggct ctagatggca tgacattaac ctataaaaat | 1440 |
| aggcgtatca cgaggccctt tcgtctcgcg cgtttcggtg atgacggtga aaacctctga | 1500 |
| cacatgcagc tcccggagac ggtcacagct tgtctgtaag cggatgccgg gagcagacaa | 1560 |
| gcccgtcagg gcgcgtcagc gggtgttggc gggtgtcggg gctggcttaa ctatgcggca | 1620 |
| tcagagcaga ttgtactgag agtgcaccat atgcggtgtg aaataccgca cagatgcgta | 1680 |
| aggagaaaat accgcatcag attggctatt ggccattgca tacgttgtat ccatatcata | 1740 |
| atatgtacat ttatattggc tcatgtccaa cattaccgcc atgttgacat tgattattga | 1800 |
| ctagttatta atagtaatca attacggggt cattagttca tagcccatat atggagttcc | 1860 |
| gcgttacata acttacggta aatggcccgc ctggctgacc gcccaacgac ccccgcccat | 1920 |
| tgacgtcaat aatgacgtat gttcccatag taacgccaat agggactttc cattgacgtc | 1980 |
| aatgggtgga gtatttacgg taaactgccc acttggcagt acatcaagtg tatcatatgc | 2040 |
| caagtacgcc ccctattgac gtcaatgacg gtaaatggcc cgcctggcat tatgcccagt | 2100 |
| acatgacctt atgggacttt cctacttggc agtacatcta cgtattagtc atcgctatta | 2160 |
| ccatggtgat gcggttttgg cagtacatca atgggcgtgg atagcggttt gactcacggg | 2220 |
| gatttccaag tctccacccc attgacgtca atgggagttt gttttggcac caaaatcaac | 2280 |
| gggactttcc aaaatgtcgt aacaactccg ccccattgac gcaaatgggc ggtaggcgtg | 2340 |
| tacggtggga ggtctatata agcagagctc gtttagtgaa ccgtcagatc gcctggagac | 2400 |
| gccatccacg ctgttttgac ctccatagaa gacaccggga ccgatccagc ctccgcggct | 2460 |
| cgcatctctc cttcacgcgc ccgccgccct acctgaggcc gccatccacg ccggttgagt | 2520 |
| cgcgttctgc cgcctcccgc ctgtggtgcc tcctgaactg cgtccgccgt ctaggtaagt | 2580 |
| ttaaagctca ggtcgagacc gggcctttgt ccggcgctcc cttggagcct acctagactc | 2640 |
| agccggctct ccacgctttg cctgaccctg cttgctcaac tctagttctc tcgttaactt | 2700 |
| aatgagacag atagaaactg gtcttgtaga aacagagtag tcgcctgctt ttctgccagg | 2760 |
| tgctgacttc tctcccctgg gcttttttct ttttctcagg ttgaaaagaa gaagacgaag | 2820 |
| aagacgaaga agacaaaccg tcgtcgacat ggcgccccgc agcgcccggc gacccctgct | 2880 |
| gctgctactg ctgttgctgc tgctcggcct catgcattgt gcgtcagcag caatgtttat | 2940 |
| ggtgaaaaat ggcaacggga ccgcgtgcat aatggccaac ttctctgctg ccttctcagt | 3000 |
| gaactacgac accaagagtg gccctaagaa catgaccctt gacctgccat cagatgccac | 3060 |
| agtggtgctc aaccgcagct cctgtggaaa agagaacact tctgacccca gtctcgtgat | 3120 |
| tgcttttgga agaggacata cactcactct caatttcacg agaaatgcaa cacgttacag | 3180 |
| cgtccagctc atgagttttg tttataactt gtcagacaca caccttttcc ccaatgcgag | 3240 |
| ctccaaagaa atcaagactg tggaatctat aactgacatc agggcagata tagataaaaa | 3300 |
| atacagatgt gttagtggca cccaggtcca catgaacaac gtgaccgtaa cgctccatga | 3360 |
| tgccaccatc caggcgtacc tttccaacag cagcttcagc cggggagaga cacgctgtga | 3420 |
| acaagacagg ccttccccaa ccacagcgcc ccctgcgcca cccagcccct cgccctcacc | 3480 |
| cgtgcccaag agcccctctg tggacaagta caacgtgagc ggcaccaacg ggacctgcct | 3540 |
| gctggccagc atggggctgc agctgaacct cacctatgag aggaaggaca acacgacggt | 3600 |
| gacaaggctt ctcaacatca accccaacaa gacctcggcc agcgggagct gcggcgccca | 3660 |
| cctggtgact ctggagctgc acagcgaggg caccaccgtc ctgctcttcc agttcgggat | 3720 |
| gaatgcaagt tctagccggt ttttcctaca aggaatccag ttgaatacaa ttcttcctga | 3780 |
| cgccagagac cctgccttta aagctgccaa cggctccctg cgagcgctgc aggccacagt | 3840 |
| cggcaattcc tacaagtgca acgcggagga gcacgtccgt gtcacgaagg cgttttcagt | 3900 |
| caatatattc aaagtgtggg tccaggcttt caaggtggaa ggtggccagt ttggctctgt | 3960 |
| ggaggagtgt ctgctggacg agaacagcct cgaggacaat cctattgatt cctgctggcg | 4020 |
| tggagattct aactgggcac agaaccggat gaaactggct gactgtgccg tgggctttgg | 4080 |
| ctcttccact atgggaggga agggaggcga cctgtacact gttacaaaca gcgacgacga | 4140 |
| ccctgtcaat ccagcacccg gaaccttgag atatggtgca acgcgagacc gaccactttg | 4200 |
| gatcatcttt agcggaaaca tgaacatcaa gttgaagatg cctatgtaca tagctgggta | 4260 |
| caaaaccttc gacggcagag gagcccaagt gtacattggc aacggaggtc cctgcgtgtt | 4320 |
| catcaagcgt gttagtaatg tgatcattca cggtctgcac ctctatggct gttcaacaag | 4380 |
| cgtgctgggg aatgtgctga tcaatgagtc attcggtgtt gaacccgtgc acccacagga | 4440 |
| cggtgatgcg ttgacactga ggacagccac caatatctgg attgaccata acagtttctc | 4500 |
| taacagctca gatggcctgg tggatgtcac cttgagtagc acaggggtca caatcagcaa | 4560 |
| caatctgttc ttcaaccatc ataaggtgat gctgctgggc cacgacgatg cgtattccga | 4620 |
| cgataagagc atgaaagtga cggtggcctt taaccagttt ggtcctaact gtggacagcg | 4680 |
| gatgcctaga gccaggtacg gactggtgca cgtggccaac aacaactatg atccgtggac | 4740 |
| tatctatgca attggcggtt cttccaaccc gacgatactg agtgaaggga actcctttac | 4800 |
| cgctcccaat gagagctaca agaagcaggt caccatccgc ataggctgca aaactagttc | 4860 |
| atcctgtagc aactgggtgt ggcagtccac tcaagatgtc ttctacaacg gagcttactt | 4920 |
| cgttagcagt gggaaatacg aaggtggcaa catatacaca aagaaagagg ctttcaatgt | 4980 |
| ggagaatggc aatgccactc cccagctcac caagaatgca ggggtgctca cctgctccct | 5040 |
| gagcaaacgg tgcggcggtg gtggcctcga ggatcagtca gcgcagatca tgctggatag | 5100 |
| cgtggtggag aagtacctga ggagtaacag gtcactgcgc aaggttgagc attccagaca | 5160 |
| cgacgctatc aacatcttca acgtggagaa gtacggtgct gtcggagacg ggaagcacga | 5220 |
| ctgcaccgaa gccttttcta cagcctggca agctgcctgc aagaatccct cagccatgct | 5280 |
| cctcgtgcct gggtctaaga agtttgtcgt gaataacctt ttcttcaatg gaccctgcca | 5340 |
| gccacacttt accttcaaag ttgatgggat catcgcagcc tatcagaacc cagctagctg | 5400 |
| gaagaacaat cggatctggt tgcagtttgc caaactgaca ggattcaccc tgatggggaa | 5460 |
| aggcgtgatc gacggacagg gcaaacagtg gtgggcaggg cagtgcaagt gggtcaatgg | 5520 |
| tagggagatt tgcaatgaca gggaccgtcc taccgctatc aagtttgatt tcagcacagg | 5580 |
| actgattatt caggggttga agctgatgaa tagtccagag tttcaccttg tgtttggcaa | 5640 |
| ttgtgaaggt gtgaagatca taggcattag cattacagca cctcgcgatt ctcccaatac | 5700 |
| ggacggcatt gacatcttcg cctccaagaa ctttcacctg caaaagaata ccattggcac | 5760 |
| aggcgacgac tgcgtggcca ttggcactgg cagcagcaat atcgttatcg aagatttgat | 5820 |
| atgtggtcct gggcatggca taagcattgg aagcctgggt agagaaaact caagagctga | 5880 |
| agtcagctat gttcacgtta acggagcgaa gttcattgat acccagaacg gactgcgaat | 5940 |
| caaaacttgg caagggggaa gtggcatggc atctcacatc atctacgaga acgtcgagat | 6000 |
| gatcaattcc gagaacccca tactgattaa ccaattctat tgtacttccg cctctgcctg | 6060 |
| ccagaatcag agatcagccg tgcagattca ggacgtgaca tacaagaata tccgagggac | 6120 |
| gagcgctacc gctgccgcaa tacagctcaa atgttccgat agcatgccct gcaaagatat | 6180 |
| caagcttagt gatatctccc tcaaactgac tagcggaaag atagcgtcct gtctcaatga | 6240 |
| taacgcaaat ggctacttct cagggcatgt gatccctgca tgcaaaaacc ttagcccgag | 6300 |
| tgcgaaacgc aaagaatcca aatcccataa gcatccgaag actgtgatgg tcgagaacat | 6360 |
| gagagcctac gacaaaggga accggacgag gattctgctg ggctctcgac cgccaaactg | 6420 |
| taccaacaaa tgtcacggtt gttctccatg caaagctaaa ctggtgatag tgcatcgcat | 6480 |
| catgcctcaa gagtactatc cccagcgttg gatttgtagt tgccatggca agatctatca | 6540 |
| cccagaattc acgctgatcc ccatcgctgt gggtggtgcc ctggcggggc tggtcctcat | 6600 |
| cgtcctcatc gcctacctcg tcggcaggaa gaggagtcac gcaggctacc agactatcta | 6660 |
| gtaaggatct ttttccctct gccaaaaatt atggggacat catgaagccc cttgagcatc | 6720 |
| tgacttctgg ctaataaagg aaatttattt tcattgcaat agtgtgttgg aattttttgt | 6780 |
| gtctctcact cggaaggaca taagggcggc cgctagc | 6817 |

2. The nucleic acid sequence of the coding region for the Ara H1/H2/h3 polyprotein:

| SEQ ID NO: 8 | |
|---|---|
| ccgcctaatg agcgggcttt tttttcttag ggtgcaaaag gagagcctgt aagcgggcac | 60 |
| tcttccgtgg tctggtggat aaattcgcaa gggtatcatg gcggacgacc ggggttcgag | 120 |
| ccccgtatcc ggccgtccgc cgtgatccat gcggttaccg cccgcgtgtc gaacccaggt | 180 |
| gtgcgacgtc agacaacggg ggagtgctcc ttttggcttc cttccccttc ttccgcttcc | 240 |
| tcgctcactg actcgctgcg ctcggtcgtt cggctgcggc gagcggtatc agctcactca | 300 |
| aaggcggtaa tacggttatc cacagaatca ggggataacg caggaaagaa catgtgagca | 360 |
| aaaggccagc aaaaggccag gaaccgtaaa aaggccgcgt tgctggcgtt tttccatagg | 420 |
| ctccgccccc ctgacgagca tcacaaaaat cgacgctcaa gtcagaggtg gcgaaacccg | 480 |
| acaggactat aaagatacca ggcgtttccc cctggaagct ccctcgtgcg ctctcctgtt | 540 |
| ccgaccctgc cgcttaccgg atacctgtcc gcctttctcc cttcgggaag cgtggcgctt | 600 |
| tctcatagct cacgctgtag gtatctcagt tcggtgtagg tcgttcgctc caagctgggc | 660 |
| tgtgtgcacg aaccccccgt tcagcccgac cgctgcgcct tatccggtaa ctatcgtctt | 720 |
| gagtccaacc cggtaagaca cgacttatcg ccactggcag cagccactgg taacaggatt | 780 |
| agcagagcga ggtatgtagg cggtgctaca gagttcttga agtggtggcc taactacggc | 840 |
| tacactagaa gaacagtatt tggtatctgc gctctgctga agccagttac cttcggaaaa | 900 |
| agagttggta gctcttgatc cggcaaacaa accaccgctg gtagcggtgg tttttttgtt | 960 |
| tgcaagcagc agattacgcg cagaaaaaaa ggatctcaag aagatccttt gatcttttct | 1020 |
| acggggtctg acgctcagtg gaacgaaaac tcacgttaag ggattttggt catgagatta | 1080 |
| tcaaaaagga tcttcaccta gatcctttta aattaaaaat gaagttttaa atcaatctaa | 1140 |
| agtatatatg agtaaacttg gtctgacagt taccaatgct taatcagtga ggcacctatc | 1200 |
| tcagcgatct gtctatttcg ttcatccata gttgcctgac tcctgcaaac cacgttgtgg | 1260 |
| tagaattggt aaagagagtc gtgtaaaata tcgagttcgc acatcttgtt gtctgattat | 1320 |
| tgatttttgg cgaaaccatt tgatcatatg acaagatgtg tatctacctt aacttaatga | 1380 |
| ttttgataaa aatcattagg taccccggct ctagatggca tgacattaac ctataaaaat | 1440 |
| aggcgtatca cgaggccctt tcgtctcgcg cgtttcggtg atgacggtga aaacctctga | 1500 |
| cacatgcagc tcccggagac ggtcacagct tgtctgtaag cggatgccgg gagcagacaa | 1560 |
| gcccgtcagg gcgcgtcagc gggtgttggc gggtgtcggg gctggcttaa ctatgcggca | 1620 |
| tcagagcaga ttgtactgag agtgcaccat atgcggtgtg aaataccgca cagatgcgta | 1680 |
| aggagaaaat accgcatcag attggctatt ggccattgca tacgttgtat ccatatcata | 1740 |
| atatgtacat ttatattggc tcatgtccaa cattaccgcc atgttgacat tgattattga | 1800 |
| ctagttatta atagtaatca attacggggt cattagttca tagcccatat atggagttcc | 1860 |
| gcgttacata acttacggta aatggcccgc ctggctgacc gcccaacgac ccccgcccat | 1920 |
| tgacgtcaat aatgacgtat gttcccatag taacgccaat agggactttc cattgacgtc | 1980 |
| aatgggtgga gtatttacgg taaactgccc acttggcagt acatcaagtg tatcatatgc | 2040 |
| caagtacgcc ccctattgac gtcaatgacg gtaaatggcc cgcctggcat tatgcccagt | 2100 |
| acatgacctt atgggacttt cctacttggc agtacatcta cgtattagtc atcgctatta | 2160 |
| ccatggtgat gcggttttgg cagtacatca atgggcgtgg atagcggttt gactcacggg | 2220 |
| gatttccaag tctccacccc attgacgtca atgggagttt gttttggcac caaaatcaac | 2280 |
| gggactttcc aaaatgtcgt aacaactccg ccccattgac gcaaatgggc ggtaggcgtg | 2340 |
| tacggtggga ggtctatata agcagagctc gtttagtgaa ccgtcagatc gcctggagac | 2400 |
| gccatccacg ctgttttgac ctccatagaa gacaccggga ccgatccagc ctccgcggct | 2460 |
| cgcatctctc cttcacgcgc ccgccgccct acctgaggcc gccatccacg ccggttgagt | 2520 |
| cgcgttctgc cgcctcccgc ctgtggtgcc tcctgaactg cgtccgccgt ctaggtaagt | 2580 |
| ttaaagctca ggtcgagacc gggcctttgt ccggcgctcc cttggagcct acctagactc | 2640 |
| agccggctct ccacgctttg cctgaccctg cttgctcaac tctagttctc tcgttaactt | 2700 |
| aatgagacag atagaaactg gtcttgtaga aacagagtag tcgcctgctt ttctgccagg | 2760 |
| tgctgacttc tctcccctgg gcttttttct ttttctcagg ttgaaaagaa gaagacgaag | 2820 |
| aagacgaaga agacaaaccg tcgtcgacat ggcgccccgc agcgcccggc gacccctgct | 2880 |
| gctgctactg ctgttgctgc tgctcggcct catgcattgt gcgtcagcag caatgtttat | 2940 |
| ggtgaaaaat ggcaacggga ccgcgtgcat aatggccaac ttctctgctg ccttctcagt | 3000 |
| gaactacgac accaagagtg gccctaagaa catgaccctt gacctgccat cagatgccac | 3060 |
| agtggtgctc aaccgcagct cctgtggaaa agagaacact tctgacccca gtctcgtgat | 3120 |
| tgcttttgga agaggacata cactcactct caatttcacg agaaatgcaa cacgttacag | 3180 |
| cgttcagctc atgagttttg tttataactt gtcagacaca caccttttcc ccaatgcgag | 3240 |
| ctccaaagaa atcaagactg tggaatctat aactgacatc agggcagata tagataaaaa | 3300 |
| atacagatgt gttagtggca cccaggtcca catgaacaac gtgaccgtaa cgctccatga | 3360 |
| tgccaccatc caggcgtacc tttccaacag cagcttcagc aggggagaga cacgctgtga | 3420 |
| acaagacagg ccttccccaa ccacagcgcc ccctgcgcca cccagcccct cgccctcacc | 3480 |
| cgtgcccaag agcccctctg tggacaagta caacgtgagc ggcaccaacg ggacctgcct | 3540 |
| gctggccagc atggggctgc agctgaacct cacctatgag aggaaggaca acacgacggt | 3600 |
| gacaaggctt ctcaacatca accccaacaa gacctcggcc agcgggagct gcggcgccca | 3660 |
| cctggtgact ctggagctgc acagcgaggg caccaccgtc ctgctcttcc agttcgggat | 3720 |
| gaatgcaagt tctagccggt ttttcctaca aggaatccag ttgaatacaa ttcttcctga | 3780 |
| cgccagagac cctgccttta aagctgccaa cggctccctg cgagcgctgc aggccacagt | 3840 |
| cggcaattcc tacaagtgca acgcggagga gcacgtccgt gtcacgaagg cgttttcagt | 3900 |
| caatatattc aaagtgtggg tccaggcttt caaggtggaa ggtggccagt ttggctctgt | 3960 |
| ggaggagtgt ctgctggacg agaacagcct cgagaagtcc agcccctacc agaagaaaac | 4020 |
| cgagaacccc tgcgcccagc ggtgcctgca gtcttgtcag caggaacccg acgacctgaa | 4080 |
| gcagaaggcc tgcgagagcc ggtgcaccaa gctggaatac gaccccagat gcgtgtacga | 4140 |
| ccctagaggc cacaccggca ccaccaacca gagaagccct ccaggcgagc ggaccagagg | 4200 |
| cagacagcct ggcgactacg acgacgacag acggcagccc agaagagaag agggcggcag | 4260 |
| atggggacct gccggcccta gagagagaga acgcgaggaa gattggagac agcccagaga | 4320 |
| ggactggcgg aggccttctc accagcagcc ccggaagatc agacccgagg gcagagaagg | 4380 |
| cgagcaggaa tggggcacac ctggctctca cgtgcgcgag gaaaccagcc ggaacaaccc | 4440 |
| cttctacttc ccctcccggc ggttcagcac cagatacggc aaccagaacg gccggatcag | 4500 |
| agtgctgcag agattcgacc agcggagccg gcagttccag aacctgcaga accaccggat | 4560 |
| cgtgcagatc gaggccaagc ccaacaccct ggtgctgccc aaacacgccg acgccgacaa | 4620 |
| catcctcgtg atccagcagg gccaggccac cgtgacagtg gccaacggca acaacagaaa | 4680 |
| gagcttcaac ctggacgagg gccacgccct gagaatcccc agcggcttca tcagctacat | 4740 |
| cctgaacaga cacgacaatc agaacctgag ggtggccaag atcagcatgc ccgtgaacac | 4800 |
| ccctggccag ttcgaggact tcttccccgc atcctcccgg gaccagagca gctacctgca | 4860 |
| gggcttcagc cggaataccc tggaagccgc cttcaacgcc gagttcaacg agatcagacg | 4920 |
| ggtgctgctg gaagagaacg ctggcggaga gcaggaagaa cggggccaga gaagatggtc | 4980 |
| caccagaagc agcgagaaca acgagggcgt gatcgtgaag gtgtccaaag aacacgtgga | 5040 |
| agaactgacc aagcacgcca agagcgtgtc caagaagggc tccgaggaag agggggacat | 5100 |
| caccaacccc atcaatctga gagagggcga gcccgacctg agcaacaact tcggcaagct | 5160 |
| gttcgaagtg aagcccgaca agaagaaccc ccagctgcag gacctggaca tgatgctgac | 5220 |
| ctgcgtggaa atcaaagagg gggccctgat gctgccacac ttcaactcca aagccatggt | 5280 |
| catcgtggtc gtgaacaagg gcaccggcaa cctggaactg gtggccgtgc ggaaagagca | 5340 |
| gcagcagaga ggccgcagag aggaagaaga ggacgaggac gaagaagaag agggatccaa | 5400 |
| ccgggaagtg cggcggtaca ccgccagact gaaagaaggc gacgtgttca tcatgcctgc | 5460 |
| cgcccacccc gtggccatca atgcctctag cgagctgcat ctgctgggct tcggcattaa | 5520 |
| cgccgagaac aatcaccgga tctttctggc cggcgacaaa gacaacgtga tcgaccagat | 5580 |
| cgagaagcag gccaaggacc tggcctttcc cggctctggc gaacaagtgg aaaagctgat | 5640 |
| caagaaccag aaagaaagcc acttcgtgtc cgccagaccc cagagccagt ctcagagccc | 5700 |
| tagctccccc gagaaagagt ctcctgagaa agaggaccag gaagaggaaa accagggcgg | 5760 |
| caagggccct ctgctgagca tcctgaaggc cttcaatggc ggcggaggca ggcagcagtg | 5820 |
| ggaactgcag ggcgacagaa gatgccagtc ccagctggaa cgggccaacc tgaggccttg | 5880 |
| cgagcagcac ctgatgcaga aaatccagcg cgacgaggac agctacggcc gggatcctta | 5940 |
| cagccccagc caggaccctt actcccctag ccaggatccc gacagaaggg acccctacag | 6000 |
| ccctagcccc tacgatagaa gaggcgccgg aagcagccag caccaggaaa gatgctgcaa | 6060 |
| cgagctgaac gagtttgaga acaaccagcg ctgcatgtgc gaggccctgc agcagatcat | 6120 |
| ggaaaatcag agcgaccggc tgcagggacg gcagcaggaa cagcagttca agagagagct | 6180 |
| gcggaacctg ccccagcagt gtggactgag agccccccag agatgcgacc tggaagtgga | 6240 |
| aagcggcggc agagatcggt acggcggagg gggcgtgacc ttcagacagg gcggagaaga | 6300 |
| gaatgagtgc cagtttcagc ggctgaacgc ccagaggccc gacaacagaa tcgagagcga | 6360 |
| gggcggctac atcgagacat ggaaccccaa caaccaggaa tttcagtgcg ctggggtggc | 6420 |
| cctgagcagg accgtgctga gaagaaatgc cctgaggcgg cccttctaca gcaacgcccc | 6480 |
| cctggaaatc tacgtgcagc agggcagcgg ctacttcggc ctgatctttc ccggatgccc | 6540 |
| ctccacctat gaggaacccg ctcaggaagg cagacggtat cagagccaga agcctagcag | 6600 |
| acggttccaa gtgggccagg acgatcccag ccaacagcag caggactctc accagaaggt | 6660 |
| gcaccgcttc gacgagggcg acctgatcgc tgtgccaacc ggcgtggcct tctggatgta | 6720 |
| caacgacgag gataccgacg tcgtgaccgt gaccctgagc gacaccagct ccatccacaa | 6780 |
| ccagctggac cagttcccca ggcggtttta cctggccggc aatcaggaac aggaatttct | 6840 |
| gagataccag cagcagcagg gctccagacc ccactacaga cagatcagcc ctagagtgcg | 6900 |
| gggcgacgaa caggaaaatg agggcagcaa catcttctcc ggctttgccc aggaatttct | 6960 |
| gcagcacgcc ttccaggtgg accggcagac cgtggaaaac ctgagaggcg agaacgagag | 7020 |
| agaggaacag ggcgccatcg tgactgtgaa gggcggcctg aggatcctga gccccgacga | 7080 |
| agaggatgag tcctctagaa gcccccccaa ccgccgggaa gagttcgatg aggaccgcag | 7140 |
| cagacctcag cagcggggga agtacgacga gaacaggcgg ggctacaaga acggcatcga | 7200 |
| ggaaacaatc tgcagcgcca gcgtgaagaa gaatctgggc cggtccagca accccgacat | 7260 |
| ctacaatcca caggccggca gcctgcggag cgtgaacgaa ctggatctgc ccatcctggg | 7320 |
| atggctgggc ctgtctgccc agcacggcac catctaccgg aacgccatgt tcgtgcctca | 7380 |
| ctacaccctg aatgcccaca ccatcgtggt ggctctgaac ggccgcgccc acgtccaagt | 7440 |
| ggtggacagc aacggcaatc gggtgtacga tgaagaactg caggaaggac acgtcctggt | 7500 |
| ggtgccccag aattttgccg tggccgccaa ggcccagtcc gagaactatg agtatctggc | 7560 |
| cttcaagacc gacagccggc cctctatcgc caatcaagcc ggcgagaaca gcatcatcga | 7620 |
| caacctgccc gaggaagtgg tggccaacag ctaccggctg cctagagagc aggcccggca | 7680 |
| gctgaagaac aacaaccctt tcaagttctt cgtgccccca ttcgaccacc agagcatgag | 7740 |
| agaggtggcc gaattcacgc tgatccccat cgctgtgggt ggtgccctgg cggggctggt | 7800 |
| cctcatcgtc ctcatcgcct acctcgtcgg caggaagagg agtcacgcag gctaccagac | 7860 |
| tatctagtaa ggatcttttt ccctctgcca aaaattatgg ggacatcatg aagccccttg | 7920 |
| agcatctgac ttctggctaa taaaggaaat ttattttcat tgcaatagtg tgttggaatt | 7980 |
| ttttgtgtct ctcactcgga aggacataag ggcggccgct agc | 8023 |

2. The amino acid sequence of the coding region for the Ara H1/H2/H3 polyprotein chimeric construct, as follows:

```
AraH-LAMP
SEQ ID NO: 9
<220>
<221> SIGNAL
<222> (1)..(27)
<220>
<221> N-LAMP
<222> (28)..(380)
<220>
<221> AraH1
<222> (383)..(983)
<220>
<221> AraH2
<222> (988)..(1138)
<220>
<221> AraH3
<222> (1143)..(1634)
<220>
<221> TM/CYTO
<222> (1637)..(1672)
<400> 7
Met Ala Pro Arg Ser Ala Arg Arg Pro Leu Leu Leu Leu Leu Leu Leu
1 5 10 15
Leu Leu Leu Gly Leu Met His Cys Ala Ser Ala Ala Met Phe Met Val
                        20 25 30
Lys Asn Gly Asn Gly Thr Ala Cys Ile Met Ala Asn Phe Ser Ala Ala
                35 40 45
Phe Ser Val Asn Tyr Asp Thr Lys Ser Gly Pro Lys Asn Met Thr Leu
        50 55 60
Asp Leu Pro Ser Asp Ala Thr Val Val Leu Asn Arg Ser Ser Cys Gly
65 70 75 80
Lys Glu Asn Thr Ser Asp Pro Ser Leu Val Ile Ala Phe Gly Arg Gly
                                85 90 95
His Thr Leu Thr Leu Asn Phe Thr Arg Asn Ala Thr Arg Tyr Ser Val
                        100 105 110
Gln Leu Met Ser Phe Val Tyr Asn Leu Ser Asp Thr His Leu Phe Pro
                115 120 125
Asn Ala Ser Ser Lys Glu Ile Lys Thr Val Glu Ser Ile Thr Asp Ile
        130 135 140
Arg Ala Asp Ile Asp Lys Lys Tyr Arg Cys Val Ser Gly Thr Gln Val
145 150 155 160
His Met Asn Asn Val Thr Val Thr Leu His Asp Ala Thr Ile Gln Ala
                                165 170 175
Tyr Leu Ser Asn Ser Ser Phe Ser Arg Gly Glu Thr Arg Cys Glu Gln
                        180 185 190
Asp Arg Pro Ser Pro Thr Thr Ala Pro Pro Ala Pro Pro Ser Pro Ser
                195 200 205
Pro Ser Pro Val Pro Lys Ser Pro Ser Val Asp Lys Tyr Asn Val Ser
        210 215 220
Gly Thr Asn Gly Thr Cys Leu Leu Ala Ser Met Gly Leu Gln Leu Asn
225 230 235 240
Leu Thr Tyr Glu Arg Lys Asp Asn Thr Thr Val Thr Arg Leu Leu Asn
                                245 250 255
Ile Asn Pro Asn Lys Thr Ser Ala Ser Gly Ser Cys Gly Ala His Leu
                        260 265 270
Val Thr Leu Glu Leu His Ser Glu Gly Thr Thr Val Leu Leu Phe Gln
                275 280 285
Phe Gly Met Asn Ala Ser Ser Ser Arg Phe Phe Leu Gln Gly Ile Gln
        290 295 300
Leu Asn Thr Ile Leu Pro Asp Ala Arg Asp Pro Ala Phe Lys Ala Ala
305 310 315 320
Asn Gly Ser Leu Arg Ala Leu Gln Ala Thr Val Gly Asn Ser Tyr Lys
                                325 330 335
Cys Asn Ala Glu Glu His Val Arg Val Thr Lys Ala Phe Ser Val Asn
                        340 345 350
Ile Phe Lys Val Trp Val Gln Ala Phe Lys Val Glu Gly Gly Gln Phe
                355 360 365
Gly Ser Val Glu Glu Cys Leu Leu Asp Glu Asn Ser Leu Glu Lys Ser
        370 375 380
Ser Pro Tyr Gln Lys Lys Thr Glu Asn Pro Cys Ala Gln Arg Cys Leu
385 390 395 400
Gln Ser Cys Gln Gln Glu Pro Asp Asp Leu Lys Gln Lys Ala Cys Glu
                                405 410 415
Ser Arg Cys Thr Lys Leu Glu Tyr Asp Pro Arg Cys Val Tyr Asp Pro
                        420 425 430
Arg Gly His Thr Gly Thr Thr Asn Gln Arg Ser Pro Pro Gly Glu Arg
                435 440 445
Thr Arg Gly Arg Gln Pro Gly Asp Tyr Asp Asp Asp Arg Arg Gln Pro
        450 455 460
Arg Arg Glu Glu Gly Gly Arg Trp Gly Pro Ala Gly Pro Arg Glu Arg
465 470 475 480
Glu Arg Glu Glu Asp Trp Arg Gln Pro Arg Glu Asp Trp Arg Arg Pro
                                485 490 495
Ser His Gln Gln Pro Arg Lys Ile Arg Pro Glu Gly Arg Glu Gly Glu
                        500 505 510
Gln Glu Trp Gly Thr Pro Gly Ser His Val Arg Glu Glu Thr Ser Arg
                515 520 525
Asn Asn Pro Phe Tyr Phe Pro Ser Arg Arg Phe Ser Thr Arg Tyr Gly
        530 535 540
Asn Gln Asn Gly Arg Ile Arg Val Leu Gln Arg Phe Asp Gln Arg Ser
545 550 555 560
Arg Gln Phe Gln Asn Leu Gln Asn His Arg Ile Val Gln Ile Glu Ala
                                565 570 575
Lys Pro Asn Thr Leu Val Leu Pro Lys His Ala Asp Ala Asp Asn Ile
                        580 585 590
Leu Val Ile Gln Gln Gly Gln Ala Thr Val Thr Val Ala Asn Gly Asn
                595 600 605
Asn Arg Lys Ser Phe Asn Leu Asp Glu Gly His Ala Leu Arg Ile Pro
        610 615 620
Ser Gly Phe Ile Ser Tyr Ile Leu Asn Arg His Asp Asn Gln Asn Leu
625 630 635 640
Arg Val Ala Lys Ile Ser Met Pro Val Asn Thr Pro Gly Gln Phe Glu
                                645 650 655
Asp Phe Phe Pro Ala Ser Ser Arg Asp Gln Ser Ser Tyr Leu Gln Gly
                        660 665 670
Phe Ser Arg Asn Thr Leu Glu Ala Ala Phe Asn Ala Glu Phe Asn Glu
                675 680 685
Ile Arg Arg Val Leu Leu Glu Glu Asn Ala Gly Gly Glu Gln Glu Glu
        690 695 700
Arg Gly Gln Arg Arg Trp Ser Thr Arg Ser Ser Glu Asn Asn Glu Gly
705 710 715 720
Val Ile Val Lys Val Ser Lys Glu His Val Glu Glu Leu Thr Lys His
                                725 730 735
Ala Lys Ser Val Ser Lys Lys Gly Ser Glu Glu Glu Gly Asp Ile Thr
                        740 745 750
Asn Pro Ile Asn Leu Arg Glu Gly Glu Pro Asp Leu Ser Asn Asn Phe
                755 760 765
Gly Lys Leu Phe Glu Val Lys Pro Asp Lys Lys Asn Pro Gln Leu Gln
        770 775 780
Asp Leu Asp Met Met Leu Thr Cys Val Glu Ile Lys Glu Gly Ala Leu
785 790 795 800
Met Leu Pro His Phe Asn Ser Lys Ala Met Val Ile Val Val Val Asn
                                805 810 815
Lys Gly Thr Gly Asn Leu Glu Leu Val Ala Val Arg Lys Glu Gln Gln
                        820 825 830
Gln Arg Gly Arg Arg Glu Glu Glu Glu Asp Glu Asp Glu Glu Glu Glu
                835 840 845
Gly Ser Asn Arg Glu Val Arg Arg Tyr Thr Ala Arg Leu Lys Glu Gly
        850 855 860
Asp Val Phe Ile Met Pro Ala Ala His Pro Val Ala Ile Asn Ala Ser
865 870 875 880
Ser Glu Leu His Leu Leu Gly Phe Gly Ile Asn Ala Gln Asn Asn His
                                885 890 895
Arg Ile Phe Leu Ala Gly Asp Lys Asp Asn Val Ile Asp Gln Ile Glu
                        900 905 910
Lys Gln Ala Lys Asp Leu Ala Phe Pro Gly Ser Gly Glu Gln Val Glu
                915 920 925
Lys Leu Ile Lys Asn Gln Lys Glu Ser His Phe Val Ser Ala Arg Pro
        930 935 940
Gln Ser Gln Ser Gln Ser Pro Ser Ser Pro Glu Lys Glu Ser Pro Glu
945 950 955 960
Lys Glu Asp Gln Glu Glu Glu Asn Gln Gly Gly Lys Gly Pro Leu Leu
                                965 970 975
Ser Ile Leu Lys Ala Phe Asn Gly Gly Gly Gly Arg Gln Gln Trp Glu
                        980 985 990
Leu Gln Gly Asp Arg Arg Cys Gln Ser Gln Leu Glu Arg Ala Asn Leu
                995 1000 1005
Arg Pro Cys Glu Gln His Leu Met Gln Lys Ile Gln Arg Asp Glu
        1010 1015 1020
Asp Ser Tyr Gly Arg Asp Pro Tyr Ser Pro Ser Gln Asp Pro Tyr
        1025 1030 1035
Ser Pro Ser Gln Asp Pro Asp Arg Arg Asp Pro Tyr Ser Pro Ser
        1040 1045 1050
Pro Tyr Asp Arg Arg Gly Ala Gly Ser Ser Gln His Gln Glu Arg
        1055 1060 1065
Cys Cys Asn Glu Leu Asn Glu Phe Glu Asn Asn Gln Arg Cys Met
        1070 1075 1080
Cys Glu Ala Leu Gln Gln Ile Met Glu Asn Gln Ser Asp Arg Leu
        1085 1090 1095
Gln Gly Arg Gln Gln Glu Gln Gln Phe Lys Arg Glu Leu Arg Asn
        1100 1105 1110
Leu Pro Gln Gln Cys Gly Leu Arg Ala Pro Gln Arg Cys Asp Leu
        1115 1120 1125
Glu Val Glu Ser Gly Gly Arg Asp Arg Tyr Gly Gly Gly Gly Val
        1130 1135 1140
Thr Phe Arg Gln Gly Gly Glu Glu Asn Glu Cys Gln Phe Gln Arg
        1145 1150 1155
Leu Asn Ala Gln Arg Pro Asp Asn Arg Ile Glu Ser Glu Gly Gly
        1160 1165 1170
Tyr Ile Glu Thr Tip Asn Pro Asn Asn Gln Glu Phe Gln Cys Ala
        1175 1180 1185
Gly Val Ala Leu Ser Arg Thr Val Leu Arg Arg Asn Ala Leu Arg
        1190 1195 1200
Arg Pro Phe Tyr Ser Asn Ala Pro Leu Glu Ile Tyr Val Gln Gln
        1205 1210 1215
Gly Ser Gly Tyr Phe Gly Leu Ile Phe Pro Gly Cys Pro Ser Thr
        1220 1225 1230
Tyr Glu Glu Pro Ala Gln Glu Gly Arg Arg Tyr Gln Ser Gln Lys
        1235 1240 1245
Pro Ser Arg Arg Phe Gln Val Gly Gln Asp Asp Pro Ser Gln Gln
        1250 1255 1260
Gln Gln Asp Ser His Gln Lys Val His Arg Phe Asp Glu Gly Asp
        1265 1270 1275
Leu Ile Ala Val Pro Thr Gly Val Ala Phe Trp Met Tyr Asn Asp
        1280 1285 1290
Glu Asp Thr Asp Val Val Thr Val Thr Leu Ser Asp Thr Ser Ser
        1295 1300 1305
Ile His Asn Gln Leu Asp Gln Phe Pro Arg Arg Phe Tyr Leu Ala
        1310 1315 1320
Gly Asn Gln Glu Gln Glu Phe Leu Arg Tyr Gln Gln Gln Gln Gly
        1325 1330 1335
Ser Arg Pro His Tyr Arg Gln Ile Ser Pro Arg Val Arg Gly Asp
        1340 1345 1350
Glu Gln Glu Asn Glu Gly Ser Asn Ile Phe Ser Gly Phe Ala Gln
        1355 1360 1365
Glu Phe Leu Gln His Ala Phe Gln Val Asp Arg Gln Thr Val Glu
        1370 1375 1380
Asn Leu Arg Gly Glu Asn Glu Arg Glu Glu Gln Gly Ala Ile Val
        1385 1390 1395
Thr Val Lys Gly Gly Leu Arg Ile Leu Ser Pro Asp Glu Glu Asp
        1400 1405 1410
Glu Ser Ser Arg Ser Pro Pro Asn Arg Arg Glu Glu Phe Asp Glu
        1415 1420 1425
Asp Arg Ser Arg Pro Gln Gln Arg Gly Lys Tyr Asp Glu Asn Arg
        1430 1435 1440
Arg Gly Tyr Lys Asn Gly Ile Glu Glu Thr Ile Cys Ser Ala Ser
        1445 1450 1455
Val Lys Lys Asn Leu Gly Arg Ser Ser Asn Pro Asp Ile Tyr Asn
        1460 1465 1470
Pro Gln Ala Gly Ser Leu Arg Ser Val Asn Glu Leu Asp Leu Pro
        1475 1480 1485
Ile Leu Gly Trp Leu Gly Leu Ser Ala Gln His Gly Thr Ile Tyr
        1490 1495 1500
Arg Asn Ala Met Phe Val Pro His Tyr Thr Leu Asn Ala His Thr
        1505 1510 1515
Ile Val Val Ala Leu Asn Gly Arg Ala His Val Gln Val Val Asp
        1520 1525 1530
Ser Asn Gly Asn Arg Val Tyr Asp Glu Glu Leu Gln Glu Gly His
        1535 1540 1545
Val Leu Val Val Pro Gln Asn Phe Ala Val Ala Ala Lys Ala Gln
        1550 1555 1560
Ser Glu Asn Tyr Glu Tyr Leu Ala Phe Lys Thr Asp Ser Arg Pro
        1565 1570 1575
Ser Ile Ala Asn Gln Ala Gly Glu Asn Ser Ile Ile Asp Asn Leu
        1580 1585 1590
Pro Glu Glu Val Val Ala Asn Ser Tyr Arg Leu Pro Arg Glu Gln
        1595 1600 1605
Ala Arg Gln Leu Lys Asn Asn Asn Pro Phe Lys Phe Phe Val Pro
        1610 1615 1620
Pro Phe Asp His Gln Ser Met Arg Glu Val Ala Glu Phe Thr Leu
        1625 1630 1635
Ile Pro Ile Ala Val Gly Gly Ala Leu Ala Gly Leu Val Leu Ile
        1640 1645 1650
Val Leu Ile Ala Tyr Leu Val Gly Arg Lys Arg Ser His Ala Gly
        1655 1660 1665
Tyr Gln Thr Ile
        1670
```

1. An isolated or purified nucleic acid comprising, in sequential order:
a sequence encoding a signal sequence;
a sequence encoding an intra-organelle stabilizing domain;
a sequence encoding an allergen domain, wherein the allergen domain comprises at least one allergen that does not include a naturally-occurring signal sequence for the allergen;
a sequence encoding a transmembrane domain; and
a sequence encoding an endosomal/lysosomal targeting domain.

2. The nucleic acid of claim 1, wherein the intra-organelle stabilizing domain comprises a sequence encoding a lysosomal associated membrane protein (LAMP).

3. The nucleic acid of claim 1, wherein the intra-organelle stabilizing domain comprises a sequence encoding the LAMP polypeptide, DC-LAMP, LAMP2, LAMP-3, LIMP II, or ENDOLYN.

4. The nucleic acid of claim 1, wherein the sequence encoding an allergen domain comprises a sequence that encodes two or more allergenic epitopes.

5. The nucleic acid of claim 1, wherein the sequence encoding an allergen domain comprises a sequence that encodes two or more allergens.

6. The nucleic acid of claim 5, wherein the allergens are from two or more different species.

7. The nucleic acid of claim 1, wherein the allergen is the Cry J1, Cry J2, Cry J3, CJP-4, CJP-6, CJP-8, CPA63, Cha o 1, Jun a 1, Jun v 1, Cup a 1, Jun o 1, Cup s 1, Cha o 2, Jun a 2, Cup a 2, Jun a 3, Jun r 3, Cup s 3, Cup a 3, Ch4A, Ch4-1, PT-1, or LTP allergen, or a portion thereof having at least one allergenic epitope.

8. The nucleic acid of claim 1, which is a DNA vaccine that induces an immune response in a host.

9. The nucleic acid of claim 1, wherein the allergen domain comprises a sequence that encodes two or more allergens, each from the same species.

10. The nucleic acid of claim 1, wherein the allergen domain comprises a sequence that encodes two or more allergens, each from a different species.

11. A method of reducing, eliminating, or preventing an allergic reaction in a subject, said method comprising administering to the subject the DNA vaccine of claim 1 in an amount sufficient to reduce or eliminate production of IgE response specific for the allergenic epitope.

12. The method of claim 11, wherein the method prevents the allergic reaction.

13. The method of claim 12, wherein the method reduces, eliminates, or prevents at least one clinical allergy symptom.

14. The method of claim 13, wherein the method prevents at least one clinical allergy symptom.
